SEQUENCE LISTING (17 sequences)

SEQ ID NO: 1 - protein, 4 aa, Artificial Sequence
  Collagen-derived peptide
DGEA

SEQ ID NO: 2 - protein, 6 aa, Artificial Sequence
  Collagen-derived peptide
GFPGER

SEQ ID NO: 3 - protein, 13 aa, Artificial Sequence
  cell surface binding factor of the disclosure
IPVSLRSGAGPEG

SEQ ID NO: 4 - protein, 12 aa, Artificial Sequence
  cell surface binding factor of the disclosure
GPLGLAGGERDG

SEQ ID NO: 5 - protein, 4 aa, Artificial Sequence
  cell surface binding factor of the disclosure
GFLG

SEQ ID NO: 6 - protein, 8 aa, Artificial Sequence
  cell surface binding factor of the disclosure
GPMGIAGQ

SEQ ID NO: 7 - protein, 22 aa, Artificial Sequence
  cell surface binding factor domain of the disclosure including cleavable
GGGGIPVSLRSGAGPEGDGEAY

SEQ ID NO: 8 - protein, 8 aa, Artificial Sequence
  miRNA target sequence that directs exosomal sorting
GGGGRGDY

SEQ ID NO: 9 - protein, 4 aa, Artificial Sequence
  peptide used to provide cell surface binding factor of the disclosure
GGAG

SEQ ID NO: 10 - protein, 9 aa, Artificial Sequence
  peptide used to provide cell surface binding factor of the disclosure
GGGGDGEAY

SEQ ID NO: 11 - protein, 6 aa, Artificial Sequence
  peptide used to provide cell surface binding factor of the disclosure
GFPGER

SEQ ID NO: 12 - DNA, 25 bp, Artificial Sequence
  has-miR15a-5p primer
gggtagcagcacataatggtttgtg

SEQ ID NO: 13 - DNA, 25 bp, Artificial Sequence
  has-miR15b-5p primer
gggtagcagcacatcatggtttaca

SEQ ID NO: 14 - DNA, 24 bp, Artificial Sequence
  has-miR16-5p primer
ggtagcagcacgtaaatattggcg

SEQ ID NO: 15 - DNA, 24 bp, Artificial Sequence
  has-miR424-5p primer
ggcagcagcaattcatgttttgaa

SEQ ID NO: 16 - DNA, 21 bp, Artificial Sequence
  has-miR497-5p primer
cagcagcacactgtggtttgt

SEQ ID NO: 17 - DNA, 18 bp, Artificial Sequence
  has-miR3960 primer
gcggaggcgggggaaaaa

(19) United States
(12) Patent Application Publication
(10) Pub. No.: US 2021/0283186 A1
(43) Pub. Date: Sep. 16, 2021

(54) Engineered Exosomes for Medical Applications

(71) Applicant: THE BOARD OF TRUSTEES OF THE UNIVERSITY OF ILLINOIS, Urbana, IL (US)

(72) Inventors: Sriram RAVINDRAN, Chicago, IL (US); Praveen GAJENDRAREDDY, Elk Grove Village, IL (US); Lyndon COOPER, Chicago, IL (US); Chun-Chieh HUANG, Chicago, IL (US)

(21) Appl. No.: 17/261,168

(22) PCT Filed: Jul. 16, 2019
(86) PCT No.: PCT/US2019/042096

§ 371 Date: Jan. 18, 2021

Related U.S. Application Data
(60) Provisional application No. 62/698,650, filed on Jul. 16, 2018.

(51) Int. Cl. A61K 35/28; C12N 15/113; C12N 5/0775; A61K 9/19; A61K 9/00; A61P 9/10; A61P 27/02; A61K 9/06; A61K 47/42; A61L 27/36; A61L 27/56

(52) U.S. Cl. CPC A61K 35/28; A61L 2430/20; C12N 5/0663; C12N 5/0664; A61K 9/19; A61K 9/0019; A61P 9/10; A61P 27/02; A61K 9/06; A61K 47/42; A61L 27/3604; A61L 27/56; C12N 2310/141; C12N 2501/999; C12N 2501/25; C12N 2500/02; C12N 15/113

(57) ABSTRACT

This invention relates to exosome compositions and methods of using them.

### CROSS REFERENCE TO RELATED APPLICATIONS

This application claims the benefit of U.S. Provisional Application No. 62/698,650, filed Jul. 16, 2018, which is incorporated herein by reference for all purposes.

### BACKGROUND OF DISCLOSURE

### Field of Invention

This invention relates to compositions and methods for making and using exosomes to treat various disorders.

### Technical Background

Exosomes are cell-derived nano scale (40-150 nm), lipid layered spheroids packed with unique cell-type specific protein and/or nucleic acids. Parental cells secrete exosomes to transfer this “information” to effector cells. This results in a signaling process that can provide parental cell influence on target cell function. Current studies of exosome function(s) highlight their important roles in modulating cellular signaling in immunology, cancer biology and regenerative medicine.

Exosomes derived from some types of cells, such as mesenchymal stem cells and dendritic cells have therapeutic potential and can be considered efficient agents against various disorders. However, many challenges for the development of exosome-based therapeutics are known in the art. Specifically, heterogeneity and low productivity of art-recognized methods for producing exosome formulations is the major barrier for their therapeutic application. Development and optimization of producing methods, including methods for isolating and storing exosome formulations, are required for accomplishing exosome-based therapeutics. Moreover, improvement of delivery efficiency of exosomes is important for their therapeutic application, which can include treatment of bone damage and treatment of neurological disorde

Osteoimmunology is a central phenomenon controlling adult bone health, disease and regeneration. Failure of osteogenesis (i.e., the formation of bones) complicates dentoalveolar and orthopedic therapies. The biologic and therapeutic control of bone repair is linked to responses of injury that involve activation of the immune system. Facture repair involves responses mediated by inflammatory cytokines. Therefore, there exists a need in this art for new methods to treat bone diseases that will promote bone repair yet minimize activation of the immune system.

Neurological disorders are complex in both origin and progression. Several factors contribute to injury or damage of nerve cells. These factors include physical traumas such as head traumas, sport accidents and vehicle accidents; chemical traumas such as drug or alcohol abuse and exposure to environmental chemicals; metabolic traumas such as epileptic seizure, spinal cord ischemia, and cerebral ischemia; and complicated trauma (or complex migraine) that are associated with high prevalence of stroke or transient ischemic attack during migraine attacks.

Central nervous system ischemia triggers both restorative and degenerative processes. Restorative processes are neurotrophic in nature, regenerative and reparative. These drive cells and tissues toward health and normal function. Degenerative processes lead to loss of function, cell death, and can spread from the area directly affected by the primary insult to more diffuse areas of the central nervous system. Following ischemic trauma such as stroke to the central nervous system, degenerative processes tend to predominate, leading to progressive secondary damage or injury and its sequelae of adverse health conditions or disability. It has also been suggested that normally restorative processes can be altered in certain ways to become degenerative. Secondary injury is caused or brought about by cascades of cellular and metabolic processes. These secondary injury processes are spread over a space and time continuum. For instance, after spinal cord injury changes can be observed in neuronal function even in remote areas of the central nervous system including the brain, and these processes follow time courses of hours, days, weeks and even months.

Neurological disorders have proved to be some of the most difficult types of disease to treat. In fact, for some neurological diseases, there are no drugs available that provide significant therapeutic benefit. The difficulty in providing therapy is all the more tragic given the devastating effects these diseases have on their victims. Therefore, there is a need for new and effective methods to treat disorders or damage of the neurological systems.

### SUMMARY OF THE DISCLOSURE

This disclosure provides exosome compositions and methods of using them.

As described below, in one aspect, the disclosure provides a composition comprising isolated engineered exosomes from mesenchymal stem cells (MSCs), each exosome comprising at least one factor that is: an osteoinductive factor, a neuronal regeneration factor, an immunomodulatory factor, an extracellular matrix binding factor, or a combination thereof, wherein the at least one factor is present at a higher amount in the engineered exosome than the amount present in a naturally occurring cell-derived exosome.

In another aspect, the disclosure provides a method of preparing a composition of the disclosure, comprising engineering stem cells to contain at least one factor that is: an osteoinductive factor, a neuronal regeneration factor, an immunomodulatory factor, and an extracellular matrix binding factor at a higher amount than stem cells that are not engineered; and isolating the exosome from the cells.

Another aspect of the disclosure is a method for treating an eye disorder in an individual comprising delivering a composition of isolated exosomes to vitreous humour of the individual, wherein the exosomes are enriched in regenerative factors endogenous to stem cells.

### BRIEF DESCRIPTION OF THE DRAWINGS

The accompanying drawings are included to provide a further understanding of the methods and compositions of the disclosure, and are incorporated in and constitute a part of this specification. The drawings illustrate one or more embodiment(s) of the disclosure and, together with the description, serve to explain the principles and operation of the disclosure.

FIG. 1. Generation and testing of biomimetic FATE as nano-modulators of Stem Cell Lineage Determination (SCLD) for tissue-engineering applications.

FIG. 2. Exosome analysis. (A) Representative transmission electron microscopy (TEM) image of exosomes. (B, C) Representative TEM images of exosomes that were immunostained for CD63 using 10 nm gold particles. The electron dense black dots represent positive staining. (D) Immunoblots showing the presence of exosome markers CD63 and CD9 in the exosome protein lysates.

FIG. 3. Workflow schematic for generation of α5 FATE.

FIG. 4. Exosome binding to COL1. Dose dependent and saturable binding of exosomes to collagen type 1 (COL1) coated (5 μg) assay plates. 1 μl of exosome suspension corresponds to exosomes from 10,000 cells.

FIG. 5. Exosome binding to fibronection (FN). (A) Confocal microscopic image of exosomes (immunostained for marker CD63) bound to cell-secreted FN in the extracellular matrix (ECM) of decellularized human mesenchymal stem cells (HMSCs). The arrows in the merged image shows areas of colocalization of exosomes with FN. (B) Confocal microscopic image showing that blocking exosome integrins with 2 mM RGD peptide blocks exosome binding to FN.

FIG. 6. Representative TEM images of exosomes from HMSCs (left panel) and HMSCs constitutively expressing integrin α5 (right panel) immunogold labeled for integrin α5. The arrows point to increased presence of the integrin α5 on the exosome membranes indicating that increasing integrin expression on the parent cell plasma membrane increases its expression on the exosome membrane.

FIG. 7. Saturable endocytosis of fluorescently labeled exosome by MSCs. Relative fluorescence in arbitrary units is shown. Endocytosis levels off with increased exosome delivery as the cells mechanisms become saturated.

FIG. 8. 3D reconstruction of z-stack confocal images showing endocytosis collagen bound exosome (represented by Osteo Exo) by HMSCs (represented by Actin).

FIG. 9. Table depicting increased potency of osteogenic exosomes to induce HMSC differentiation.

FIG. 10. Workflow for production of osteoinductive exosomes.

FIG. 11. Endocyctosis of exosome is not integrin mediated. (A) Confocal images showing fluorescently labeled exosomes (Exo) endocytosed by HMSCs (tubulin). (B) Confocal images showing endocytosis of fluorescently labeled exosomes (Exo) pretreatment with 2.5 mM RGD peptide to block integrins. Note that endocytosis of exosomes was not blocked after integrin blocking (B). DAPI, 4′,6-diamidino-2-phenylindole.

FIG. 12. Representative micrographs of sections from control (A1, B1, C1, D1, E1) and osteogenic exosome-containing (A2, B2, C2, D2, E2) groups of collagen sponges seeded with HMSCs. Scaffolds were implanted for 4 weeks subcutaneously in nude mice and immunostained for phosphorylated proteins (p-STT), DMP1, VEGF and BMP2. Note the increased expression of these proteins in A2, B2, C2 and D2. (E1 and E2) are H&E stained sections. The arrows in E2 point to RBC containing capillaries showing vascularization in the group containing exosomes. (F) A graphical representation of serial von Kossa and alizarin red stained sections that shows exosome mediated increase in mineralization in the form of calcium phosphate. Error bars represent mean+/−SD and * represents statistical significance with respect to control (student's t-test P<0.01).

FIG. 13. Increased expression of Let7a and miR218 in osteogenic MSC exosomes compared to control MSC exosomes.

FIG. 14. Dose dependent reduction in endocytosis of fluorescently labeled MSC exosomes in the presence of heparin. (*) Represents statistical significance with respect to control (#) represents significance between indicated groups (Students t-test (p<0.05)).

FIG. 15. Workflow and experimental groups for in vivo experiments.

FIG. 16. Model of MSC immunomodulation during osteogenesis involves altering macrophage (MØ) M1/M2 polarization. Reducing the ratio of proinflammatory M1 MØ to anti-inflammatory M2MØ exosomes promotes osteoinduction and regeneration.

FIG. 17. Venn diagram showing results of miRNAseq analysis of MØ polarized exosomes reveal a small set of polarization-specific miRNAs. MØ were polarized using LPS+IFNγ to M1 and IL4 to M2 phenotypes; Exosomes were isolated and RNA was prepared. Small RNA libraries were constructed and subjected to sequencing (Illumine). Sequences aligned to the mouse genome were mapped to mmiRBase_v.19 and normalized to reads per million. The highly expressed miRNAs were compared manually as shown.

FIG. 18. Table showing polarized MØ exosome miRNAs and their known relationship to osteoinduction/osteogenesis. There are few miRNA uniquely expressed in the polarized MØ. In M2 MØ, two of the three miRNAs are implicated in the positive regulation of osteoinduction. An M2-enriched MØ population can enhance bone repair.

FIG. 19. Graph comparing IL-1beta. IL-6 and IL-10 in cells. MØ were treated with MSCcont and MSCTNFα exosome for 24 hours. Total RNA was isolated and cytokine expression was measured by quantitative reverse transcription-polymerase chain reaction (qPCR) (n=4;*p, 0.05, **=p, 0.01).

FIG. 20. Assays for M1 polarization pathway members (top); Assays for M2 polarization pathway members (bottom). Known experimental methods for inhibiting the molecules and a means of result readout are shown.

FIG. 21. Table of M1 Inhibitors and their induction in response to TNFα. Exosomes from MSCs treated with PBS or 10 ng/ml TNFα for 18 hours were prepared and small RNAs were isolated. The levels of 5 known miRNA inhibitors of M1 signaling pathways were quantified by qRT-PCR. All were induced by TNFα treatment of MSCs.

FIGS. 22 and 23. MSC Exosomes alter the ratio of M1/M2 exosomes during bone regeneration. FIG. 22, left: Collagen scaffolds containing 3×10⁶ MSC exosomes were placed in calvaria (skullcap) defects in rats to assay bone regeneration. FIG. 22, right: At 3 weeks, immunostaining for M1 (α-Arg1) and M2 (α-CD206) was performed. Staining revealed reduced M1 MØ in treated defects. FIG. 23: The MSC exosome-mediated reduced M1 and increased M2 population (↓M1/M2) suggests that MSC exosomes promote a regenerative MØ population for healing.

FIG. 24. Schematic for MØ polarization signaling pathways. The relevant exosome population is shown on top.

FIG. 25. Characterization of Exosomes. Particle tracking analysis of isolated extracellular vesicles (Evs) showed a size distribution that fit the exosome profile for both MSC and MØ. Immunoblotting (labeled Western blot) showed the presence of exosome markers CD63 and CD9 for both cells. TEM of immunogold labeled vesicles showed the presence of vesicles labeled positively for CD63 (10 nm gold labeled) falling within the prescribed size distribution of exosomes.

FIG. 26. Endocytosis of MSC exosomes by MØ. A) Dose dependent endocytosis of fluorescently labeled MSC exosomes by MØ. B) A confocal image of MØ (tubulin) with MSC exosomes within MØ (tubulin). Nuclei are indicated (central dark areas, DAPI stained).

FIG. 27. Primary mouse MØ polarization. Mouse bone marrow MØ were treated with LPS/IFNγ (M1) or IL-4 (M2) for 24 hours and fixed for immunostaining or lysed for qPCR analysis of polarization markers. Top: M1 express high levels of iNOS, IL 1β, TNFα; M2 express Arg1, CD206 and FIZZ1. Bottom: immunostaining affirms M1 specific iNOS and M2 elevated CD206 expression.

FIG. 28. Table showing phenotypic markers of MØ polarization.

FIG. 29. Polarity-specific effects of MØ exosomes on MSCs. Left: Bar graph showing expression of BMP2 and 9 in MSCs 72 hours following treatment with M0, M1, or M2 exosomes. Fold change determined by qPCR (n=4). b) Bar graph representing transactivation of the BMP2-responsive SBE12 plasmid following MSC treatment with M0, M1 or M2 exosomes+/−50 ng/ml rhBMP2. Note the potentiated BMP2 signaling with M0 or M2 exosome treatment (*=p<0.01; **=p<0.001).

FIG. 30. MicroCT and immunohistochemical evaluation of MØ exosome-mediated mouse calvaria bone regeneration. 3.5 mm mouse calvaria defects were treated with 3.5 mm diameter collagen scaffolds containing either PBS, M1 or M2 MØ exosomes (4.0×10⁸ exosomes/calvaria). Top left: Representative reconstructed μCT images of 3 and 6 week treated defects reveal positive effects of M2 exosome treatment. Top right: Quantifying mineralized tissue by μCT revealed marked bone regeneration at 6 weeks only in the M2 exosome-treated calvaria (calculated in Matlab and statistically compared (n=6; *=p<0.05)). Bottom: Confocal microscopy of BMP2 and BSP expression in healing calvaria 6 weeks after placement of collagen scaffolds containing either PBS, M1 or M2 MØ exosomes. M1 exosomes impared osteogenesis (and BMP2 and BSP expression). M2 exosome treatment supported osteogenesis/bone regeneration (and BMP/BSP gene expression) at 6 weeks.

FIGS. 31 and 32. Increased expression of miRNA in exosomes effectively targets cell functions. FIG. 31, left: Schematic showing process. miR424 (proliferative function) was cloned into XMIR plasmid and resulting lentivirus was transduced into R28 cells. FIG. 31, Right: miR 424 expression was analyzed by QPCR, and miR424 abundance was increased 115-fold (vs. control) in exosomes. Exosomes were characterized (CD9, CD63, nanocyte (not shown)). FIG. 32, left: The miR424 exosomes were taken up by cells. Right: The exosomes induced increased proliferation relative to control exosomes.

FIG. 33. Engineered exosomes promote osteogenesis. Exosomes from BMP2-expressing cells that over expressed miRNAs (˜5 to 11 fold) that down regulated the BMP inhibitors BAMBI and SMAD7 were produced. These exosomes ((4.0E⁸)/calvaria) increased osteogenic gene in vitro stimulated bone regeneration in vivo. miR424 is upregulated in BMP2 exosomes.

FIG. 34. Monocyte depletion impairs bone healing. A) The number of F4/80-CD11 b double positive cells in peripheral blood of Control and MaFIA mice treated with AP20187 measured at day 3 by flow cytometry indicates a significant reduction in the monocytes. B) micro CT images of control and AP20185 treated (×2 weeks) mice calvaria with 3 mm defects after 28 days post-surgery. This affirms previous studies in fracture and tibia defect bone repair models in the MaFIA mouse.

FIG. 35. Automated Calculation of Bone Volumes from μCT data. a) Low-resolution 3D rendering of the μCT imaged calvaria. The black circle=experimental region (osteotomy), dashed circle=control region (intact); markers=anterior and posterior ends of the sagittal suture defining the main axis of the cranium. b) High-res 3D rendering showing the relative bone densities, as percentage of the maximum density observed. Top, the bone density of thin coronal sections is shown. Lighter areas are higher density.

FIG. 36. E Analyses of miR 424 exosomes. A. QPCR data showing exosome specific overexpression of miR424 B. Engineered exosomes show the presence of exosome markers C. Endocytosis of control exosomes D. Endocytosis of engineered exosomes showing that altering miRNA content does not affect the endocrine process.

FIG. 37. Endocytosis of HMSC miR424 by R28 cells.

FIG. 38. Endocytosis of dental pulp stem cell (DPSC) miR424 by R28 cells.

FIG. 39. Engineered exosomes rescue ischemic retinal cells. To mimic ischemic conditions, R28 retinal cells were subjected to oxygen and glucose deprivation (OGD). To test the hypothesis if exosomes can rescue R28 cells from OGD-mediated cell death, the R28 cells were subjected to OGD conditions for 6 h and later were treated with exosomes overnight. The cytotoxicity was measured from LDH (LDH is an enzyme that is released when cells are dying) released by the cells. As seen in the figure, OGD conditions caused more than 50% of cell death. Conversely, when same were treated with DPSC exosomes showed significant reduction in % cell death as compared to cells with absence of exosomes. The same experiment was performed using DPSC miR424 derived exosomes. Similar results were obtained. When compared, DPSC miR424 derived exosomes proved more effective than DPSC exosomes. Also, condition media depleted of exosomes were tested and fewer protective effects were seen implying that the protective effects are due to the presence of exosomes (data not shown).

FIG. 40. Proliferation of Retinal Cell Line (R28) cells treated with miR 424 exosomes versus control exosomes. Proliferation is shown relative to untreated R28 cells. A lactate dehydrogenase (LDH) assay was used to assess proliferation.

FIG. 41. Characterization of MSC derived EVs. (A) Nanoparticle Tracking Analysis (NTA) histogram demonstrating MSC-EVs' size distribution after isolation using centrifugation and EV Exo-quick Isolation Reagent. In the insert, mean and mode for particle size are displayed along with concentration. MSC-EVs showed a modal size of 93 nm, peaks at 89 and 141 nm, and the presence of few large vesicles (shown as larger peaks at higher diameters) indicating that the majority of the MSC-EVs are likely exosomes. (B) Western blot illustrating the characteristic surface markers of exosomes, CD63, CD9, CD81, and HSP70α, present in MSC-EV preparations, but not in MSC-conditioned medium (CM) depleted of EVs. Molecular weight markers are on left of each blot. (C) Transmission electron microscopic (TEM) image of cup-shaped MSC-EVs isolated from MSCs with diameters of approximately 100 nm, consistent with exosomal size. (D) Immunogold labeling of MSC-EVs with CD63 antibody to exosome surface markers, again demonstrating that the MSC-EVs are mainly exosomes. Scale bar are on lower left of panels C and D.

FIG. 42. Endocyctosis of MSC-EVs by R28 cells. (A) Representative confocal micrograph demonstrating endocytosis of fluorescently labeled EVs by R28 cells. The cells were counterstained with primary antibody to tubulin (cytoskeleton, red), and with DAPI to stain the nuclei (blue). Clockwise from the top left are: DAPI (blue), MSC-EVs, composite of DAPI, MSC-EVs, and tubulin. The image on the top right of panel A demonstrates punctae of MSC-EVs (light arrows) and denser concentration of MSC-EVs (dark arrows near center of image), and there is co-localization of MSC-EVs and tubulin within the cytoplasm of the cells (arrows in lower right, composite panel of 2A). Scale bars are on the top of each panel. (B) Graph indicates a dose-dependent and saturable endocytosis of fluorescently labeled MSC-EVs. X-axis is volume of MSC-EVs and Y-axis indicates mean normalized fluorescence units. (C) Quantitative fluorescence measurements of MSC-EV endocytosis at 37° C. and 4° C. showing a decrease in endocytosis at lower temperature. Temperature is on X-axis, and Y-axis is mean normalized fluorescence units. The data represented in panel B and panel C are the mean of 6 individual experiments, and error bars indicated SD. * in panel C represents statistical significance with respect to control (normothermia, P<0.01).

FIG. 43. Heparin sulfate proteoglycans (HSPGs), but not integrins, are involved in endocytosis of MSC-EVs by R28 cells. (A) Increasing doses of RGD peptide to block cell surface integrins did not alter endocytosis of fluorescently labeled MSC-EVs. Y-axis is mean normalized fluorescence units±SD; the X-axis is dose of RGD in mM. No statistical significance was observed (n=6 experiments). (B) Dose-dependent reduction of fluorescently labeled MSC-EV endocytosis after heparin pretreatment to block HSPGs. Data on Y-axis is mean normalized fluorescence units±SD; the X-axis is dose of heparin in μg/ml. *=P<0.05 compared to vehicle (heparin=“0”), n=6 experiments. (C) Representative confocal micrograph showing endocytosis of fluorescently labeled MSC-EVs by R28 cells treated with PBS vehicle (control). (D) Representative confocal micrograph showing no reduction in endocytosis of MSC-EVs after pre-incubation with RGD to block integrins (RGD=“0” is PBS vehicle alone). (E) Representative confocal micrograph showing reduction in endocytosis of MSC-EVs after they were pre-incubated with heparin to block HSPGs. (For C, D, and E, from left to right are shown MSC-EVs, DAPI to stain the cell nuclei, anti-tubulin to stain cytoskeleton, and composite of MSC-EVs, DAPI, and tubulin on the far right. Endocytosis can be seen in C and D, in the far right panels, where MSC-EVs are visible inside cells (white arrows), as well as overlapping with tubulin (grey arrows). Scale bars appear on top or bottom of each panel.

FIG. 44. Involvement of the caveolar pathway in MSC-EV endocytosis by R28 cells. (A) Representative confocal images showing endocytosed fluorescently labeled MSC-EVs co-localized with anti-caveolin 1. From left to right are DAPI, MSC-EVs, caveolin-1, and merged. (B) Magnified area of box in A. White arrowheads point to regions of co-localization of caveolin-1 and MSC-EVs. (C) Representative confocal images of endocytosed MSC-EVs counterstained with anti-clathrin. From left to right are DAPI, MSC-EVs, clathrin, and merged. (D) Magnified area of box in C. Note that in contrast to A and B, there is no co-localization of MSC-EVs and clathrin in C and D. (E) Representative confocal images showing endocytosed fluorescently labeled MSC-EVs in R28 cells. From left to right are DAPI, MSC-EVs, anti-tubulin, and merged. MSC-EVs are visible inside the cells in the far right merged panel (shown by grey arrows), or where tubulin and MSC-EVs co-localize (shown by white arrows). (F) Representative confocal images showing endocytosed fluorescently labeled MSC-EVs in R28 cells after pretreatment with methyl-β-cyclodextrin (MBCD) to disrupt R28 cell membrane cholesterol. From left to right are DAPI, MSC-EVs, tubulin, and merged. (G) Quantitation of MBCD effect on endocytosis of MSC-EVs into R28 cells. There was a significant dose dependent reduction in MSC-EV uptake with increasing doses of MBCD. Data on the Y-axis in mean normalized fluorescence units±SD; the X-axis is dose of MBCD in mM. *=P<0.05 compared to control, n=6 experiments.

FIGS. 45 and 46. EVs protect retinal cells from OGD-induced cell death. FIG. 45: Dose dependent effect of MSC-EVs on oxygen glucose deprivation (OGD) induced cytotoxicity of R28 cells as measured by lactate dehydrogenase (LDH) assay. Note the decrease in cell death from OGD with increasing dosage of MSC-EVs with saturation at 10⁵ EV/ml. In A, data is presented as percentage cytotoxicity on Y-axis (% cell death, LDH, mean±SD), and X-axis in concentration of MSC-EVs in particles/ml. n=6 experiments*=P<0.05 vs OGD alone. FIG. 46, top: Representative flow cytometry results for the presence of EdU-positive cells after OGD with and without EVs. The percentages within the graphs in bold indicated the % of proliferating cells. Conditioned medium (CM) without EVs (CM-Exo), and PBS (ctrl) were controls. Exo=Evs. FIG. 46, bottom: Graphical representation of results in (B). Y-axis is % EdU-positive cells (mean±SD). n=4 experiments, *=p<0.05 normoxia vs OGD, #=p<0.05 vs control (“ctrl”, OGD+PBS). Both CM and Exo prevented the loss of proliferation in cells subjected to OGD, while CM-Exo showed no effect. Although there was a small decrease in the proliferation in normoxic cells treated with EVs, there was no significant difference from the control.

FIGS. 47 and 48. MSC-EVs enhance functional recovery after retinal ischemia in vivo. FIG. 47: Stimulus intensity plots of a-(A) and b-waves (B) were measured at baseline and at 8 days post ischemia. MSC-EVs, PBS, or MSC medium depleted of EVs (EV depleted medium) were injected 24 h after ischemia into the vitreous humor of both eyes (right eye was ischemic and left eye was non ischemic control), as described in the methods section. FIG. 48: (C) Representative ERG traces from ischemic retinae injected with PBS, MSC-EVs and medium depleted of EVs respectively; for brevity, only one set of representative traces, from ischemic eyes, per group is shown. The scale bars for amplitude (Y-axis, μV) and latency (X-axis, ms) appear in the top right of each representative ERG panel. N=11-13 rats, for MSC-EVs or PBS; N=6 for MSC-EV depleted medium. *=P<0.05 for ischemic+MSC-EVs vs ischemic+PBS, #=P<0.05 for medium depleted of MSC-EVs+ischemic vs MSC-EVs+ischemic.

FIGS. 49 and 50. MSC-EVs attenuated ischemia-induced apoptosis (TUNEL, terminal deoxynucleotidyl transferase-mediated dUTP nick end labeling assay) in ischemic retinae in vivo. FIG. 49: Representative immuno-histochemical images of TUNEL in retinal cryosections (7 μm) demonstrating MSC-EV-mediated reduction in TUNEL cells in ischemic retina compared to PBS injected ischemic. TUNEL; DAPI; fluorescently labeled MSC-EVs. In these experiments, the retinal cryosections were taken from retinae at 24 h after intravitreal injection of MSC-EVs or PBS, which was 48 h after ischemia. TUNEL cells are seen in the RGC layer (grey arrows, upper right quadrant), and in the inner (INL) and outer nuclear layers (ONL) (white arrows, upper right quadrant). IPL=inner plexiform layer. Note that aggregates of MSC-EVs (grey arrows, lower quadrants) are present in the retinal ganglion cell (RGC) layer in EV ischemia (bottom right panel), and in the vitreous in EV control (bottom left panel). FIG. 50: Graphical representation of TUNEL cells in retinal ganglion cell layer, inner nuclear layer, outer nuclear layer, and total nuclei in retina, with data shown on Y-axis as TUNEL cell/20×field, mean±SD. TUNEL was counted in all four groups (PBS control, MSC-EV control, PBS+ischemia and MSC-EVs+ischemia) by blinded observers. MSC-EVs attenuated TUNEL in ischemic retinae, and there was no significant increase in TUNEL in normal eyes injected with MSC-EVs (“EV control”) except in the RGC layer. N=4 rats per group; *=P<0.05 for PBS non-ischemic, or MSC-EV non-ischemic vs MSC-EV ischemic; #=P<0.05 for PBS ischemic vs MSC-EV ischemic. **=P<0.05 for MSC-EV non-ischemic vs PBS non-ischemic.

FIGS. 51 and 52. MCS-EVs attenuated neuro-inflammation and caspase 3 activation after retinal ischemia in vivo. FIG. 51A: Representative Western blots for TNFα, IL-6 and cleaved caspase 3. β-Actin was used as the loading control. FIG. 51B, FIGS. 52C and D: Quantitative bar graphs for Western blots illustrating the significant MSC-EV-mediated amelioration of ischemia-induced increases in levels of inflammatory mediumtors (IL-6, TNFα), and apoptosis (cleaved caspase 3) in rats injected with intravitreal MSC-EVs 24 h after ischemia. There was no significant change in levels of IL-6, TNFα, or caspase 3 in MSc-EV injected normal eyes compared to PBS injected normal eyes. Retinal samples were collected 48 h after ischemia, which was 24 h after MSC-EV or PBS injection. N=10 rats per group, *=P<0.05 control non-ischemic vs ischemic, #=p<0.05 PBS+ischemic vs MSC-EV+ischemic.

FIGS. 53 and 54. In vivo live imaging of intra-vitreally injected fluorescent MSC-EVs. FIG. 53: Uptake of MSC-EVs intro vitreous and retina of normal and ischemic eyes was imaged in real time by in vivo fundus imaging for a time course of four weeks (days 1 and 3, weeks 1, 2, and 4), using a Phoenix Micron IV. The control non-ischemic eyes are on the left and ischemic on the right in each of the two columns in (A). Fluorescent MSC-EVs were present for up to 4 weeks after injection into the vitreous humor. Concentration of the MSC-EVs at the sites of injection into the vitreous and in the needle track likely explain the intense fluorescence in the day 1 and 3 images. FIG. 54: Graph representing binding of fluorescently labeled MSC-EVs to 50 μg of isolated humor coated to 96-well assay plates. The binding of MSC-EVs to the vitreous humor was saturable. Data point represent mean±SD (n=6 experiments) of normalized fluorescence intensity.

FIGS. 55, 56, and 57. Uptake and distribution of MSC-EVs by normal and ischemic retinae in vivo. Flat mount confocal microscopic imaging of retinae injected with fluorescent MSC-EVs and stained with retinal markers anti-Brn-3a for retinal ganglion cells (RGCs), anti-Iba-1 for microglia and nuclei (DAPI). FIG. 55: Representative images displayed for days 1, 3 and 7 for PBS-injected control (I) and ischemic (II) retinae. FIG. 56: Representative images displayed for days 1, 3, and 7 for MSC-EV injected control (III) and ischemic retinae. For each group a low magnification image is presented in one channel indicating the overview of the flat mount. The square white box indicates the representative area shown under high magnification. Higher magnification images (63×) are provided in all channels followed by a merged image for days 1 (A to E), 3 (F to J) and 7 (K to O). Comparing (III) and (IV), enhanced MSC-EV uptake can be seen in the ischemic (IV) compared to the normal retina (III), along with enhanced co-localization with the activated microglia. The composite images (E, J, and O) for each group show co-localization of MSC-EVs and microglia (white arrows in panel IVE), and Brn3a (white dots, shown by grey arrows in panel IVE), indicating that MSC-EVs were taken up by both RGCs and microglia after intravitreal administration. Grey arrows in panels HD and IVD show the greater amoeboid shape as opposed to ramified microglia indicating greater activation of microglia in ischemia-PBS injected compared to ischemia-MSC-EV injected retinae. N=3 per time point. FIG. 57: The uptake of MSC EVs by RGCs is further illustrated in (B), that are representative digital magnification of retinal flat mount images in (A) illustrating co-localization of MSC-EVs and distribution by specific retinal cell type in MSC-EV injected control and ischemic retinae. Grey arrows in the top panel of (b) point to RGCs co-localized with MSC-EVs and dark arrows in the bottom panel of (b) point to MSC-EVs with microglial cells.

FIG. 58. High magnification confocal imaging of retinal flat mounts shows that retinal neurons and retinal ganglion cells take up MSC-EVs, and that ischemia increases uptake. Top panel shows control, non-ischemic retina, and bottom panel shows ischemic retina. Retinal flat mounts of non-ischemic eyes injected with labeled MSC-EVs, stained for (A) DAPI, (B) EVs alone, (C) Beta-tubulin III alone (βT3), and (D) Brn-3a alone, (E) EVs+βT3 and (F) EVs Brn-3a. βT3 stains only neurons and their axonal or dendritic projection. These flat mounts are from retinas harvested 24 h after injection of MSC-EVs, which was 48 h after ischemia. Arrows in (F) indicate the presence of EVs within the cell body of the retinal ganglion cells (Brn-3a stains only the nuclei of RGCs). Note that the majority of cells in (B), (E), and (F) show punctate staining indicating that EVs were taken up by the cells. White arrows in (E) show the co-localization between the MSC-EVs and the retinal neuron cell bodies. White arrowheads mark the axonal or dendritic projection of the retinal neurons, and the presence therein of MSC-EVs (E).

FIG. 59. Differential miRNA reads in various groups of exosomes. The third column represents the total number of the raw reads in the original input file. The fourth column represents the numbers of the reads which can be mapped to the miRNA reference genome. The fifth column represents the percentage of the reads which can be mapped to the miRNA reference genome comparing to the total number of the short reads. The sixth column represents the number of the reads which can be mapped to the miRNA reference genome, after the PCR duplicates have been removed. Also, a big portion of the reads which can be mapped as miRNA are PCR duplicates. In the second tab (Raw count), the number of the short reads which can be mapped as miRNA are further classified by each miRNA. Each column represents a sample. Each row represents one miRNA. In each sample, the number of reads for each miRNA were normalized by the library size (number of the total reads in the library).

FIG. 60. Table of top miRNA reads for various exosome sample populations.

FIG. 61. Schematic for reaction assembling alginate peptide modification.

FIG. 62. Schematic for reaction assembling methacrylated alginate.

FIG. 63. Graph showing hMSC Regular exosome binding and releasing profiles on the coated peptides—volume of exosomes study. Binding and release of MSC exosomes to various collagen and fibronectin derived peptides was assayed.

FIG. 64. Graph showing hMSC Regular Exosome binding and releasing profiles on the coated peptides—time study. Binding and release of MSC exosomes to various collagen and fibronectin derived peptides was assayed.

FIG. 65. Graph showing hMSC exosome release from photocrosslinkable alginate hydrogels.

FIG. 66. Graphs showing hMSC exosome release profile from photocrosslinkable alginate hydrogels with and without RGD.

FIG. 67. hMSC Regular Exosome loaded in the alginate hydrogel (AMARGD), 4 hrs after hMSC seeded on top of the hydrogel. Staining is for nuclei (DAPI).

FIG. 68. hMSC Regular Exosome loaded in the alginate hydrogel (AMARGD), 4 hrs after hMSC seeded on top of the hydrogel. Staining is for exosomes.

FIG. 69. hMSC Regular Exosome loaded in the alginate hydrogel (AMARGD), 4 hrs after hMSC seeded on top of the hydrogel. Staining shown is merged, for both nuclei and exosomes.

FIG. 70. hMSC Regular Exosome loaded in the alginate hydrogel (AMARGD), 3 days after hMSC encapsulated in the hydrogel—merged staining, actin and exosomes.

FIG. 71, 71A. hMSC Regular Exosome loaded in the alginate hydrogel (AMARGD), 3 days after hMSC encapsulated in the hydrogel. FIG. 71: top, exosomes; bottom, actin. FIG. 71A: merge of exosomes and actin.

FIG. 72. Exosome release kinetics for various 3-D printed hydrogels.

FIG. 73. hMSC BMP2 Exosomes loaded on alginate hydrogel in vitro experiment—contactless experiment. The top figure shows the configuration for the experiment. The bottom shows the fold change at 3 days and 5 days for the factors indicated.

FIG. 74. hMSC BMP2 Exosomes loaded on alginate hydrogel in vitro experiment—contact experiment. The top figure shows the configuration for the experiment. The bottom shows the fold change at 3 days and 5 days for the factors indicated.

FIG. 75. BMP2 Exo mediated bone regeneration. Representative μCT images showing regeneration of bone in 5 mm calvarial defects that were treated with plain collagen sponge (Control), collagen sponge containing control EVs (Ctrl. Exo), collagen sponge containing BMP2 (BMP2 GE) and collagen sponge containing BMP2 Exo at 4, 8 and 12 weeks post wounding.

FIG. 76. hMSC BMP2 Exosomes loaded on alginate hydrogel in vivo experiment. Similar to FIG. 75, with calvarial defects treated with exosomes on alginate hydrogels. Results for 4 and 8 weeks are shown.

FIG. 77. List of miRNA primer sequences used to measure expression levels in exosomes.

FIG. 78. Isolation and characterization of EVs. A) Representative NTA plots of EVs isolated from naïve, osteogenic, chondrogenic and adipogenic differentiated HMSCs. Note that the size distribution falls within the range of extracellular vesicles characterized as EVs. B) Representative transmission electron microscopy images of the EVs isolated from naïve, osteogenic, chondrogenic and adipogenic HMSCs. C) Immunoblot of protein isolates from EVs from naïve, osteogenic, chondrogenic and adipogenic HMSCs showing the presence of CD63 exosomal marker protein. D) Immunoblot indicating the presence of EV marker CD9 in the EV protein isolates mentioned above.

FIG. 79. Endocytosis of HMSC EVs by HMSCs. A) Graphical representation of dose-dependent and saturable endocytosis of fluorescently labeled HMSC EV by naïve HMSCs. Data points represent mean fluorescence (n=6)+/−SD. The EV volume/particle number was standardized as described under the methods section. B) Graph showing the dose dependent inhibition of HMSC EV endocytosis after pre-treatment of the EVs with heparin to block interaction with the cell surface HSPGs. Data represent mean percentage fluorescence with respect to control+/−SD (n=6). * represents statistical significance (P<0.05) with respect to control by student's t-test. C) Graph showing the reduction in HMSC endocytosis after disruption of target cell membrane cholesterol with varying doses of MBCD. Data is presented as mean percentage fluorescence with respect to control+/−SD (n=6). * represents statistical significance (P<0.05) with respect to control by student's t-test. D) Representative confocal micrograph depicting the endocytosed fluorescently labeled HMSC EVs within target HMSCs after 1 hour of incubation at 37° C. E) Representative confocal micrograph indicating the abrogation of MSC EV endocytosis when the experiment is performed at 4° C. F) Representative confocal micrograph showing that pre-treatment of EVs with heparin blocks MSC EV endocytosis. G) Representative confocal micrograph of MSC EV endocytosis after pre-treatment of the cells with 2 mM RGD peptide to block cell surface integrins. In images D, E, F and G EVs, tubulin, and nuclei are labeled. H) Confocal micrograph showing colocalization of endocytosed MSC EVs with caveolin1. I) Confocal micrograph showing the absence of co-localization between endocytosed EVs and clathrin.

FIG. 80. Endocytosis of EVs isolated from differentiated HMSCs. A) Representative confocal micrographs of fluorescently labeled EVs isolated from control (naïve), osteogenic, adipogenic and chondrogenic HMSCs endocytosed by naïve HMSCs. In all images, EVs and blue represents DAPI nuclear stain. Scale bar represents 10 μm in all images. B) Graph showing dose dependent and saturable endocytosis of EVs isolated from osteogenic, chondrogenic and adipogenic HMSCs by naïve HMSCs. Data points represent mean percentage fluorescence with respect to the highest concentration+/−SD (n=6). Note the absence of any significant difference in endocytosis between EVs isolated from the three lineages.

FIG. 81. EV mediated lineage-specific differentiation of HMSCs in vitro. A, B and C represent fold changes in gene expression levels of representative marker genes for osteogenic, chondrogenic and adipogenic differentiation of HMSCs after treatment of naïve HMSCs for 72 hours with the EVs isolated from respectively differentiated HMSCs. The data are presented as mean fold change with respect to control (n=4). The data presented also shows the statistical significance in the form of P value for each data point obtained by student's t-test in comparison with the respective controls. The data represents fold change for genes unique to the specific lineage. No significant change was observed in the represented genes upon treatment with EVs from other lineages.

FIGS. 82 and 83. EV mediated lineage-specific differentiation of HMSCs in vivo. A) Confocal micrographs representing immunohistochemical staining for the presence of phosphorylated proteins (pSTT) by staining for phosphorylated serines, threonines and tyrosines and DMP1 in control and osteogenic EV treated subcutaneous explant tissue sections. Note the increase in the expression levels of phosphorylated proteins and DMP1 in the osteogenic EV treated group. B) Confocal micrographs representing immunohistochemical staining for type II collagen and the anti-angiogenic factor PEDF in control and chondrogenic EV treated subcutaneous explant tissue sections. Note the increase in the expression levels of both proteins in the chondrogenic EV treated group. C) Confocal micrographs representing immunohistochemical staining for PPARγ and caveolin 1 (cav-1) in control and adipogenic EV treated subcutaneous explant tissue sections. Note the increase in the expression levels of PPARγ and the decrease in the expression levels of caveolin1 in the chondrogenic EV treated group. Additionally, also note the presence of fat globule-like morphology in the PPARγ positively stained cells.

FIG. 84. Characterization of BMP2 OE HMSCs and BMP2 EV. A) Graph representing the fold change in the expression levels of BMP2 gene in vector control and BMP2 OE HMSCs with respect to untreated controls. Data represent mean fold change+/−SD of three independent cultures. B) Representative images of alizarin red stained culture dishes of control, vector control and BMP2 OE HMSCs after 7 days of culture in osteogenic differentiation media. Note the increase in calcium deposits in the BMP2 OE HMSC group. C) Representative TEM image of BMP2 EV immunolabeled for CD63 (10 nm gold dots). D) Representative NTA plot of BMP2 EV indicating exosomal size distribution. E) Graphical representation of dose-dependent and saturable endocytosis of fluorescently labeled BMP2 EVs by naïve HMSCs. Data points represent mean fluorescence (n=6)+/−SD. The EV volume was standardized as described under the methods section.

FIGS. 85 and 86. BMP2 EVs potentiate the BMP2 signaling cascade. A) Fold change in osteogenic gene expression (w.r.t untreated control) after HMSCs were treated with BMP2 EVs for 72 hrs. * Represents statistical significance w.r.t untreated control group (n=4). B) Representative western blot showing phosphorylated SMAD 1/5/8 (red lanes to the left) and tubulin (green to the right) after treatment of HMSCs with rhBMP2, Control EVs and BMP2 EVs. Note the increase in the band intensity for phosphorylated SMAD 1/5/8 after treatment with positive control BMP2 and with BMP2 EVs. The graph below shows percentage increase in luciferase activity of the SMAD 1/5 specific reporter. Note the increase in activity after treatment with BMP2, BMP2 EVs and the combination of BMP2 and BMP2 EVs. * Represents statistical significance w.r.t untreated control and # represents statistical significance w.r.t the rhBMP2 treated group (n=4 for all groups). C) Dual immunoblot for BMP2 (red) and CD63 (green) showing the presence of BMP2 in the EV-depleted conditioned medium from the BMP2 OE cells but not in the EV protein isolates of the control cell conditioned medium. CD63 was observed in the EV protein isolates only. D) Table listing the mean fold change (n=4) in the expression levels of miRNA that bind to the 3′UTR of SMAD7 and SMURF1. miR 3960 is a pro-osteogenic miRNA that remained unchanged and is used as a control to show pathway specific increase in EV miRNA composition. P value was calculated using student's t-test.

FIGS. 87 and 88. BMP2 Exo mediated bone regeneration. A) Representative μCT images showing regeneration of bone in 5 mm calvarial defects that were treated with plain collagen sponge (Control), collagen sponge containing control EVs (Ctrl. Exo), collagen sponge containing BMP2 (BMP2 GF) and collagen sponge containing BMP2 Exo at 4, 8- and 12-weeks post wounding. The arrow in the 12 week BMP2 GF group shows ectopic bone formation. B) Volumetric quantitation of the μCT data expressed as percentage bone volume regenerated with mineralized tissue (n=6 defects per group per time point). * represents statistical significance (P<0.05, student's t-test) with respect to the collagen control group (no EV). # represents statistical significance (P<0.05, student's t-test) between the control EV and BMP2 GF group. ## represents statistical significance (P<0.05, student's t-test) between the BMP2 EV and control EV groups.

FIG. 89. Histological evaluation of calvarial defects. Images are representative light microscopy images of H&E stained demineralized calvarial samples of defects treated with plain collagen sponge (Control), collagen sponge containing control EVs (Ctrl. Exo), collagen sponge containing BMP2 (BMP2 GF) and collagen sponge containing BMP2 Exo after 4, 8 and 12 weeks post wounding. The black arrows in the images point to regenerated bone tissue. The yellow arrows in the BMP2 GF group point to fat deposits within the regenerated bone. Scale bar represents 200 μm in all images.

FIGS. 90 and 91. BMP2 and BSP IHC. Images represent the expression levels of BMP2 and BSP in the calvarial sections from the different groups after 4 weeks. Note the increase in the expression levels of both proteins in the rhBMP2 treated (BMP2 GF) and BMP2 EV treated groups.

FIGS. 92 and 93. DMP1 and OCN IHC. Images represent the expression levels of DMP1 and OCN in the calvarial sections from the different groups after 4 weeks. Note the increase in the expression levels of both proteins in the BMP2 EV treated group compared to the control groups.

### DETAILED DESCRIPTION

Provided herein are compositions, methods, and systems for making and using engineered exosomes and treating various disorders, such as bone or neuronal disorders, thereby.

Before the disclosed processes and materials are described, it is to be understood that the aspects described herein are not limited to specific embodiments, and can vary. It also will be understood that the terminology used herein is for the purpose of describing particular aspects only and, unless specifically defined herein, is not intended to be limiting.

In view of the present disclosure, the methods and compositions described herein can be configured by the person of ordinary skill in the art to meet a particular desired need. In general, the disclosed materials and methods provide advances over the prior art regarding exosome compositions and their use in treatment of various diseases and disorders.

Tissue engineering approaches for regenerating tissues such as bone, cartilage, skin, muscle and liver utilize growth factors and morphogens to enable stem cell differentiation. This approach is fraught with challenges such as dosage, ectopic activity, delivery and immunological complications limiting clinical use and translation. Engineered exosomes can be used as an alternative to growth factors to induce/enhance tissue regeneration. As disclosed herein, functionality and target specificity has been engineered into exosomes to generate Functionally Activated Targeted Exosomes (FATE) for tissue engineering and regenerative medicine applications.

### Therapeutic Applications

The compositions of the disclosure as provided herein can be used in treatment of various diseases and disorders. Thus, in one aspect, the disclosure provides methods of treating bone diseases or disorders. Such methods include administering the compositions of the disclosure as described herein to a subject in need of treatment. Bone diseases that can be treated with the methods of the disclosure, for example, include but are not limited to bone defect, damage, and fracture, including for dentoalveolar indications. In certain embodiments, the bone disease is a bone defect, damage, or fracture.

In another aspect, the disclosure provides methods for treatment of neurological diseases or disorders. Such methods include administering the compositions of the disclosure as described herein to a subject in need of treatment. Neurological diseases or disorders that can be treated with the methods of the disclosure, for example include, but are not limited to, stroke/ischemia, loss of neuronal function, neuronal cell death and severed nerves. In certain embodiments, the neurological disease is stroke/ischemia. In some embodiments, the disclosure provides method for treating a disease or disorder in an individual, comprising administering a therapeutically effective amount of the composition of any of claims 1-42 to the individual in need thereof. In some embodiments, the disease or disorder is a bone disorder. In some embodiments, the disease or disorder is bone defect, fracture, or a dentoalveolar disorder.

In some embodiments, the disease or disorder is a neurological disorder. In some embodiments, the disease or disorder is ischemia, loss of neuronal function, neuronal cell death, or severed nerves. In some embodiments, the composition is administered by injection.

In some embodiments, the composition is administered by implantation. In some embodiments, the composition is administered by 3D-printed material. In some embodiments, the dosage is 1×106 to 1×1012 exosomes per unit mm3 of graft, tissue, patch or injection volume or ointment.

In some embodiments, the disclosure provides a method for treating an eye disorder in an individual comprising delivering a composition of isolated exosomes to vitreous humour of the individual, wherein the exosomes are enriched in regenerative factors endogenous to stem cells.

### Administration of the Compositions

In some embodiments, dosages of 1×10⁶ to 1×10¹² exosomes per unit mm³ of graft, tissue, patch, or injection volume are administered. Exosome dosage may be determined by the volume of the area to be treated (i.e. the size of the graft or tissue), or by the volume of the composition to be administered (i.e. the size of the patch, or the volume to be injected).

In some embodiments, exosome compositions are administered as a single bolus. In other embodiments, multiple administrations can be required. For example, exosomes can be administered every other month, once per month, twice per month, one per week, week, several times per week (e.g., every other day), or once per day, depending upon, among other things, the mode of administration, the specific indication being treated, and the judgment of the prescribing physician.

Various methods of administering exosomes are contemplated. Exosome compositions disclosed herein can take a form suitable for virtually any mode of administration, including, for example, topical, ocular, oral, buccal, systemic, nasal, injection, transdermal, rectal, vaginal, etc., or a form suitable for administration by inhalation or insufflation. In some embodiments, exosome compositions are administered by injection. Injection is a technique for delivering drugs by parenteral administration, including subcutaneous, intramuscular, intravenous, intraperitoneal, intracardiac, intraarticular, and intracavernous injection, all of which are contemplated by the present disclosure.

In some embodiments, exosome compositions are administered by implantation, i.e. through use of an implant. An implant is a medical device manufactured to replace a missing biological structure, support a damaged biological structure, or enhance an existing biological structure. Implant surfaces that contact a body or portion thereof can be made of a biomedical material such as titanium, silicone, or apatite depending on what is the most functional. An implant can be made of a bioactive material.

### Compositions of the Disclosure

In general, the present disclosure concerns compositions and methods of making and using isolated exosomes. As used herein, an isolated exosome is an exosome that is physically separated from its natural environment. For example, an isolated exosome may be physically separated, in whole or in part, from tissue or cells within which it naturally exists, including MSCs, In some embodiments of the disclosure, a composition of isolated exosomes may be free of cells such as MSCs or free or substantially free of media.

In some embodiments, the disclosure provides compositions comprising isolated engineered exosomes from mesenchymal stem cells (MSCs), each exosome comprising at least one factor that is: an osteoinductive factor, a neuronal regeneration factor, an immunomodulatory factor, an extracellular matrix binding factor, or a combination thereof, wherein the at least one factor is present at a higher amount in the engineered exosome than the amount present in a naturally occurring cell-derived exosome. The exosomes of the disclosure are also engineered. In some embodiments, the exosomes are engineered in vitro. The exosomes can be engineered through genetic modification of a parental cell that gives rise to the exosomes. In some embodiments, exosomes are engineered by exposing parental cells to a stimulus, for instance, a particular compound or molecule in the culture medium. In some embodiments, the stimulus can be a deficit of a necessary element (i.e., oxygen).

### Factors

In some embodiments, the engineered exosomes comprise one or more factors at a higher level or concentration than the level or concentration present in a naturally occurring cell-derived exosome. A factor can be a molecule, for instance, a protein, peptide, nucleic acid, lipid, or carbohydrate. A factor can be a small molecule or a macromolecule. A naturally occurring cell-derived exosome is an exosome that has arisen without human manipulation of the parent cell or the exosome itself. If a naturally occurring exosome has been isolated, it has been isolated using means that do not change any of its characteristics.

In some embodiments, the one or more factors is one or more microRNAs. A microRNA (miRNA, or miR as named) is a small non-coding RNA molecule (containing about 22 nucleotides) found in plants, animals and some viruses, that functions in the regulate gene expression in various biological processes and signaling pathways. MicroRNAs are abundant in many mammalian cells and are known to target approximately 60% of genes. They also play a key role in various pathologies ranging from metabolic diseases to cancer. miRNA can impact biological function as either suppressors of gene expression (when their expression levels are enhanced, for instance, in disease state or through human intervention) or upregulators of gene expression (when their expression levels are reduced). A microRNA can be tissue specific or ubiquitously expressed. In some embodiments of the current disclosure, the compositions comprise one or more of let 7a, miR 218, miR 9-5p, miR 19a-3p, mir 30a-5p, miR 212-5p, miR 323-5p, miR 15a, miR 15b, miR 16, miR 424 and miR 497 at a higher level than the level present in a naturally occurring cell-derived exosome. In some embodiments, the one or more factors is a member of a particular molecular pathway (“pathway member”). A pathway member is a molecule for which activity or amount in a given cell is responsive to the activity or amount of the named molecule defining the pathway.

In some embodiments, the one or more factors comprise osteoinductive factors. Osteoinductive factors are those that promote or facilitate development or healing of bone tissue. These factors can be present in the exosomes, and in addition, they can be used to engineer parental cells to yield potent exosomes (i.e. these factors can be a “stimulus”). Osteoinductive factors include, but are not limited to, transforming growth factors (TGFs), bone morphogenetic proteins (BMPs), fibroblast growth factors (FGFs), insulin-like growth factors (IGFs), platelet-derived growth factors (PDGFs), osterix (OSX), and RUNX. A microRNA, such as let 7a, miR 218, miR 9-5p, miR 19a-3p, mir 30a-5p, miR 212-5p, miR 323-5p, miR 15a, miR 15b, miR 16, miR 424 and/or miR 497, can be an osteoinductive factor.

In some embodiments, the one or more factors comprose neuronal regeneration factors. Neuronal regeneration factors are those that promote or facilitate development or healing of neuronal tissue. These factors can be present in the exosomes, and in addition, they can be used to engineer parental cells to yield potent exosomes (i.e. these factors can be a “stimulus”). Neuronal regeneration factors include, but are not limited to, c-Jun, activating transcription factor-3 (ATF-3), SRY-box containing gene 11 (Sox11), small proline-repeat protein 1A (SPRR1A), growth-associated protein-43 (GAP-43) and CAP-23. A microRNA, such as miR 424, can be a neuronal regeneration factor.

In some embodiments, the one or more factors comprise immunomodulatory factors. Immunomodulatory factors are those that influence aspects of the immune system, for instance, macrophage populations. These factors can be present in the exosomes, and in addition, they can be used to engineer parental cells to yield potent exosomes (i.e. these factors can be a stimulus”). Immunomodulatory factors include, but are not limited to cytokines, interferon, interleukin, antigens, and growth factors. A microRNA, such as miR-9-5p, miR19a-3p, miR-30a-5p, miR-212-5p, and/or miR-323-5p, can be an immunomodulatory factor.

In some embodiments, the composition comprises isolated engineered exosomes from mesenchymal stem cells (MSCs), each exosome comprising at least one factor that is: an osteoinductive factor, a neuronal regeneration factor, an immunomodulatory factor, an extracellular matrix binding factor, or a combination thereof, wherein the at least one factor is present at a higher amount in the engineered exosome than the amount present in a naturally occurring cell-derived exosome. In some embodiments, the at least one osteoinductive factor is present in the engineered exosome at a higher amount than the amount present in a naturally occurring cell-derived exosome. In some embodiments, the at least one osteoinductive factor comprises let 7a, miR 218, miR 9-5p, miR 19a-3p, mir 30a-5p, miR 212-5p, and miR 323-5p. In some embodiments, the at least one osteoinductive factor comprises let 7a. In some embodiments, the amount of let 7a in the engineered exosomes is at least 10-fold higher than the amount of let 7a in the naturally occurring cell-derived exosomes. In some embodiments, the amount of let 7a in the engineered exosomes is at least 35-fold higher than the amount of let 7a in the naturally occurring cell-derived exosomes. In some embodiments, the at least one osteoinductive factor comprises miR 218. In some embodiments, the amount of miR 218 in the engineered exosomes is at least 10-fold higher than the amount of miR 218 in the naturally occurring cell-derived exosomes. In some embodiments, the amount of miR 218 in the engineered exosomes is at least 45-fold higher than the amount of miR 218 in the naturally occurring cell-derived exosomes. In some embodiments, the at least one osteoinductive factor comprises one or more of miR-9-5p, miR-19a-3p, miR-30a-5p, miR-212-5p, miR-323-5p, miR 15a, miR 15b, miR 16, miR 424, and miR 497. In some embodiments, the at least one osteoinductive factor is an miRNA that positively regulates at least one RUNX2 and/or OSX pathway member. In some embodiments, the amount of the one or more osteoinductive factors in the engineered exosomes is at least 3-fold higher than the amount of any of the one or more osteoinductive factors in the naturally-occurring cell-derived exosomes. In some embodiments, the engineered exosomes comprise at least one immunomodulatory factor, wherein the composition decreases the ratio of pro-inflammatory M1 macrophages to anti-inflammatory M2 macrophages relative to the ratio demonstrated by the activity of naturally occurring cell-derived exosome.

In some embodiments, the at least one immunomodulatory factor comprises miRNAs that downregulate at least one NF [symbol] B, SOCS3, and/or IRF-5 pathway member. In some embodiments, the at least one immunomodulatory factor comprises miRNAs that upregulate at least one LXR-alpha, STAT6, and/or P13/Akt pathway member. In some embodiments, the ratio of pro-inflammatory M1 macrophages to anti-inflammatory M2 macrophages is less than the ratio present in non-healing wound of bone or neuronal tissues.

In some embodiments, the engineered exosomes comprise at least one neuronal regeneration factor, wherein the at least one neuronal regeneration factor is present at a higher amount than the amount present in a naturally occurring cell-derived exosome. In some embodiments, the at least one neuronal regeneration factor comprises miR 424. In some embodiments, the amount of miR 424 in the engineered exosomes is at least 10-fold higher than the amount of miR 424 in the naturally occurring cell-derived exosome. In some embodiments, the amount of miR 424 in the engineered exosome is at least 100-fold higher than the amount of miR 424 in the naturally occurring cell-derived exosomes.

In some embodiments, the engineered exosomes comprise at least one extracellular matrix binding factor, wherein the at least one extracellular matrix binding factor is present in the engineered exosome at a higher amount than the amount present in a naturally occurring cell-derived exosome. In some embodiments, the at least one extracellular matrix binding factor comprises integrin α5. In some embodiments, the amount of integrin α5 in the engineered exosome is at least 1.5-fold higher than the amount of integrin α5 present in a naturally occurring cell-derived exosome. In some embodiments, the at least one extracellular matrix binding factor increases the binding affinity or rate to one or more components of the extracellular matrix and/or extracellular matrix-derivative peptides in a dose-dependent manner. In some embodiments, the components of the extracellular matrix comprise one or more of proteins (e.g., collagen, elastin, fibrin etc.), glycoproteins (e.g., fibronectins, laminins, etc.), proteoglycans, and polysaccharides (e.g., hyaluronic acid, alginate, heparin functionalized with extracellular matrix proteins or extracellular matrix-derivative peptide motifs, PLA functionalized with extracellular matrix proteins or extracellular matrix-derivative peptide motifs, and PGA functionalized with extracellular matrix proteins or extracellular matrix-derivative peptide motifs). In some embodiments, the one or more components of extracellular matrix comprises one or more of COL1 and FN1.

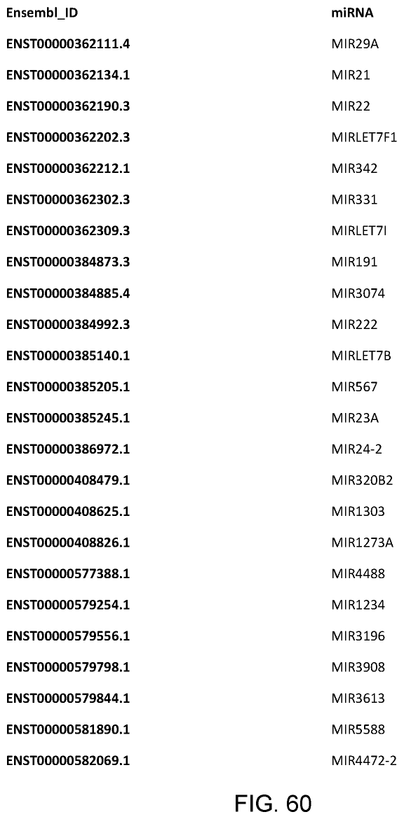

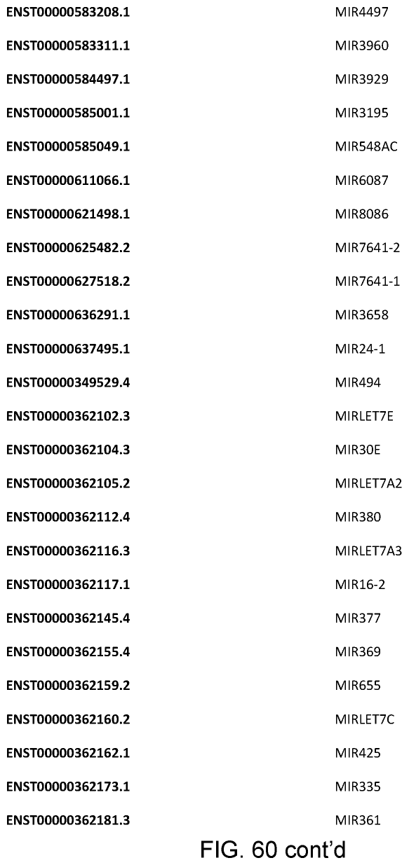

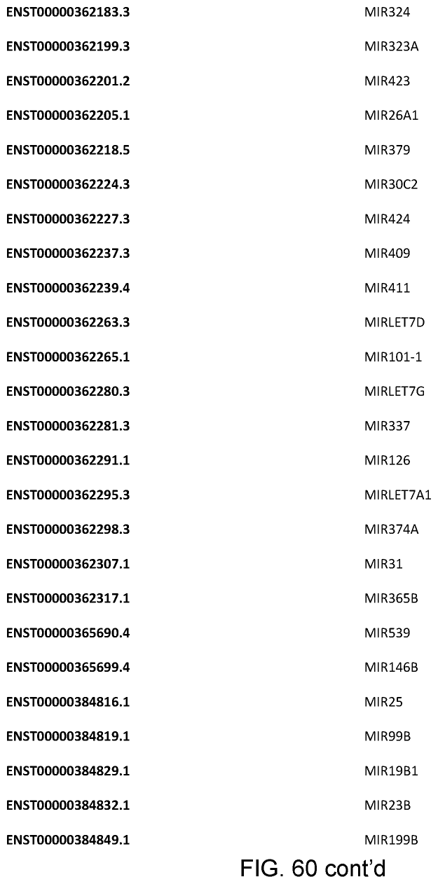

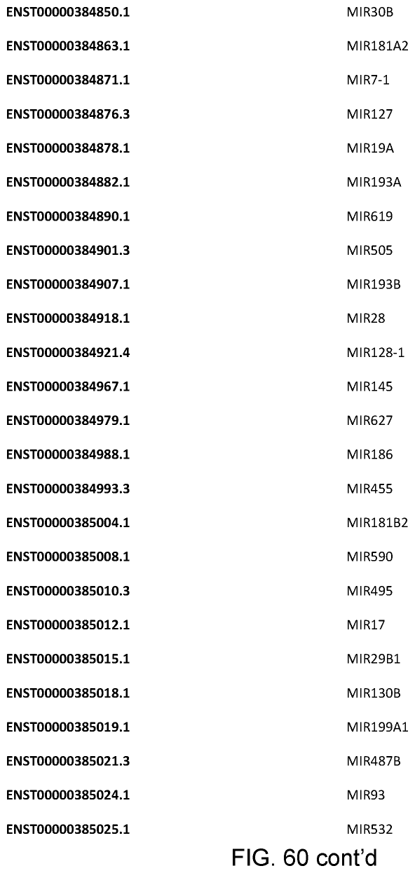

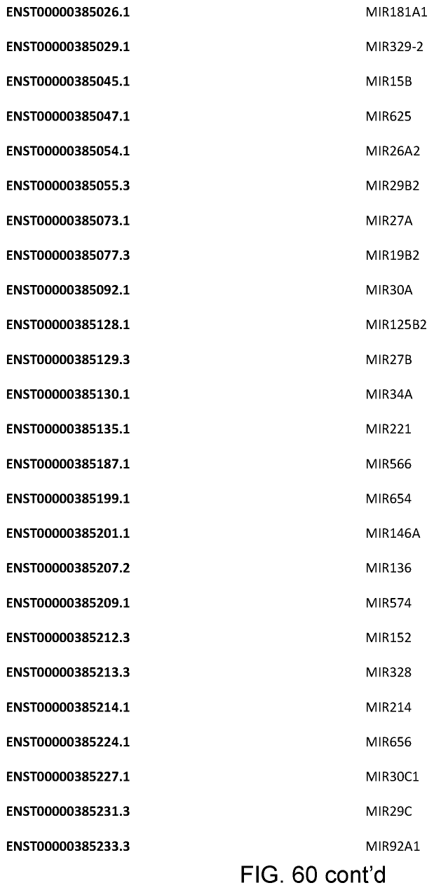

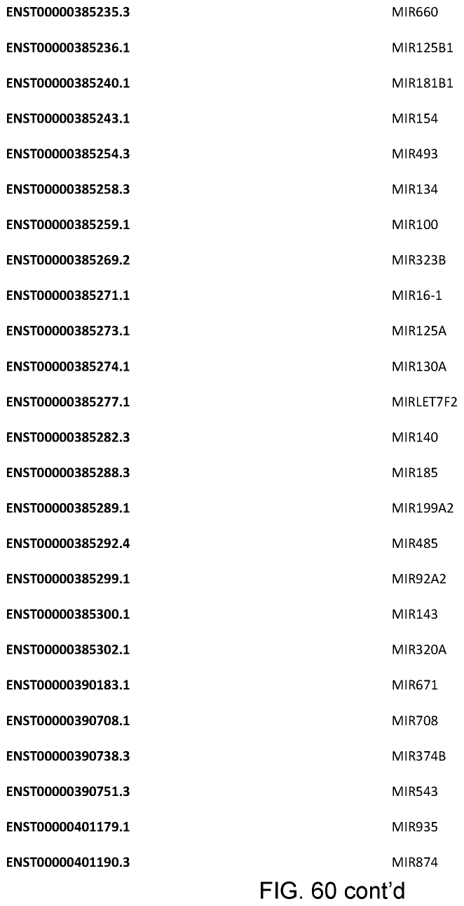

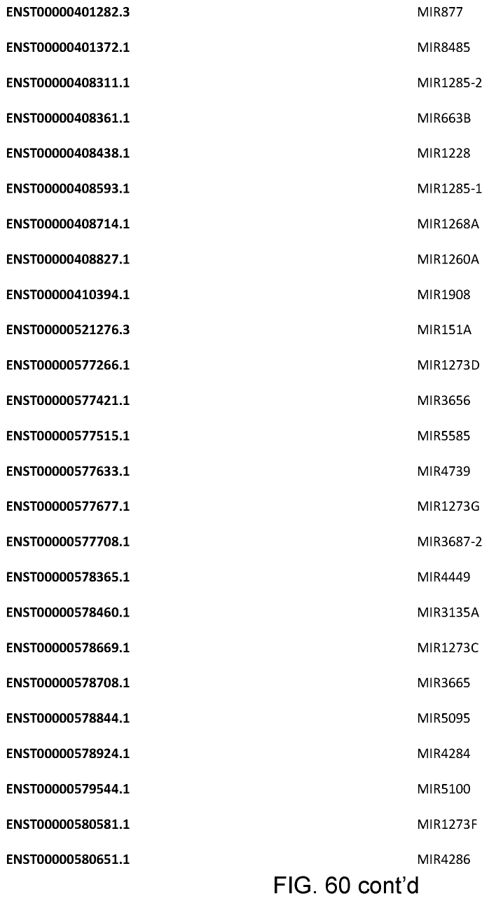

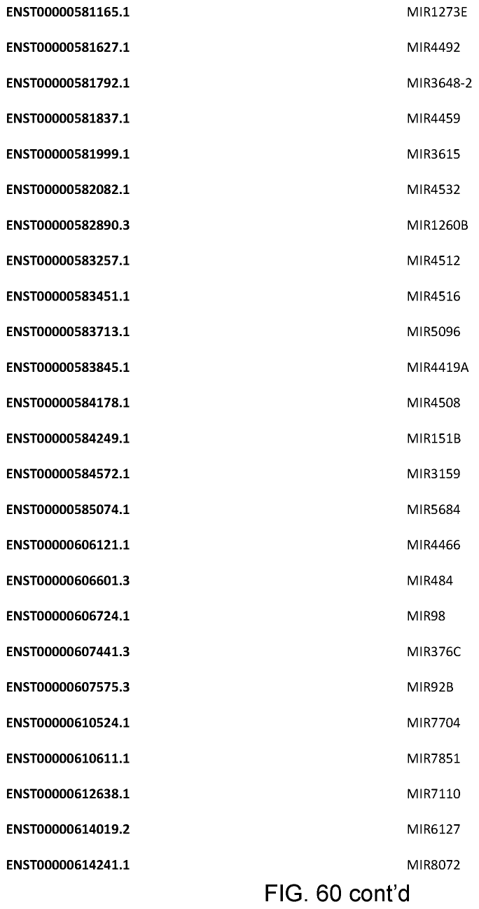

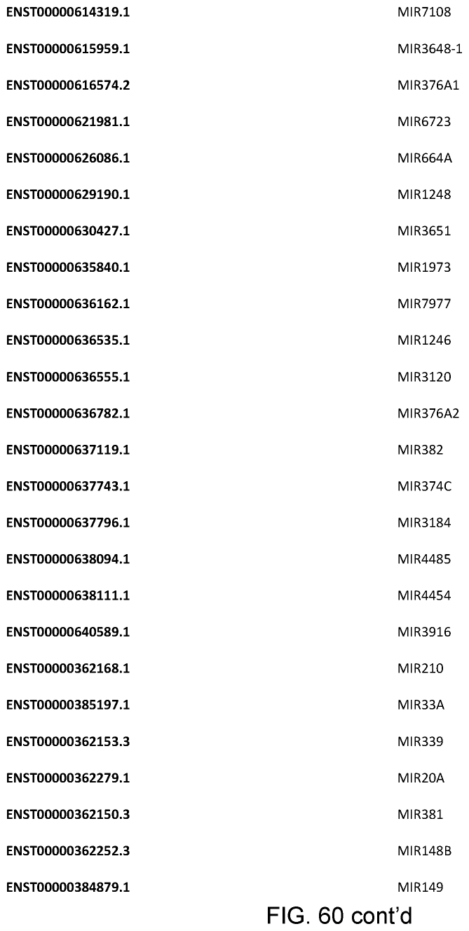

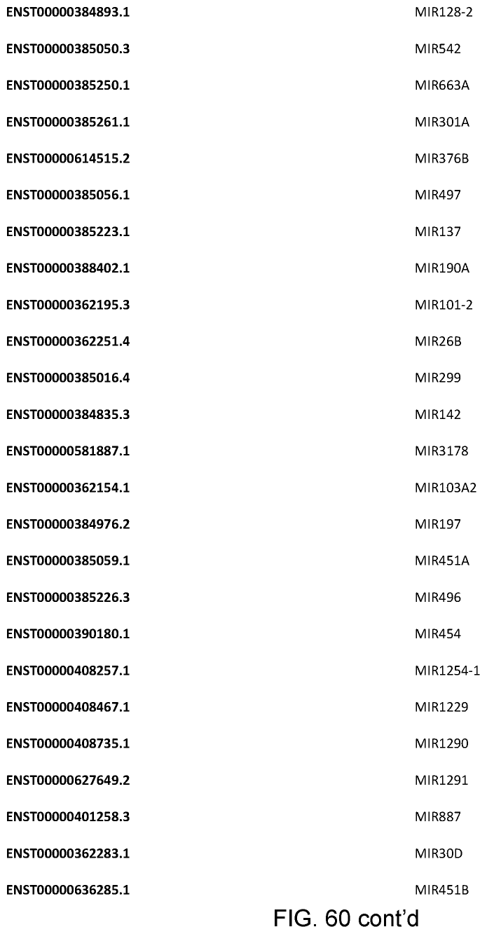

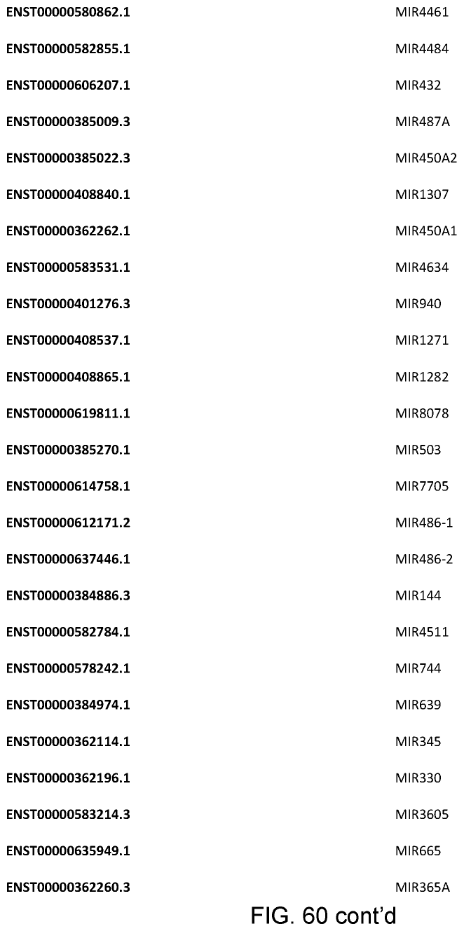

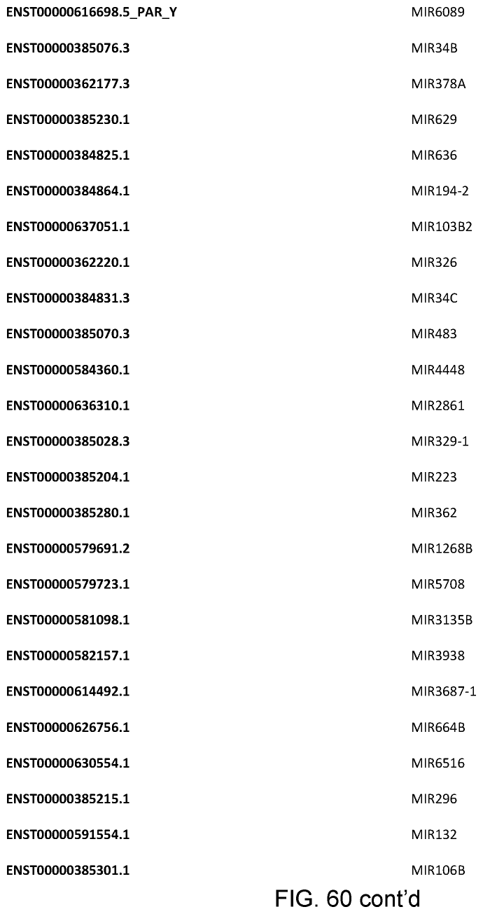

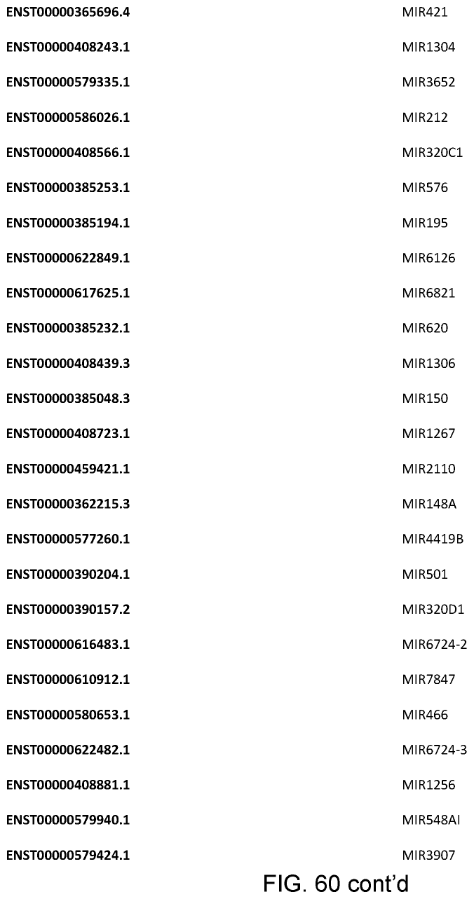

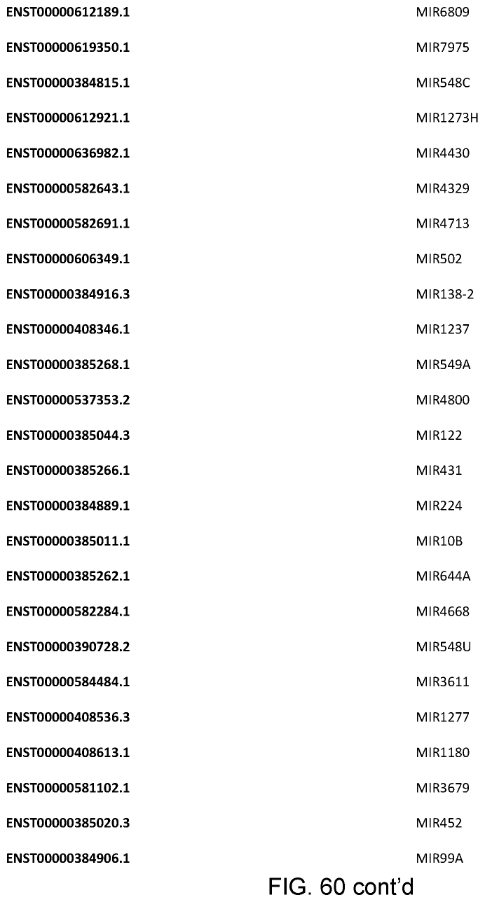

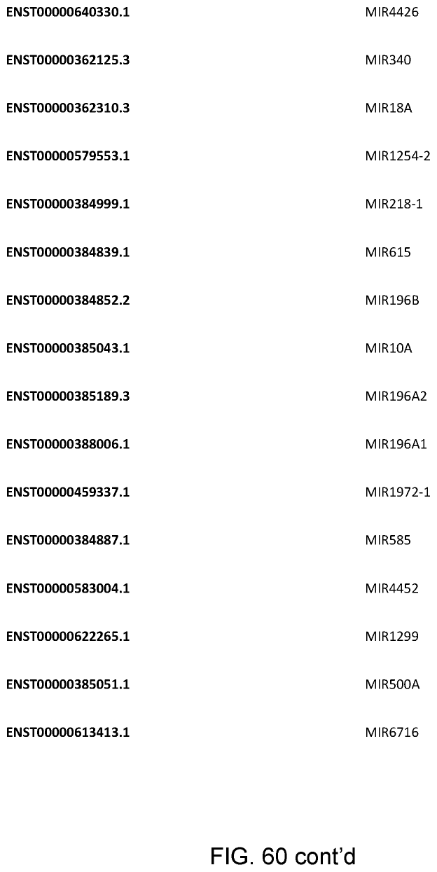

In some embodiments, the engineered exosomes comprise an osteoinductive factor and integrin α5 present at a higher amount than the amount present in a naturally occurring cell-derived exosome. In some embodiments, the at least one factors comprises one or more of let 7a, miR 218, miR 9-5p, miR 19a-3p, mir 30a-5p, miR 212-5p, miR 323-5p, miR 15a, miR 15b, miR 16, miR 424, miR 497, miR 424-, or integrin α5. In some embodiments, the at least one factor comprises one or more microRNAs listed in FIG. 60.

In some embodiments, the amount of the at least one factor in the exosomes is at least about 1.5-fold higher, about 3-fold higher, about 10-fold higher, about 11-fold higher, about 20-fold higher, about 50-fold higher, about 100-fold higher, about 115-fold higher, or about 200-fold higher than the amount present in the naturally occurring cell-derived exosome.

The number of engineered exosomes in an exosome composition can be any suitable number in order to provide or maintain a sufficient therapeutic or prophylactic effect. For example, in certain embodiments, the number of engineered exosomes in a composition is in a range of about 1×10² to about 1×10²⁰; for example, in a range of about 1×10² to about 1×10¹⁶, about 1×10² to about 1×10¹², about 1×10² to about 1×10¹⁰, about 1×10² to about 1×10⁶, about 1×10⁶ to about 1×10²⁰, about 1×10⁶ to about 1×10¹², about 1×10⁶ to about 1×10¹⁰, about 1×10¹⁰ to about 1×10²⁰, about 1×10¹² to about 1×10²⁰, or about 1×10¹⁶ to about 1×10²⁰. In certain embodiments, the number of engineered exosomes in the exosome composition is in a range of about 1×10⁶ to about 1×10¹².

Exosome compositions as described herein can be formulated into a composition suitable for administration in vivo. Thus, in one embodiment, exosome compositions of the disclosure, in addition to the isolated engineered exosomes as described herein, can further include a polymer carrier (e.g., a biodegradable polymer carrier).

In certain embodiments, the carrier includes one or more biocompatible polymers or oligomers. Examples of biocompatible polymers or oligomers include, but are not limited to, alginate, agarose, hyaluronic acid/hyaluronan, polyethylene glycol, poly(lactic acid), poly(vinyl alcohol), polyanhydrides, poly(glycolic acid), collagen, gelatin, heparin, glycosaminoglycans, saccharides (e.g., glucose, galactose, fructose, lactose, and sucrose), and self-assembling peptides. In certain embodiments, the biocompatible polymer is alginate, hyaluronic acid/hyaluronan, polyethylene glycol, poly(lactic acid), or poly(vinyl alcohol). In certain embodiments, the biocompatible polymer is alginate.

Particularly useful carriers suitable for the compositions of the disclosure are hydrogels. Thus, in some embodiments, the compositions comprise a hydrogel as the carrier.

A hydrogel of the disclosure, in certain embodiments, includes a plurality of biocompatible polymers or oligomers as described herein cross-linked with a hydrolysable linker. The linker can comprise an acrylate or a methacrylate, and optionally an ester, amide, or a combination thereof. In certain exemplary embodiments, the carrier is a hydrogel comprising alginate, hyaluronic acid/hyaluronan, polyethylene glycol, poly(lactic acid) or poly(vinyl alcohol), cross-linked with an acrylate linker or a methacrylate linker, and optionally an ester linker, amide linker, or a combination thereof.

In certain embodiments, engineered exosomes are bound to the carrier. To improve binding of the engineered exosomes with the carrier, one approach is for the carrier to mimic the cell adhesion capacity of native extracellular matrix (ECM) components. One approach includes incorporating a cell surface-binding factor into the carrier. Thus, in certain embodiments, one or more of the biocompatible polymers or oligomers of the carrier include a cell surface-binding factor. Such cell surface-binding factor can be a component of extracellular matrix, and is generally well known in the art. For example, in certain embodiments, the cell surface binding factor includes a fibronectin-derived peptide, a type I collagen-derived peptide, a peptide containing an MMP, or a combination thereof. The fibronectin-derived peptide is, for example, RGD. The collagen-derived peptide, for example, is DGEA (SEQ ID NO: 1) or GFPGER (SEQ ID NO: 2). For example, in certain embodiments, exosomes are bound to the cell surface binding factor on the carrier.

Carriers of the disclosure can also comprise a domain cleavable by one intracellular or extracellular release agent. In certain embodiments, carriers of the disclosure also comprise an enzymatic cleavable domain (e.g., a domain cleavable by one or more peptidases, proteases, esterases, elastases, etc.). In certain embodiments, carriers of the disclosure as otherwise described herein are cleavable by an intracellular or extracellular release agent. In certain embodiments, carriers of the disclosure as otherwise described herein are cleavable by two or more intracellular or extracellular release agents (e.g., wherein the carrier comprises two or more different chemical groups each cleavable by a different release agent). In some embodiments, the carrier comprises IPVSLRSGAGPEG (SEQ ID NO: 3), GPLGLAGGERDG (SEQ ID NO:4), GFLG (SEQ ID NO:5), GPMGIAGQ (SEQ ID NO:6), Phe-Leu, Val-Ala, Val-Cit, Val-Lys, Val-Arg, or Phe-Lys. In certain embodiments, the carrier comprises both the cell surface binding factor and the cleavable domain. For example, in certain embodiments, the carrier comprises GGGGIPVSLRSGAGPEG_DGEAY (SEQ ID NO:7).

Carriers of the disclosure can be present in an amount of 1% to 20% by weight based on the total weight of the composition. For example, in certain embodiments, the carrier is present in the amount of 1 wt % to 15 wt %, 1 wt % to 10 wt %, 1 wt % to 5 wt %, 5 wt % to 20 wt %, 5 wt % to 15 wt %, 5 wt % to 10 wt %, 10 wt % to 20 wt %, 10 wt % to 15 wt %, or 15 wt % to 20 wt %, based on the total weight of the composition.

In certain exemplary embodiments, compositions as described herein comprise 1×10⁶ to about 1×10¹² of the engineered exosome and the carrier present in the amount of 1 wt % to 15 wt %, based on the total weight of the composition.

The carrier can be provided in any form suitable for in vivo administration. For example, the carrier, such as the hydrogel, can be formulated in a variety of physical forms, including slabs, microparticles, nanoparticles, coatings, and films. In some embodiments, the hydrogel carrier of the present composition is formed by 3-D printing. In 3D printing, material is joined or solidified under computer control to create a three-dimensional object with material being added together (such as liquid molecules or powder grains being fused together), typically layer by layer. The most-commonly used 3D-printing process is a material extrusion technique called fused deposition modeling (FDM). The 3D-printing process builds a three-dimensional object from a computer-aided design (CAD) model, usually by successively adding material layer by layer.

Another aspect of the disclosure provides methods of preparing the compositions of the disclosure. Such methods include engineering stem cells to contain at least one factor that is: an osteoinductive factor, a neuronal regeneration factor, an immunomodulatory factor, and an extracellular matrix binding factor at a higher level than stem cells that are not engineered; and isolating the exosome from the cells. Any method of isolating exosomes from parental cells known in the art can be used to isolate exosomes as provided by the invention. In some embodiments, the engineering comprises genetic modification of the stem cells and/or and exposure of stem cells to a stimulus. In some embodiments, the genetic modification of the stem cells comprises overexpression of BMP2 and/or RUNX2. In some embodiments, the genetic modification of the stem cells comprises overexpression of one or more of the following factors: let 7a, miR 218, miR 9-5p, miR 19a-3p, mir 30a-5p, miR 212-5p, miR 323-5p, miR 15a, miR 15b, miR 16, miR 424, miR 497, miR 424, and integrin α5. In some embodiments, the genetic modification of the stem cells comprises overexpression of at least one of BMP2, RUNX2, OSX, LXRalpha, STAT6 and/or P13/Akt pathway members.

In some embodiments, the genetic modification of the stem cells comprises overexpression in an exosome-specific manner. In some embodiments, the exposure of stem cells to stimuli comprises culturing cells in the presence of one or more of ascorbic acid, β-glycerophosphate, and dexamethasone. In some embodiments, the exposure of stem cells to stimuli comprises treating cells with TNFα. In some embodiments, the exposure of stem cells to stimuli comprises exposing the stem cells to hypoxic conditions.

In some embodiments, the stem cells are mesenchymal stem cells. In some embodiments, the stem cells are dental pulp stem cells. In some embodiments, the method further comprises lyophilizing the isolated exosome to obtain a lyophilized isolated exosome.

### Definitions

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other component, feature, element, or step or group of component, feature, element, or steps.

As used in the specification and the appended claims, the singular forms “a,” “an” and “the” include plural referents unless the context clearly dictates otherwise.

As used herein, ranges and amounts can be expressed as “about” a particular value or range. About also includes the exact amount. Hence “about 5%” means “about 5%” and also “5%.” The term “about” can also refer to ±10% of a given value or range of values. Hence, about 5% also means 4.5%-5.5%, for example.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

As used herein, the term “engineered” relative to naturally occurring cell-derived vesicles, refers to cell-derived vesicles (e.g., such as exosomes, liposomes and/or microvesicles) that have been altered such that they differ from a naturally occurring cell-derived vesicles.

As used herein, the term “genetic modification” refers to the genetic manipulation of one or more cells, whereby the genome of the one or more cells has been augmented by at least one DNA sequence. Candidate DNA sequences include but are not limited to genes that are not naturally present, DNA sequences that are not normally transcribed into RNA or translated into a protein (“expressed”), and other genes or DNA sequences which one desires to introduce into the one or more cells, including promoter sequences that drive high levels of expression (i.e. cause overexpression). It will be appreciated that typically the genome of genetically modified cells described herein is augmented through stable introduction of one or more recombinant genes. Generally, introduced DNA is not originally resident in the genetically modified cell that is the recipient of the DNA, but it is within the scope of this disclosure to isolate a DNA segment from a given genetically modified cell, and to subsequently introduce one or more additional copies of that DNA into the same genetically modified cell, e.g., to enhance production of the product of a gene or alter the expression pattern of a gene.

### EXAMPLES

The Examples that follow are illustrative of specific embodiments of the disclosure, and various uses thereof. They are set forth for explanatory purposes only and should not be construed as limiting the scope of the disclosure in any way.

### Example 1: Osteoinductive Exosomes with Enhanced Binding to the Extracellular Matrix

Exosomes influence the fate of target cells: Depending on the source and target cell type, exosomes are endocytosed by either clathrin or caveolin mediated endocytosis. This process triggers endocytosis mediated signaling cascades in target cells mediated by the extracellular receptor kinase family (ERK) and mitogen activated protein kinase family (MAPK). The endocytosis of exosomes also results in the transference of their miRNA and protein cargo intracellularly. After this discovery, there has been increased focus on applications in regenerative medicine as inducers of cell proliferation, angiogenesis and as immunomodulators for cancer therapy. The miRNA and protein composition of the exosome is unique to the parent cell type it is sourced from and can vary in content and activity depending on the state of the source cell.

The significance and translational relevance for FATE: The potential of exosomes as SCLD mediators for regenerative medicine applications is high. While isolation is straightforward, it is not practical to isolate autologous exosomes without donor-dependent risks and variability in composition and potency. Some recent studies have shown that it is possible to package protein and genetic material into exosomes for therapeutic applications and cellular delivery. However, the inherent exosomal property to affect the recipient cell is often overlooked in favor of target delivery. With the recent knowledge on source and cell-type specificity of exosomes, the targeting of exosomes and the ability to engineer exosome functionality to induce SCLD are translationally relevant areas of investigation.

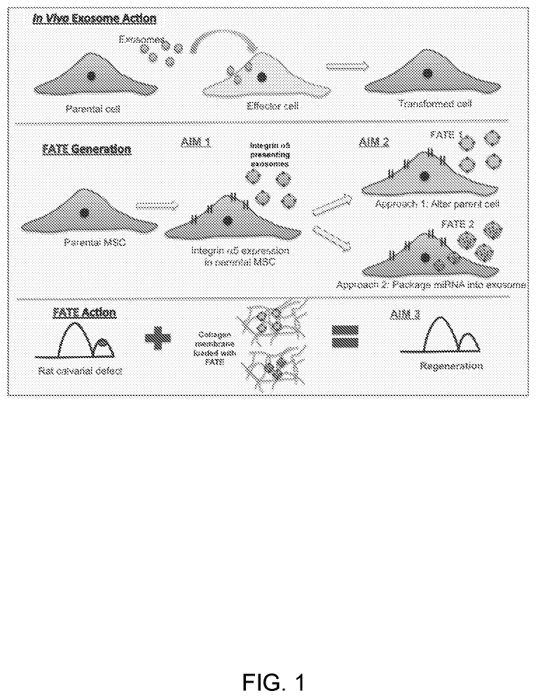

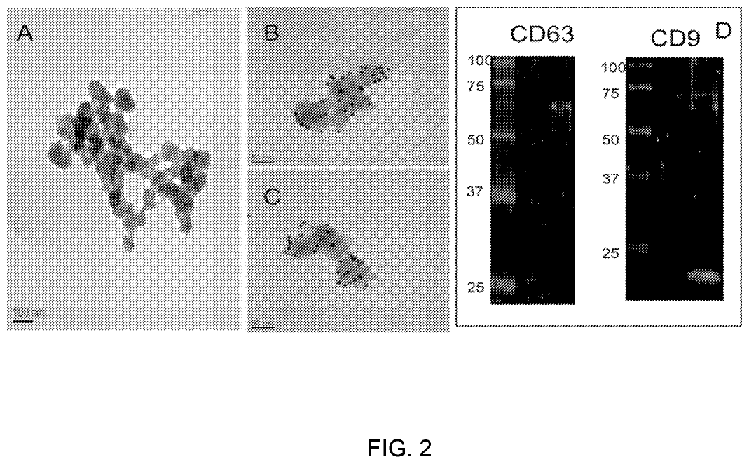

Primarily, targeting of exosomes can be engineered to be cell-type specific or biomaterial specific. Given the complexities of endocytic mechanisms of different exosomes by recipient cells, achieving cell-type specificity is not feasible. On the other hand, it is possible to achieve site-specificity or biomaterial specificity by controlling exosome-ECM interactions (FIG. 1). Biomaterials for tissue-engineering applications commonly contain ECM sequences. As exosomal membranes are subsets of the plasma membrane, specificity to these ECM proteins can be accomplished by modulating the expression of integrin α5 on the exosomal membranes. The translational significance of this approach is that it can be customized to impart specificity to any ECM component or motif by targeting appropriate transmembrane proteins/receptors.

Secondarily, by choosing application-specific cells as exosome sources (MSCs here), exosomes with specific functionality (FATE) can be engineered (FIG. 1). As FATE exosomes are engineered nano vesicles, they possess consistent properties without donor-dependent risks. Of additional translational significance are: 1) FATE can potentially be mass-produced using standardized cell lines. 2) Unlike the single morphogen system, FATE contain the necessary ‘information’ in the form of proteins and genetic material in physiologically relevant amounts to direct SCLD.

As a result of these drawbacks, bone regeneration is one of the most widely researched fields in regenerative medicine. Given the clinical need and the well-characterized system, bone regeneration has been chosen as a model system to study FATE. As many of the current allograft matrices for bone regeneration contain COL1 and FN (collagen membranes, DBM, etc.), the results of these experiments are translationally relevant to this field and to regenerative medicine in general.

Isolation of exosomes. Exosomes were isolated and characterized as per published protocols and as per standards developed for exosomal characterization. Exosomes were isolated from the culture medium of human marrow-derived MSCs (HMSCs). One day prior to isolation, the cell cultures were washed in serum free media and cultured for 24 hours in serum free media. The exosomes from the culture medium were isolated using the ExoQuick-TC (System Biosciences) exosome isolation reagent as per the manufacturer's protocol. The isolated exosome suspension underwent washing and buffer exchanges during the isolation procedure and was devoid of any measurable media constituents when purified. Exosome suspensions were normalized to cell number from the tissue culture plate they were isolated from and diluted to ensure that 100 μL of suspension contains exosomes isolated from 1 million cells as per the published and standardized protocols. Cross-verification will be performed by measuring RNA and total protein isolated from the exosome suspensions to ensure that RNA/protein concentration from the same volume of exosomes remained consistent. The presence of exosomes in the isolates was verified by transmission electron microscopy (TEM) (FIGS. 2A, B and C). For each batch of isolates, immunoblotting is also performed with exosome markers CD63 (Abcam, 1/1000) and CD9 (Abcam 1/1000) antibodies as positive markers (FIG. 2D) and also with tubulin as negative marker for intracellular proteins (Sigma 1/10,000).

Human bone marrow derived MSCs (HMSCs): All of the in vitro experiments to isolate exosomes and to test the potency of the exosomes are performed using HMSCs. The HMSCs that are routinely used are purchased from ATCC. These cells are primary human cells from healthy adult donors that have been certified and designated for research use. Each batch of cells obtained will be tested for multipotency to differentiate into osteogenic, chondrogenic and adipogenic lineages as per previously published protocols. The cells are not used beyond passage 4 for any application.

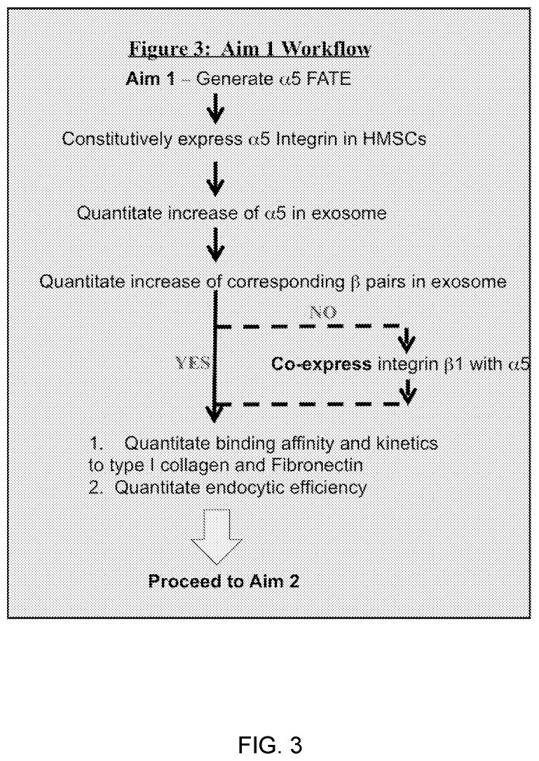

The preliminary results indicated that integrins meditate binding of exosomes to ECM proteins. However, targeting of FATE for regenerative medicine requires improved binding efficiency to biomaterials. As the exosomal membrane is a subset of the plasma membrane, improving the biomaterial-ECM binding characteristics (affinity/rate or both) of exosomes was attempted by increasing the expression of integrin α 5 on the exosomal membrane (FIG. 3). Integrin α 5 and its respective β pairs mediate cellular adhesion to ECM proteins FN, COL1 and to the RGD sequence. Furthermore, it was established that increased integrin α 5 expression results in a concurrent enhancement in ECM mediated adhesion.

COL1 is the most abundant ECM protein and forms the primary constituent of the organic bone matrix upon which hydroxyapatite is nucleated. Therefore, several biomaterials that are used clinically (Collagen sponges and DBM) as well as experimental materials (blends of collagen and other polymeric biomaterials used to alter material properties) contain COL1 as the primary constituent. The second most abundant ECM protein is FN. The RGD domain was in fact originally identified in domain 10 of the FN protein sequence. Several clinical materials such as DBM and allograft bone particles contain this structural matrix protein. Therefore, a significant amount of biomaterials developed for regenerative applications also contain integrin-binding domains from FN.

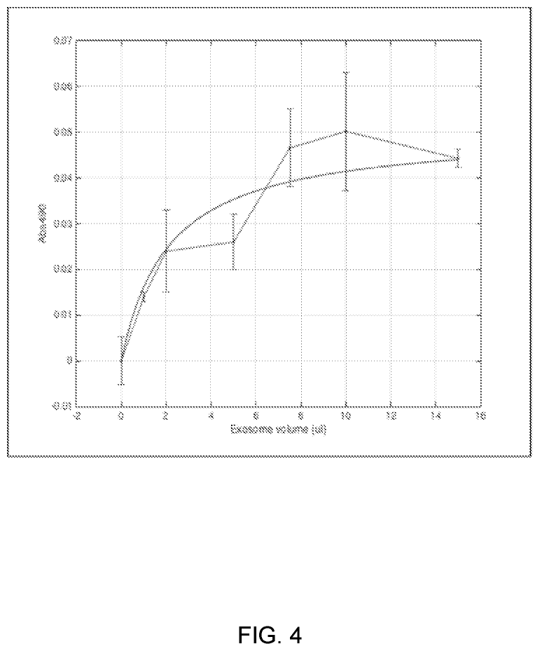

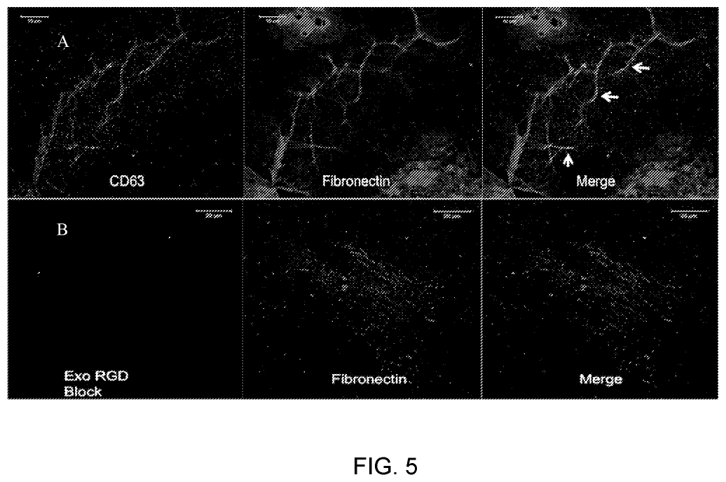

Exosome binding to COL1 and FN: The preliminary results indicate that exosomes can bind dose dependently and in a saturable manner to COL1 (FIG. 4). Exosomes can also bind to FN secreted by MSCs and this binding is integrin mediated (FIG. 5A). Blocking integrin binding using the RGD peptide (25 mM) abrogated exosome binding to FN and to the ECM of MSCs (FIG. 5B).

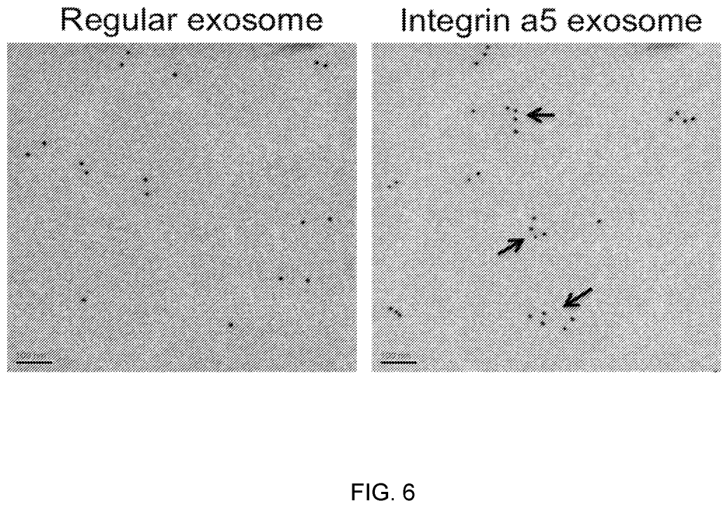

Engineering FATE displaying increased α 5 integrin (α 5 FATE): As the exosomal membrane is a subset of the plasma membrane of the parent cell, an increase in integrin α 5 expression on the plasma membrane of the parent cell consequently results in the increased presence of integrin α 5 on the exosomal membranes. HMSCs are transduced to constitutively express integrin α 5. Plasmids containing the integrin α 5 gene under the control of the EF1 α promoter (suitable for expression in primary MSCs) are readily available (Applied biological materials (ABM), Canada). Following transduction, puromycin selection is employed to generate a stable cell line that constitutively expresses integrin α 5 as per previously published protocols for transduction and cell-line generation. Exosomes are isolated from this cell line as per previously described protocols. Preliminary results indicate that exosomes isolated from HMSCs constitutively expressing integrin α 5 show increased presence of the same on the exosomal membranes (FIG. 6).

Evaluation of increased integrin presence in α 5 FATE: Quantitative and qualitative evaluations will be performed on α5 FATE with respect to control exosomes. Preliminary results presented in FIG. 6 indicate an increase in the expression levels of integrin α 5 in the α 5 FATE compared to control exosomes. TEM analyses of different batches of purified exosomes immunogold labeled for integrin α5 will be used to qualitatively observe the increased integrin presence. Quantitative evaluations are performed by NanoSight analysis of immunolabeled exosomes. The NanoSight instrument is specifically designed to detect fluorescently labeled nano particles such as quantum dots and exosomes. Control and α5 FATE will be fluorescently dual labeled with CD63 (exosome marker) and integrin α 5 antibodies in different wavelengths. The fluorescence corresponding to both proteins are quantitated using the nano-sight instrument. The CD63 antibody fluorescence coupled with size exclusion nanosight analysis is used to count the number of exosomes in each pool followed by estimation of integrin α5 presence in the form of fluorescence intensity per exosome. A comparison between the control exosomes and α5 FATE is performed and expressed as percentage gain over control to quantitate the increase in the expression levels of integrin α5. In addition to evaluating the increase in α5 integrin expression, the increased presence of its corresponding β pairs is also evaluate using the same methodology described above. In particular, if there is an increase in the expression levels of β1, β3 and β5 integrins is also evaluated. These candidates were chosen based on published characterizations of integrin pairs binding to COL1 and FN.

Evaluation of exosome dose-dependent binding to COL1 and FN (constant time): Quantitative binding experiments are performed to evaluate binding of control exosomes and α5 FATE to COL1 and FN as per the previously published protocols. Briefly, 96 well assay plates coated with 5 μg of COL1 or FN are incubated with increasing amounts of fluorescently labeled exosomes for a period of 2 hours (0-20 μl, refer to FIG. 3) at room temperature. Green fluorescence labeling of exosomes is performed using the Exo-Glow labeling kit (System Biosciences) as per the previously published protocols. As stated previously, normalized suspensions (100 μL of suspension containing exosomes from 1 million cells) are used. The bound exosomes are quantified using a micro titer plate reader (BioTek). The total exosome amount (x-axis) is plotted against normalized fluorescence readings and the resulting plot is fit to a rectangular hyperbola (the standard for a single binding site saturation). FIG. 4 serves as an example. Any improvement in binding is observed as saturation at lower amounts of exosome with respect to controls. As the number of integrins on the exosomes cannot be presented as a concentration, calculation of a dissociation constant (K_{D}) is not possible. However, the change in affinity can be quantitated in the form of reduction in required amounts of exosome to achieve saturation.

To demonstrate that the improvement in binding is a direct result of α5 presence, a competitive binding experiment is performed using integrin α5 antibody. Briefly, α5 FATE is pre-incubated with integrin α5 antibodies to saturate all α5 integrins. The quantitative binding experiments to COL1 and FN are performed in conjunction with untreated controls to observe the percentage loss in binding. Further, the effect of RGD blocking on the binding efficiency will also be quantified. For these experiments, the collagen and FN amounts are kept constant at 5 μg. α5 FATE are maintained at saturation volume and is pre-treated with increasing concentrations of the RGD peptide (SIGMA). The corresponding dose-dependent reduction in binding efficiency to COL1 and FN is quantitatively analyzed using the binding assay.

All experiments are performed in quadruplicates. Statistical significance for all comparisons between control exosomes and α5 FATE or for significance of the competitive binding experiments is evaluated using student's t-test with a 95% confidence interval.

Estimation of binding kinetics (variable time): An increase in receptor presentation on the exosome membrane can increase the rate at which exosomes can bind to COL1 and FN. From a translational perspective, this is an important property to consider, as it would reduce the time of contact between an exosome suspension and a biomaterial to achieve binding saturation. A time course assay is used for this purpose. Pseudo first order kinetics was followed by maintaining COL1 and FN concentration at 5 μg/coated 96 well and control exosomes or α5 FATE are used 5× saturation amount of α5 FATE (a higher than saturation concentration is required to satisfy pseudo first order kinetics). Control exosomes and α5 FATE are used at the same amounts to compare improvement in kinetics. The fluorescently labeled exosome suspensions are incubated with the ECM proteins at room temperature in fixed time increments of 5 minutes up to 60 minutes. The amount of bound exosomes after each time point is quantitatively measured using a plate reader and plotted as fluorescence intensity versus time plot. The slope of the plot (dFluorecence/dT) is calculated to estimate the rate of binding. Statistics are performed as described above.

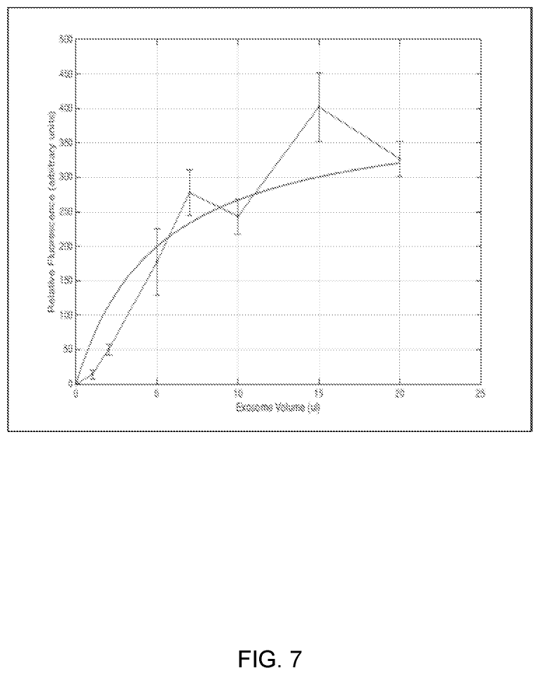

Endocytosis of α5 FATE: The ability of α5 FATE to be endocytosed by HMSCs is evaluated quantitatively in a dose dependent manner as per published protocols using fluorescently labeled exosomes. The preliminary results indicate that MSC derived exosomes are endocytosed in a dose-dependent and saturable manner by target HMSCs (FIG. 7). Therefore, the ability of α5 FATE to be endocytosed by HMSCs is determined and compared to that of control exosomes. A loss is efficiency is characterized as a statistically significant drop in the amount of endocytosed exosomes (quantitated as a measure of fluorescence intensity at each concentration) and/or a statistically significant increase in the amount of exosomes required to saturate endocytosis (an indicator of slow/impaired endocytosis). The experiments are performed at 37° C. with 1-hour incubations. A standardized exosome dosage of 0 to 20 μl is used. Each experiment will contain 6 repeats. The significance between the control group and α5 FATE is analyzed using student's t-test (95% confidence).

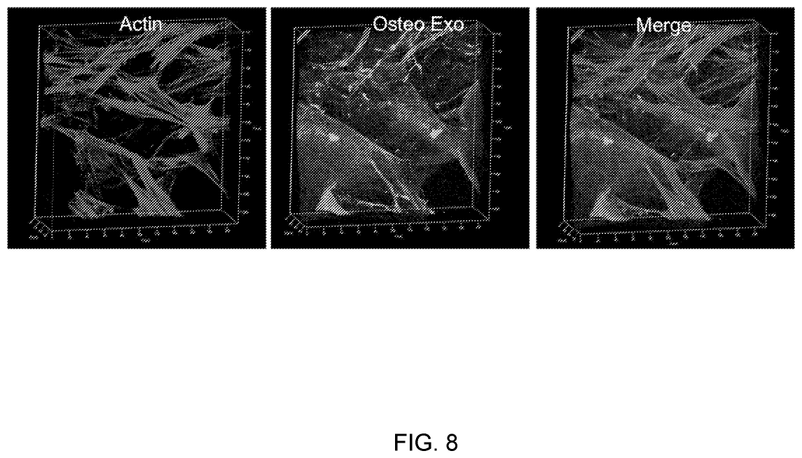

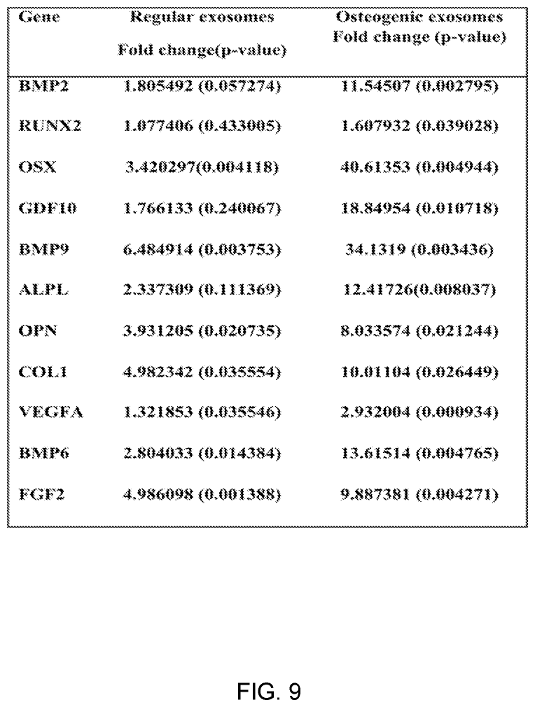

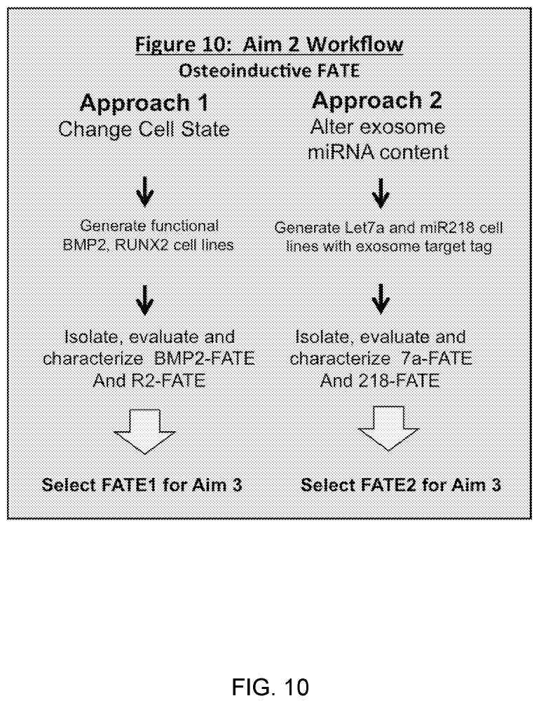

Endocytosis of α5 FATE bound to ECM proteins: MSC derived exosomes can be endocytosed by target MSCs when bound to COL1 membranes, (FIG. 8). These bound exosomes were also functional in in vivo experiments (refer to FIG. 9 and FIG. 10). The ability of α5 FATE, when bound to COL1 and FN coated plates, to be endocytosed by HMSCs is evaluated quantitatively and qualitatively. Fluorescently labeled exosomes (control and α5 FATE) is bound at increasing concentrations to COL1 and FN coated cover glass bottomed assay plates (5 μg/well). 25,000 HMSCs will then be seeded on to the plates and incubated for 24 hours in tissue culture conditions. For qualitative evaluations, the plates are imaged by confocal microscopy. For quantitative evaluation, the cells are trypsinized, fixed in neutral buffered formalin and subjected to FACS (fluorescence activated cell sorting) analysis to identify the percentage of cells that have endocytosed the labeled exosomes and also the intensity of the signal to correlate with dose dependency. Qualitative evaluations and verification of results in a 3D environment are performed by binding fluorescently labeled α5 FATE to COL1 membranes (Zimmer collagen membranes) followed by HMSC seeding (250,000 cells/1 cm square membrane for 24 hours). The formalin fixed scaffolds are subjected to z-stack confocal imaging as per the published protocols, (FIG. 8). All experiments are performed in quadruplicates. Statistical evaluations are performed using student's t-test (95% confidence interval).

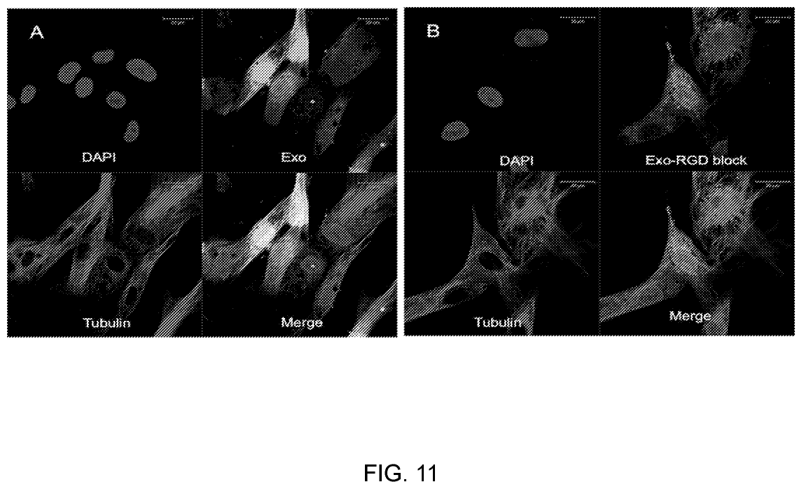

It was expected that α5 FATE will be endocytosed with the same efficiency of control exosomes irrespective of the increase in rate or stoichiometry of the binding. This is based on the fact that MSC exosome endocytosis is not integrin mediated (preliminary result provided in FIG. 11). Direct FATE mediated SCLD (osteoinduction) was evaluated. The workflow provided in FIG. 10 gives a broad overview of this evaluation.

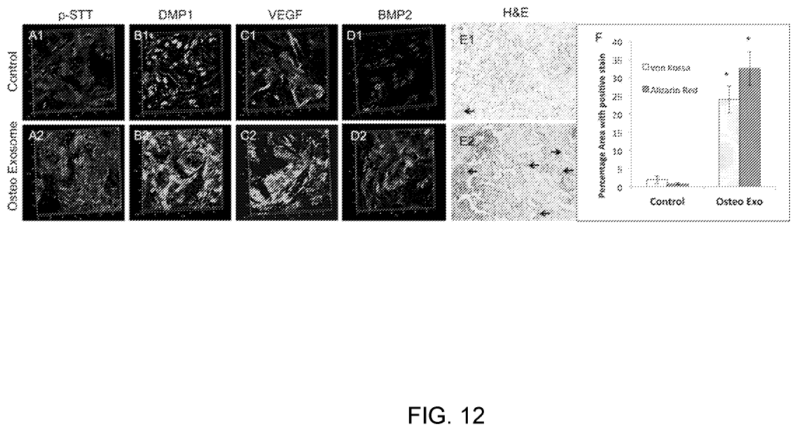

The preliminary results (FIG. 9, FIG. 12 indicated that exosomes from osteogenically differentiated MSCs are better inducers osteogenic SCLD compared to exosomes isolated from undifferentiated MSCs. However, it is not possible to generate exosomes of consistent functionality for therapeutic applications using this approach. Therefore, the osteogenic potential of MSCs is stably enhanced by constitutively expressing known osteoinductive morphogen BMP2 and the well-defined osteoinductive t transcription factor RUNX2, respectively.

Exosomes from differentiated MSCs are more potent inducers of osteogenic SCLD: Human bone marrow derived MSCs (HMSCs) were subjected to osteogenic differentiation for 4 weeks in the presence of osteogenic culture (containing ascorbic acid, β-glycerophosphate and dexamethasone). Exosomes were isolated from both the differentiated MSCs and MSCs cultured in regular media (control). Using the control or osteogenic exosomes as inducers of SCLD, undifferentiated HMSCs were subjected to 3D in vitro differentiation assay for 48 hours followed by qPCR analyses of osteoinductive gene expression. Compared to control exosomes, treatment of HMSCs with exosomes from osteogenic MSCs resulted in a significantly higher expression of a broad panel of osteogenesis-associated genes (FIG. 9). Notably, the experiment was performed in the absence of other differentiation factors to observe the effect of exosomes alone.

When exosomes from control and osteogenic MSCs were bound to collagen scaffolds and implanted in vivo (subcutaneously) with HMSCs, the osteogenic exosomes induced a more robust expression of phosphorylated proteins (required for induction of matrix mineralization and identified using an antibody directed to phosphorylated serine, threonine and tyrosine residues), mineralization inducers such as dentin matrix protein 1 (DMP1) the pro-vascular protein VEGF and osteoinductive growth factor BMP2 (FIG. 12). Concurrently, histological evaluations revealed increased vascularization (arrows in FIG. 12E2) and a significant increase in calcium phosphate deposition as depicted by quantitated alizarin red and von Kossa stains (FIG. 12F). These results show the osteoinductive potential of exosomes from differentiated MSCs.

Generation of osteoinductive FATE from BMP2 and RUNX2 expressing HMSCs: Transduction of BMP2 and RUNX2 genes individually in α5 HMSCs generated as provided above was performed. Plasmids encoding the BMP2 and RUNX2 gene suitable for MSCs are commercially available (Applied Biological Materials, Canada). Given that α5 HMSC has been selected using puromycin for stable expression of α5 integrin, the BMP2 and the RUNX2 expression vectors will contain a neomycin cassette for selection. The resultant BMP2 or RUNX2 gene (qPCR) and protein expression (western blotting, IF, ELISA) in the derived cells are evaluated quantitatively with respect to wild type and vector controls to confirm constitutive expression. A qPCR analysis of the expression of osteogenic marker genes is performed to evaluate the increased osteogenic potential of both the derived cell lines as a confirmation of the functionality of the constitutively expressing proteins. GAPDH and B2M are used as internal controls. The osteoinductive marker genes are: Growth factors: BMP2, BMP6, TGFβ1, VEGFA, FGF2, GDF1. Transcription factors: RUNX2, Osterix (OSX). ECM proteins: Osteocalcin, Alkaline phosphatase, COL1, osteopontin and DMP1. This list of genes is based on the published experience in bone and mineralized tissue biology. FIG. 9 is an example of a typical data set. Exosomes from the of α5-BMP2 and α5-RUNX2 HMSCs cell lines are isolated as BMP2-FATE and R2-FATE.

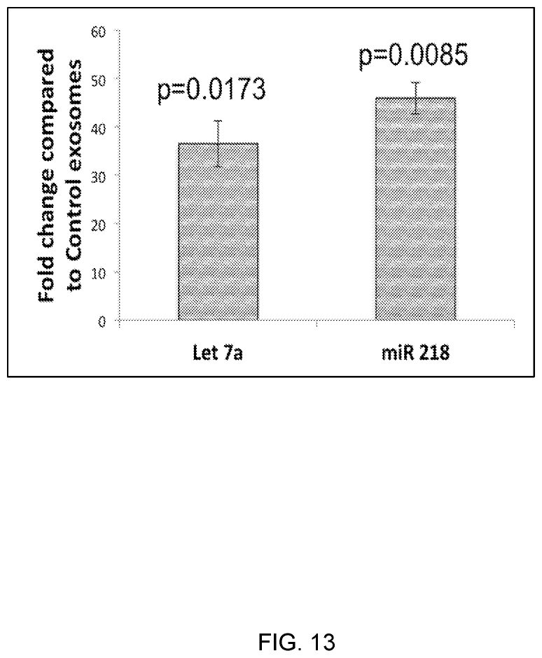

miRNAs play a pivotal role in exosomal function. Therefore, specific manipulation of exosomal miRNAs may be used to control exosome functionality. Two miRNAs (Let7a and miR218) that are present in increased amounts in exosomes from differentiated MSCs (FIG. 13) were identified. The Let-7 family of miRNAs has been shown to enhance osteogenic differentiation of MSCs. On the other hand, miR-218 enhances osteogenic differentiation of MSCs by positively regulating the Wnt/β-catenin signaling cascade. Therefore, they are good targets for manipulation.

Recent studies on miRNA sorting into exosomes have identified a target sequence in the 3′ end of miRNAs (GGAG; SEQ ID NO:8) that directs exosomal sorting. Plasmids and expression systems that readily express this target sequence at the 3′ end of miRNA sequences are commercially available (System Biosciences, XMIR expression system) and have been verified experimentally. Using this system, Let7a and miR218 into MSC exosomes are selectively packaged to generate osteoinductive FATE.

Generation of osteoinductive FATE by exosomal expression of Let7a and miR218: α5 HMSCs is transduced with plasmids incorporating the Let7a and miR218 sequences individually along with the exosomal targeting sequence and selected for stable expression as described previously. Exosomes are isolated from these cell lines as described and labeled as 7a-FATE and 218-FATE respectively. miRNA is isolated from these exosomes and qPCR are used to evaluate the expression levels of Let7a and miR218 in the respective exosomes with respect to control exosomes and vector-control exosomes (FIG. 13 data is an example). Statistically significant (t-test, P<0.05) increase in the expression of Let7a and miR218 in 7a-FATE and 218-FATE respectively with respect to the controls will denote success in the generation of FATE.

In vitro evaluation of osteoinductive potential: This evaluation is performed on FATE isolated using both approaches. A 3D in vitro cell culture system (COL1 scaffolds) is used to evaluate the osteoinductive potential of FATE. This 3D model provides a biomimetic environment for the MSCs providing an ideal environment to evaluate the osteoinductive potential of exosomes. For this experiment, the standardized exosome to cell ratio of exosomes from 500,000 cells per 100,000 HMSCs is used. This number was derived from exosome endocytosis saturation experiments. 500,000 HMSCs is seeded on to either control or exosome bound COL1 scaffolds (1 cm×1 cm Zimmer collagen tape). Exosome suspension is adsorbed on to the collagen tape and incubated at room temperature for 10 minutes prior to cell seeding. The cells are cultured within the scaffolds for 2, 4 and 7 days. The experiments are conducted in quadruplicates and HMSCs treated similarly in the absence of exosomes will serve as comparative standard for gene expression data. Exosomes from undifferentiated HMSCs will serve as control for exosome basal activity. Osteogenic exosomes from differentiated HMSCs that have osteoinductive properties are used as positive control. Four different osteoinductive FATE form the experimental groups.

RNA is isolated from the control and experimental groups at different time points. The expression levels of genes required for and indicative of induction of osteogenic differentiation (list of genes provided above, FIG. 9) are evaluated by qRT PCR with respect to controls as per standard protocols. Statistical significance is assessed using student's t-test with respect to the control (non-exosome containing) group with 95% confidence interval. ANOVA is used to analyze the significance when multiple groups are compared as well as for comparing the different osteoinductive FATE.

Evaluation of ECM binding and endocytic potential: The methods to generate FATE should not affect their ECM binding or endocytic potential of the exosomes. However, their ability to bind to COL1 and FN as well as their ability to be endocytosed by HMSCs is verified by performing the quantitative binding and endocytic assays as described above.

FATE—directed tissue regeneration (bone repair) in vivo was evaluated. The acid test for any osteoinductive strategy is the ability to induce repair of critical size bone defects. Bone regeneration using osteoinductive exosomes delivered in clinically relevant biomaterials is an ideal model to test the translational relevance of FATE in regenerative medicine. Therefore, the two types of FATE using the well-developed and standardized rat calvaries defect model are evaluated. In order to maintain clinical relevance, a clinical grade collagen membrane (Zimmer collagen tape) is used as the carrier for FATE and control exosomes.

Loss of function-ECM binding: The rationale behind including this group is to show the importance of FATE targeting in bone repair and tissue regeneration. Disruption of ECM binding is achieved by pretreating FATE with 2 mM RGD peptide (FIG. 5 provides relevant data for choice of concentration). FATE is expected to show no/impaired binding to the collagen membranes resulting in impaired/reduced osteoinduction due to lack of localization to the defect site. The RGD-treated FATE is bound to the collagen membranes and treated the same way the other groups.

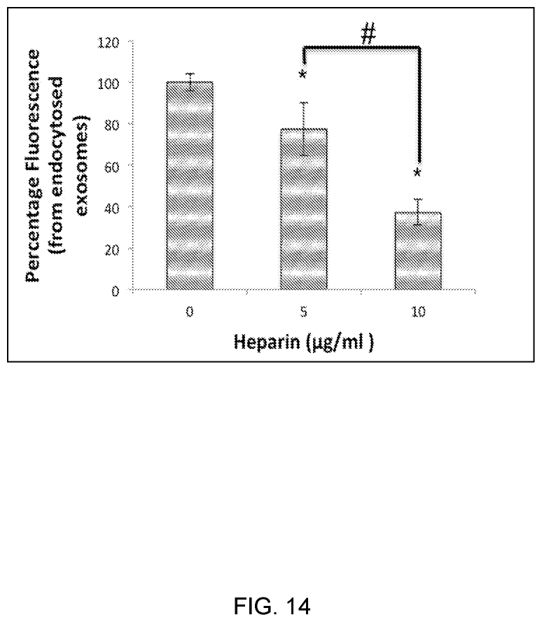

Loss of function-Endocytosis: Endocytosis of exosomes is a critical process that delivers the osteoinductive molecules enclosed within the exosomal membrane into target cells. To highlight the functional importance of FATE in bone repair, exosome endocytosis is blocked by using sulfated heparin (Sigma). To achieve this, the MSCs are pretreated with 10 μg/ml heparin (FIG. 14). Additionally, collagen membranes that are used to bind FATE are pre-treated with 50 μg of heparin. Heparin binding to COL1 is well characterized. Therefore it is possible to load the collagen membranes with heparin. The preliminary results (FIG. 14) indicate that MSC exosomes are endocytosed via cell-surface Heparin Sulfate Proteoglycan Receptors (HSPGs). Sulfated heparin can bind to these HSPGs and block MSC exosome endocytosis. The results presented in FIG. 14 indicate that there is a dose-dependent reduction in the endocytosis of fluorescently labeled MSC exosomes in the presence of heparin. 5× saturation concentrations are used for the animal experiments to ensure that all cells within the defect boundary receive heparin treatment.

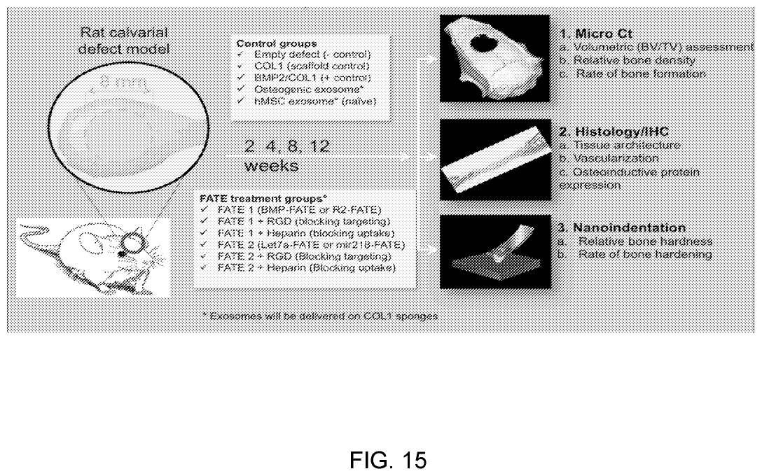

Rat calvarial defect model: All surgeries are performed as per approved animal care protocols. A critical size calvarial bone defect 8 mm in diameter would be made using a trephine bur without dura perforation as per established standards. For the groups containing exosomes or FATE along with the collagen membrane, the exosome/FATE suspension (100 μl, equivalent of exosomes from 1 million cells) is added to the biomaterial just before surgery and incubated for 10 minutes at room temperature to facilitate binding. Two different FATE (FATE1, FATE2, one from each approach in aim 2) are used. Naïve MSC exosomes are used as a control group and osteogenic exosomes are used as a positive control. FIG. 15 shows all the control and experimental groups. The animals are sacrificed 2, 4, 8 and 12 weeks post-surgery. The time points have been chosen from as early as 2 weeks to observe the rate of formation of mineralized matrix between the groups. There are 6 experimental repeats (n=6) per group per time point (based on power analysis: 80% power, 95% confidence). Three of them are male and three are female rats to ensure absence of gender bias in the results. For all in vivo experiments, wild type rats are used. After euthanasia, the calvarial bones are fixed in neutral formalin and processed for:

Quantitative μCT: For these experiments, extracted bone blocks are fixed in formalin and scanned using a μCT-40 scanner (Scanco Medical, Wayne, Pa., USA). Scan parameters are 90 KVp (voltage), 5 mA (tube current) and an integration time of 1 min. Reconstruction of the 2D slices into 3D images is performed using the manufacturer's software. μCT is used to analyze the following:

1. Volume of bone regenerated: The volume of regenerated bone at the various time points are quantified with respect to the total void volume. Statistical significance (P<0.05) is calculated using ANOVA for multiple group comparisons and pair wise comparisons are using Tukey's method. This type of evaluation will provide quantitative data on the rate of bone repair (slope of volume vs time plot) amongst the groups.

2. Quality of regenerated bone: Quality of regenerated bone (bone density) is obtained by quantitating the average radio opacity of the regenerated area with respect to that of the surrounding natural bone. Statistical analyses amongst groups are performed as described above. The radiopoacity is an indirect measure of bone density. Data from various groups and time points will provide a quantitative analysis of the rate of bone hardening (slope of radio opacity vs time plot) as well as the quality of the FATE regenerated bone in comparison to the control groups and natural bone.

Nano indentation: Nano indentation experiments are performed to analyze the actual hardness of the FATE regenerated bone with respect to that of native bone and the control groups. In addition, the evaluation of bone hardness from the various time points will also provide quantitative information on the rate of bone hardening amongst the groups (slope of hardness vs time plot). Nano indentation measurements are performed as per the previously published protocols. All measurements are performed at room temperature using a calibrated TI-700 Ubi nanoindentation system (Hysitron, Inc.). A 100 μm cono-spherical tip is used for a trapezoidal load pattern. 12 indents are made per sample randomly spanning the defect area and 12 across normal bone. The modulus is calculated using the Oliver Pharr method and the hardness is calculated using the formula: H=Pmax/Ar, where Pmax=maximum load and Ar=residual indentation area. The data is represented as hardness in GPa and is compared to hardness of surrounding normal bone at all time points. Statistical significance between the groups (P<0.05) is calculated using ANOVA and pair wise comparisons is evaluated using Tukey's method.

c) Histology: The samples are decalcified prior to histology. The samples are embedded in paraffin and sectioned along the x-z plane into 5 μm thick sections to view the injury closure across the thickness of the bone, as per the previously published protocol. Two sections from the top, middle and end (along y direction) of each block is analyzed using:

H&E stain: This is used to qualitatively analyze tissue architecture, osteoblast/mesenchymal cell infiltration and presence of blood vessels. Semi quantitative analyses on MSC infiltration and percentage vascularization is performed as per the previously published methods.

IHC for osteogenic marker proteins: Fluorescence IHC is performed to analyze qualitatively, the expression patterns and levels of marker proteins osteocalcin (OCN), Bone sialoprotein (BSP) and dentin matrix protein 1 (DMP1) between groups. The sections are probed to analyze the expression pattern of BMP2, TGFβ and VEGF among the groups and compare it to native bone.

Overall, the disclosed studies enable the generation, characterization and evaluation of FATE as nano-scale mediators of SCLD for bone regeneration.

### Example 2: Immune-Modulating Osteoinductive Exosomes

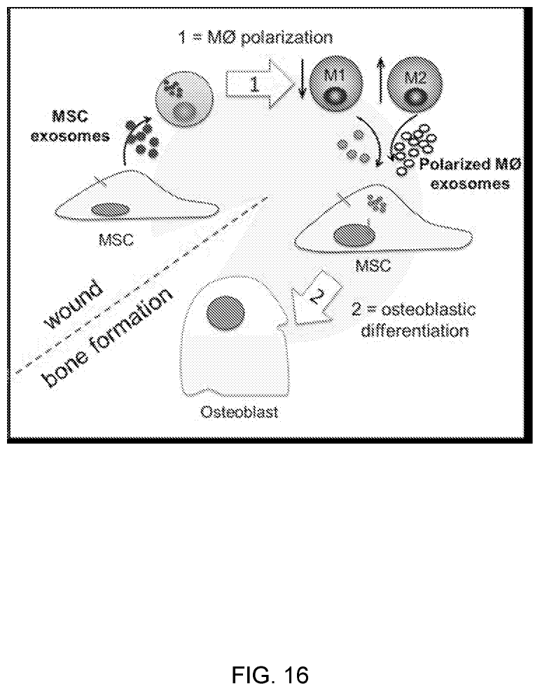

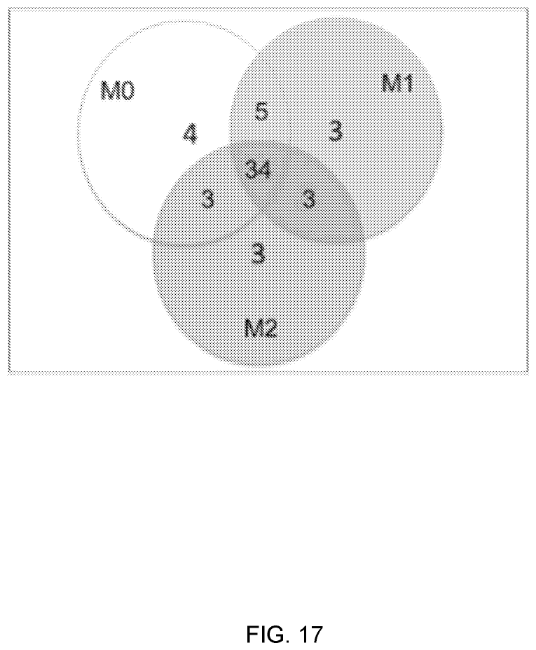

The mesenchymal stem cell's (MSC's) osteoinductive and immunomodulatory signaling is well known and involves macrophages (MØ). The studies indicate MSCs and their exosomes function by negatively regulating M1 polarization that reduces the M1/M2 MØ ratio in healing bone tissues and M2 MØ exosomes stimulate osteogenesis and bone regeneration. Taken together, these results indicate the presence of an immunomodulatory loop involving MSCs and MØ polarization to promote repair and regeneration (FIG. 16).

Exosomes (nanosized vesicles enriched in miRNAs) are significant components of secretome signaling among cells. MSC exosomes are implicated in the control of bone repair via MØ. Although several lines of evidence exist for the immunomodulatory properties of MSCs, there is a significant gap in knowledge regarding the immunomodulatory roles of both MSCs and MØ exosomes. Studying these mechanisms provides valuable information that can be used to engineer immunomodulatory and regenerative exosomes for therapeutic use. It is hypothesized that MSC exosomes regulate MØ polarization resulting in MØ exosomes that contribute to the control of bone repair by reducing the M1/M2 MØ ratio in healing tissues to foster M2 MØ osteoinductive signaling (FIG. 16).

MSCs influence MØ polarization in health and disease. It is hypothesized that inflammation-informed MSC exosomes and their miRNA cargo direct the signaling of MØ polarization (e.g., M1/M2 ratio) during bone regeneration. The effect of naïve and inflammation-informed MSC exosomes and miRNA cargo on MØ polarization is determined by immunocytochemistry in vitro, How MSC exosomes and specific miRNAs affect MØ polarization signaling pathways (e.g., M1=NF [symbol] B, Notch, SOCS3; M2=AKT, Stat6, LXRα) are characterized in cell culture studies. The impact of naïve and inflammation-informed MSC exosomes on the temporal changes in M1 and M2 MØ populations within healing calvaria defects is defined in vivo. The identified MSC exosome miRNAs are demonstrated to contribute to the MSC's immunomodulatory properties (e.g. ↓M1/M2 ratio) to enhance bone repair.

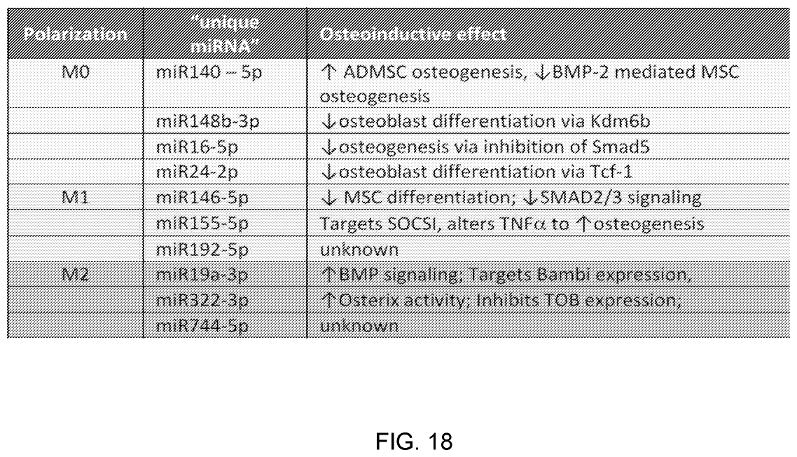

Further, MØ exosome cargo varies with polarization to directly influence healing. The preliminary work has identified polarity-specific miRNAs associated with osteoinduction in MØ exosomes (FIG. 17 and FIG. 18). It is hypothesized that polarity-specific miRNA in primary MØ exosomes influence MSC osteoblastic differentiation and bone regeneration. Based on the promotion of M2 MØ (↓M1/M2 ratio) in healing calvaria, it is a) affirmed by miRNAseq followed by qPCR that polarized M2 MØ exosomes contain osteoinductive miRNAs, b) defined M2 MØ exosomal miRNAs' osteoinductive mechanism(s) by i) in silico miRNA target analysis, ii) defining effects of overexpression and knock down of selected miRNAs on targeted gene/protein expression and osteoblast differentiation; and c) studied in the mouse calvaria model the impact of M2 MØ related exosomes and miRNAs on bone regeneration. Existing anti-inflammatory approaches impact M1 polarization and here M2 exosome mechanisms that directly promote osteoinduction in bone repair are examined. These mechanistic studies explore the roles of MSC and MØ exosomes in an immunomodulatory loop that influences regeneration. Additionally, the potential to manipulate this exosome signaling mechanism to enhance immunomodulation and bone repair is demonstrated.

MØ-induced osteogenesis has been interrogated in cell culture; a MØ polarity-dependent expression of MØ osteoinductive cytokines was previously identified. Other macrophage-derived mediators of osteoinduction have also been identified, including OSM, SDF-1, PGE-2 and TGF-β. The role of MØ in osteoblast physiology has been informed by cell culture studies demonstrating that: a) MØ-derived cytokines promote osteoblastic differentiation, b) osteoblast/MØ and MSC/MØ co-culture promote osteoinduction and c) depletion of MØ from bone marrow reduces CFU-OB formation. Cell culture studies also demonstrated d) that biomaterial/MØ interactions influence the MØ osteoinductive function. e) In vivo, different approaches to MØ depletion (e.g., systemic monocyte or MØ depletion, clodronate, MaFIA mice) result in reduced fracture healing and bone repair. These studies implicate the MØ, but have not revealed the mechanisms acting in the required communication between MSC and MØ (and potentially other cell types).

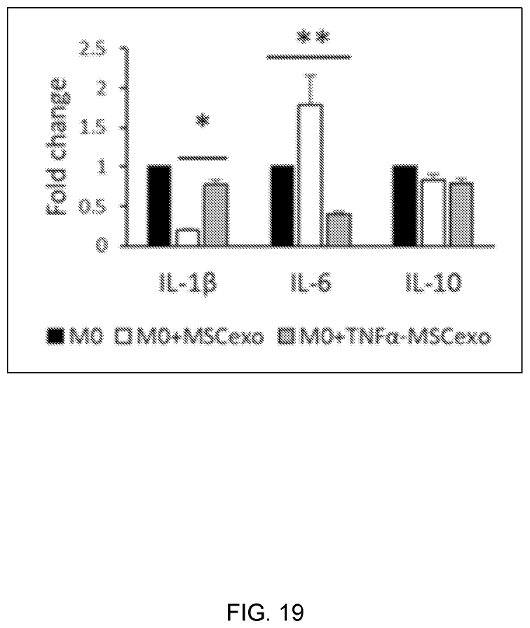

The regenerative function of MØ involves the regulated polarization from naïve (M0) to pro-inflammatory (M1) and anti-inflammatory (injury healing) (M2) phenotypes representing extremes of a multidimensional/spatial continuum of function. The relative roles of M1 versus M2 MØ in osteogenesis remain partially obscure. Several investigations indicate that M2 MØ enhance osteogenesis. MØ contributions to osteogenesis likely involve the serial function of the spectrum of MØ phenotypes. The bone healing process may involve a transition from M1 contributions followed by M2 contributions. The preliminary data demonstrates that the relative abundance of M1/M2 MØ is altered by MSC exosomes resulting in marked reductions in M1 MØ and reduced M1/M2 ratio (FIG. 19).

However, significant gaps in knowledge remain concerning the mechanism(s) by which MØ contribute to bone regeneration. The cellular interactions (e.g. MØ/MSC) in local environments involve both direct cell-cell and soluble factor signaling. In addition to growth factors, cytokines and chemokines, cells secrete exosomes (30-150 nm extracellular vesicles containing protein and miRNA cargo) that transfer this cargo as regulatory signals from parental to target cells. MSC exosome contributions to healing may be direct (targeting osteoprogenitors) and/or indirect (targeting immune cells). MØ exosomes are implicated in healing, osteogenesis and MSC osteoinduction. While it is known that MSC's immunomodulatory function involves exosomes, it was not fully know how MSCs direct MØ polarization or how MØ specifically target osteogenesis. It is herein hypothesized that MSC exosomes are regulators of the polarized population that contributes specific exosomes to control osteoinduction.

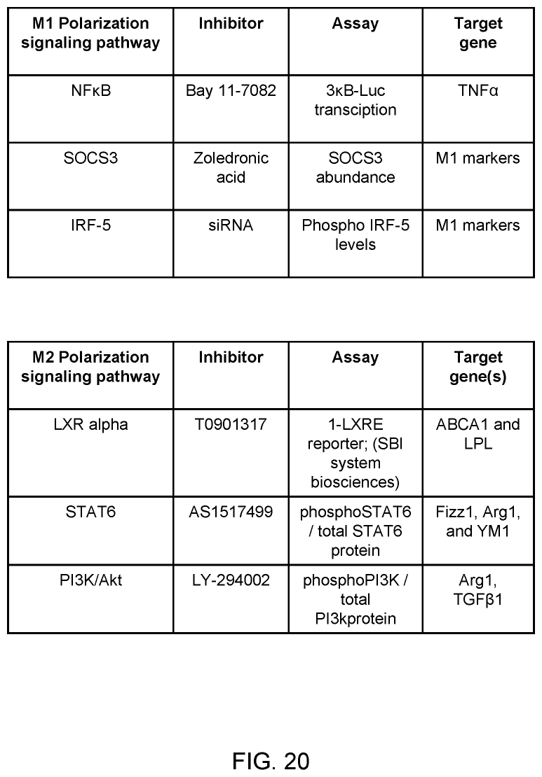

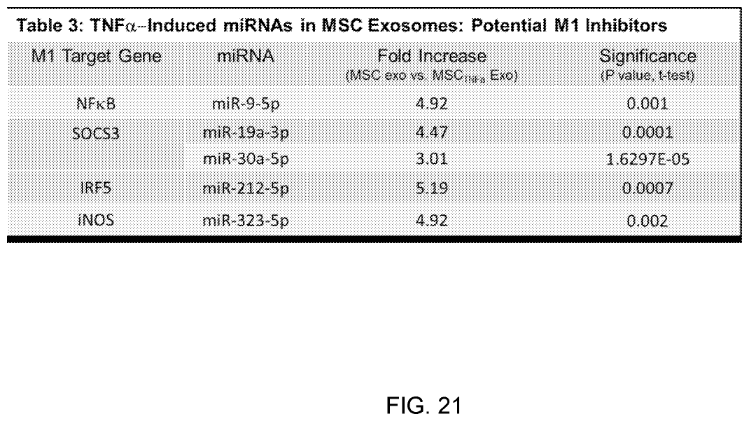

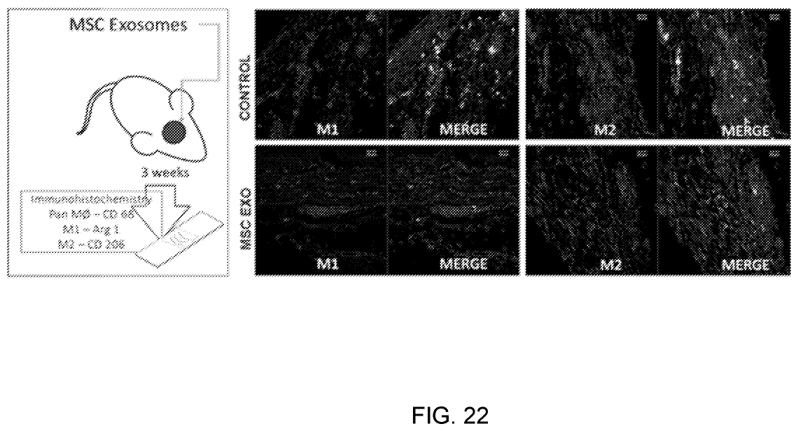

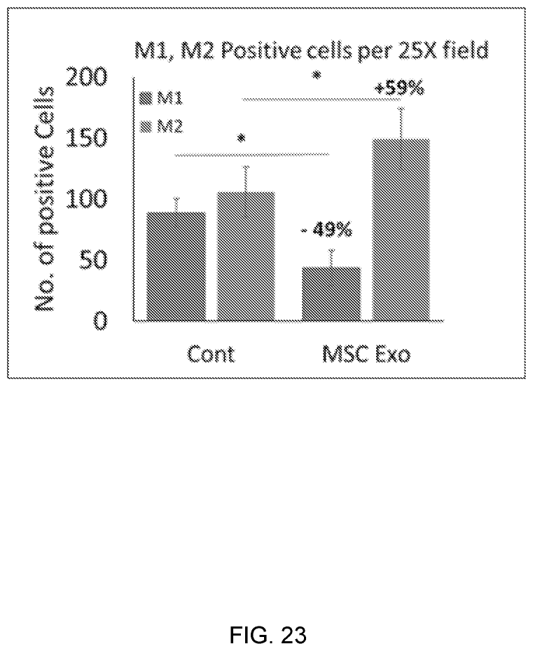

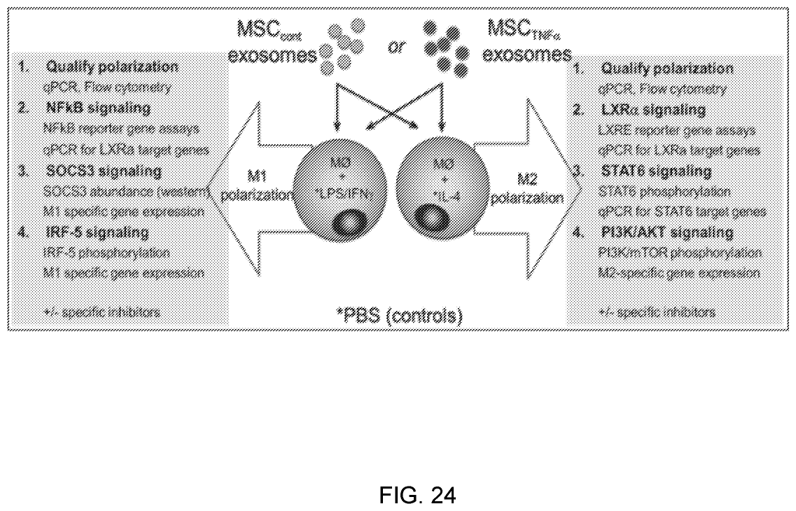

The preliminary data indicates that 1) MSC exosomes influence the relative abundance of MØ (↓M1/M2 ratio, FIG. 19), 2) inflammation-informed MSC exosomes contain miRNAs that control MØ polarization (FIG. 20 and FIG. 21), 3) M1 and M2 MØ exosomes differ in promoting bone regeneration (FIG. 22 and FIG. 23), and 4) MØ exosome miRNA cargos differ with M2 exosomes carrying osteoinductive miRNAs (FIG. 24, FIG. 20).

These data form a fundamental premise for the investigation of MØ exosome-mediated mechanisms acting in MSC mediated osteoimmunology. The MSC exosome miRNA-targeted mechanisms affecting MØ polarization are explored. It was confirmed that exosomes are specific and powerful agents for influencing—among many biological and pathological processes—osteogenesis.

The preliminary miRNA Seq data indicate that there are but a few miRNA unique to polarized MØ and it is possible to mechanistically characterize those functioning in osteoinduction. This approach has not been described nor exploited. Then, miRNAs and their targets are identified, and deployed for regulation of bone regeneration. Regarding clinical translation, exosomes (and miRNA cargo) can be readily produced from cultured cells, engineered to carry select miRNAs (and complementary drugs), are immune privileged and may be delivered in many carriers or directly to tissues.

General Methods

Cell culture: Primary mouse bone marrow MSC is isolated from 6-8 week old mice as previously described. Femurs and tibias are dissected from surrounding tissues. The epiphyseal growth plates are removed from dissected femurs and tibias and the marrow are flushed with α-MEM containing 100 U/mL of penicillin/streptomycin, and 10% fetal calf serum (FCS) with a 25G needle. Single cell suspensions are prepared by passing the cell clumps through an 18G needle followed by filtration through a 70-mm cell strainer. Cells are plated at a density of 2.5×10₆ cells/cm² in 75 mL culture flasks. After 4 days, one-half of the medium containing non-adherent cells is replaced with fresh medium. The phenotype of cultured MSC is characterized functionally by multi lineage differentiation using published culture conditions and is further defined by flow cytometry (CD44+, CD90+, CD45−) at the UIC RRC.

Primary mouse bone marrow MØ is isolated from the femurs and tibias of 6-8 week old mice. After cutting the proximal and distal epiphyseal plates, the marrow is flushed with 10 mL warm M199 media+10% FCS using a 28 gauge needle. After filtering cells through a 70 mm cell strainer, cells is carefully pipetted to a single cell suspension and then collected by centrifugation at 250×g for 10 minutes at room temperature. Cells are resuspended, counted, and plated on low adherence plastic (Costar) at 2×10₆ cells/mL in 6 well plates and supplemented with 20 ng/mL M-CSF. The plated cells are washed 2× in PBS every 2-3 days with replacement of M-CSF containing medium. At 6 days, the adherent MØ is collect using pre-warmed trypsin and their phenotype validated by staining and flow cytometry (F4/80+, CD 68+).

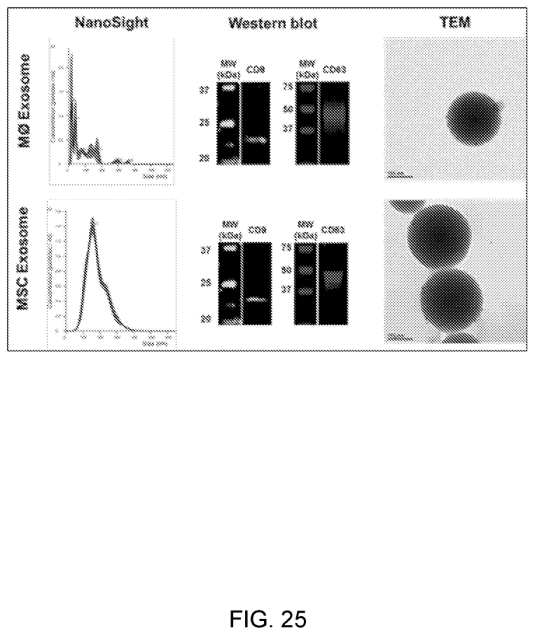

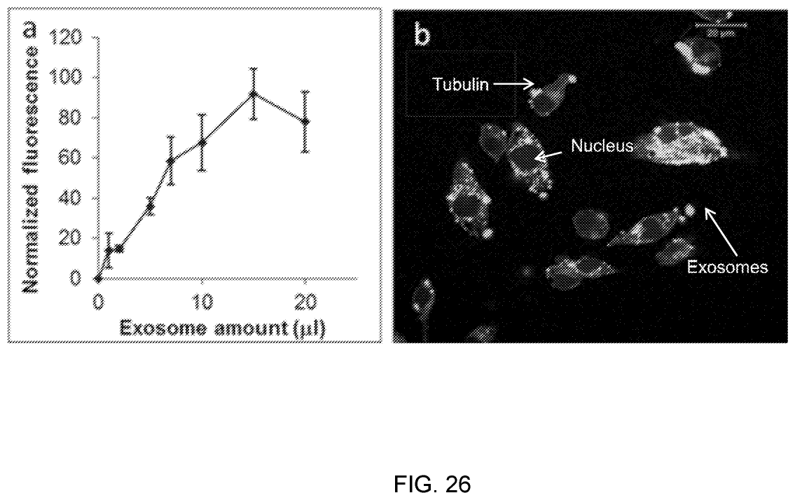

Isolation and characterization of exosomes: Exosomes are isolated and characterized by the published protocols and following standards developed for exosomal characterization. Exosomes are isolated from the culture medium of mouse bone marrow MSCs (MSC) and bone marrow derived MØ. One day prior to exosome isolation, the cell cultures are washed in PBS and cultured for 48 hours in serum free media. The exosomes from the culture medium are isolated using the ExoQuick-TC (System Biosciences) exosome isolation reagent as per the manufacturer's protocol. The isolated exosome suspension undergoes washing and buffer exchanges during the isolation procedure and is devoid of any measurable media constituents when purified. Exosomes are used in stock concentrations of 9×10₆ particles/mL and diluted based on saturation studies as previously reported. Cross-verification is performed by measuring RNA and total protein isolated from the exosome suspensions to ensure that RNA/protein concentration from the same volume of exosomes remained consistent. Then, the size heterogeneity of exosomes is determined using NanoSight (FIG. 25) and identify CD63 and CD9 protein by immunoblotting (FIG. 25). The presence of exosomes in the isolates is verified by transmission electron microscopy (TEM) (FIG. 25). For all exosome batches, immunoblotting is performed with exosome markers CD63 (Abcam, 1/1000) and CD9 (Abcam, 1/1000) antibodies (FIG. 26). Anti-tubulin antibody (Sigma, 1/10,000) is used in future as negative marker for intracellular proteins.

Fluorescent labeling of exosomes: Exosomes are stained using the Exo-Glow-Green labeling kit (System Biosciences) as per the previously published protocol. As a control, PBS not containing exosomes are subjected to labeling to control against non-specific staining. Exosomes are observed and quantified by immunofluorescence (FIG. 26).

Phenotype assessments by Real Time PCR: Osteoblastic differentiation and MØ polarization are monitored in various experiments at the level of mRNA expression using RT PCR. SYBRgreen-based assays are performed as previously reported using panels of OB- and MØ-specific primers and control primer pairs. Briefly, total RNA is isolated using the Qiagen RNA isolation kit, first strand cDNA synthesis is completed and gene specific primers is used to direct PCR amplification and SYBRgreen probe incorporation using a BioRad CFX96 thermocycler. Fold change is calculated using −ΔΔCT method. For most studies, n=4 is used for comparison using student's t-test. All cell culture based studies is conducted in 6, 12 or 24 well dishes with 4 replicates/group or time point. Experiments are repeated at least twice. Statistical analyses are performed as described below.

All animal breeding, care and treatment are conducted according to the UIC ACC approved protocols specific to this project and monitored by veterinarian staff of the UIC Biological Resource Laboratory. Surgeries are conducted under sterile conditions using intraperitoneal ketamine anesthesia (16 mg/ml, 80 mg/kg). Calvaria hair is removed and a full-thickness cutaneous incision and flap made to reveal the parietal and occipital bones. Mid-skull transcortical defects are created using a 3.5 mm trephine in a dental drill. Defects are filled with 3.5 mm diameter collagen scaffolds containing PBS, or exosomes from MSCs or M0, M1 or M2 MØ (described above). Additionally, scaffolds are treated with recombinant human Bone Morphogenetic Protein 2 (rhBMP2, 50 ng/scaffold) as positive controls. As NSAIDs may influence MØ function, buprenorphine is given subcutaneously (0.1 mg/kg body weight, BID) for pain relief according to the UIC BRL guidelines. Following 1-21 day healing periods, mice is euthanized, calvaria dissected of soft tissues, fixed in 4% paraformaldehye at 4° C. for μCT followed by histological processing. Routine husbandry procedures including cage cleaning, feeding and watering are conducted every other day.

Statistical analyses: For the proposed experiments, data obtained is presented as mean+/−SD. All comparisons between multiple groups are performed using ANOVA. Pairwise comparisons among groups are performed using Tukey's method. Individual pairwise comparisons are performed using student's t-test; the confidence interval is set at 95% (P<0.05). All quantitative studies using μCT data are performed using Matlab software and the results compared for significance using ANOVA. Quantification of histological data is performed by evaluating at least 5 regions/section and a total of 5 sections spanning the thickness of the embedded tissue resulting in a total of 25 images/sample. Statistical significance is calculated as stated above.

Power analysis: The number of animals used per group was based on the preliminary data and was determined by power analysis assuming 80% power, 0.5% significance, low standard deviation (<10%) and greater than 20% differences between experimental groups (e.g., μCT bone volume, number of cells). To define a 20% reduction in bone volume at p<0.05 and assuming 10% SD in measured volumes, a minimum of 6 animals is needed. The preliminary studies indicate error of 5-10%, and 10-20 differences among the groups. Eight animals per group (4 male/4 female to account for sex as a biological variable) provide sufficient power and permit loss of one animal per group.

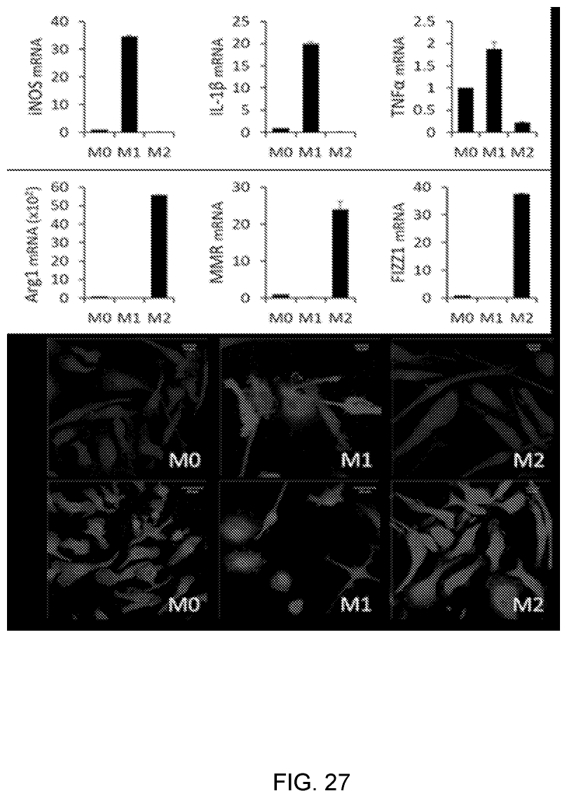

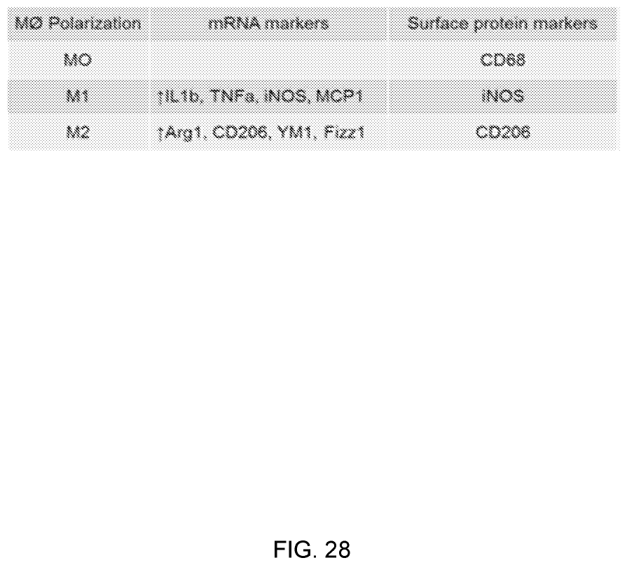

Mouse bone marrow derived MSCs and MSC exosomes (to be isolated, characterized and quantified as described above in general methods) are applied to MØ cultured in media, or media supplemented with 10 ng/ml LPS+1×10₃ U/ml IFNγ or 10 ng/ml IL-4 to direct M1 or M2 polarization, respectively (FIG. 27). MSC exosomes (or PBS control) are added to MØ plated in 12 well dishes (50,000 cells/well in 1 ml media) 4 hours prior to polarization using 3×10₈ exosomes/1 mL media. After 3 days, cultured MØ is washed with PBS, harvested by trypsinization, and placed in TriZol or fixed in 4% paraformaldehyde for RNA isolation and flow cytometry. MØ polarization is determined using qPCR and flow cytometry to identify polarization specific markers (FIG. 28). All experiments are conducted using 5 wells/experimental time point or exosome type.

To assess the impact of inflammation on MSC exosome signaling to MØ, parallel studies are conducted using exosomes of MSCs treated with 10 ng/ml TNFα for 18 hours (MSC_{TNFα}) to mimic the early inflammatory phase of bone injury. The preliminary studies indicate that MØ inflammatory cytokine expression is differentially altered by MSC_{cont} versus MSC_{TNF}α exosome treatment (FIG. 19). TNFα treatment of mouse MSCs alters their exosome cargo with increases in miRNA that have previously been shown to reduce MØ M1 polarization (FIG. 21). This new data is complimentary to knowledge that MSC TNFα pre-conditioning enhances MSC exosome production and their osteoinductive function.

In an initial effort to define the impact of inflammation-informed exosome miRNAs on MØ polarization, MØ is treated with either MSC_{cont} or MSC_{TNFα} exosomes and Antagomirs to each of the five miRNAs of increased abundance in the MSC_{TNFα} exosomes (FIG. 21). Antagomirs (and scrambled controls; Qiagen) are added to MØ in 24 well plates (1 mL media, n=5) at a concentration of 100 nM and incubated for 24-72 hours.

Subsequently, levels of target miRNAs are quantified by miR qRT-PCR and reported relative to snRNA-U6. In parallel, mRNAs for MØ and M1 polarization are quantified by qRT-PCR as described in general methods. It is expected that antagomir treatment ameliorate the effect of MSC_{TNFα} exosomes on MØ polarization.

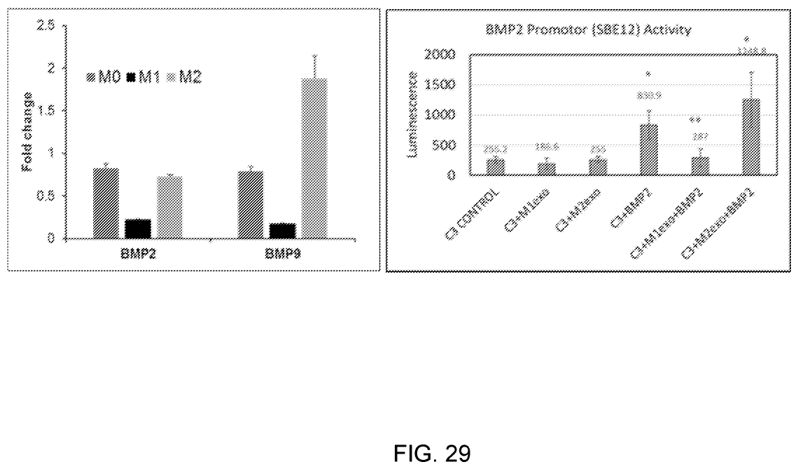

MSC miRNAs may play a key role in directing this shift to a regenerative MØ population. It was observed by immunohistochemistry that MSC exosome treatment in vivo reduces the ratio of M1/M2 MØ in healing calvaria (FIG. 22 and FIG. 23). The M1/M2 ratio was reduced from 0.84 to 0.29 (p<0.02). This reduction is consistent with M1 versus M2 effects on bone repair (FIG. 29).

MØ are stimulated with LPS/IFNγ or with IL-4 (or PBS control) to direct M1 or M2 polarization 4 hours following the addition of MSC exosomes (or PBS control). To study inflammation effects on MSC exosomes, both MSC_{cont} and MSC_{TNFα} exosome treatment of MØ are performed (+PBS control tx). Inhibitors (and/or siRNA knockdown) of defined polarization pathways are included to demonstrate exosome mechanisms for both M1 or M2 pathway-specific polarization (FIG. 24). Scrambled siRNAs, empty vectors and inhibitor vehicle controls are used in all studies.

MSC exosomal miRNA effects on M1 polarization: M1 polarization involves signaling via NF [symbol] B, SOCS3, and IRF-5. Primary MØ are treated+/−LPS/IFNγ with or without exposure to MSCcont or MSCTNFα exosomes. The NF [symbol] B, SOCS3, and IFR-5 pathways are interrogated by treatment with pathway-specific inhibitors to determine the influence of MSC exosome miRNAs on M1 signaling. Of note, SOCS3 has been identified as both an activator and inhibitor of M1 polarization, while NF [symbol] B and IFR-5 are known inhibitory pathways. MSCTNFα exosomes possess increased levels of miRNAs that inhibit these pathways (FIG. 20). Signaling is measured using well-defined specific assays. The impact of treatment on polarization is examined by qPCR measurement of polarization-specific gene expression (target genes).

An immunohistochemistry approach for characterization of the MØ M1/M2 populations was adopted in healing tissues of the mouse calvaria defect model (FIGS. 22, 23 and 30).

To compare the influence of MSC_{cont} versus MSC_{TNFα} exosomes on the polarized MØ populations in calvaria over the early time course of bone regeneration, single 3.5 mm diameter calvaria defects are created by trephine drilling in 8 mice (4 male/4 female). 3.5 mm diameter collagen scaffolds are hydrated in media and loaded by incubation for 1 hour at 37° C. in 50 (L of media containing 8.0×10₈ MSC exosomes or saline (based on saturation studies; in press). Following 1, 3, 7, 14 and 21 days, the calvaria is harvested and fixed in 4% paraformaldehyde for 24 hours. Following paraffin embedding, sectioning and processing for immunohistochemistry, 5-10 (m thick sections are stained for MØ specific antigens (MØ—F4/80/CD 68; M1—CD80/iNOS; M2—CD206/Arg-1) and counterstained with hematoxylin. Osteoprogenitors are stained with anti-RUNX2 anti-Osterix-, and anti-BSP—specific antibodies. Requisite secondary antibody control staining is performed. Within the defects, for each antigen, three sections from each of 8 mouse calvaria/experimental group is imaged at 20× and immunostained cells is counted/area. The average number of M0, M1 and M2 specific immuno-stained cells/area is calculated and compared statistically by Student's t-test (as shown in FIG. 22 and FIG. 23). The potential different temporal associations of M1 or M2 MØ numbers with osteoprogenitor abundance is examined by regression analysis (time vs. osteoprogenitor #).

To implicate miRNA function in the MØ polarization-dependent exosome effects on osteogenesis, MSC exosomes from DICER KO mice is included because DICER is required for miRNA biogenesis and function. DICER ablation in MSC by Runx2/Cre impaired bone formation, indicating the activity of Dicer dependent miRNAs in osteogenesis. Osx-cre/Dicer(flfl) mice are used in these studies. The effect of VVT and DICER mice MSC exosomes on M1 and M2 polarization (LPS+IFN-γ and IL-4 treatment respectively) is evaluated by qRT-PCR, flow cytometry of CD80 and CD206 and immunocytochemical detection of iNOS and Arg-1. MSC exosomes are further characterized by size (100-200 nm) and quantified using NanoSight.

Six groups of mice are treated with collagen scaffold grafting; 1) collagen only, 2) collagen+rhBMP2 (positive control), 3) collagen+WT MSC_{cont} exosomes, 4) collagen+MSC_{TNFα} exosomes, 5) collagen+Dicer MSC_{cont} exosomes, and 6) collagen+Dicer MSC_{TNF} exosomes. Eight C57/BL J6 mice (male (4) and female (4)) are treated per time point per group. This experiment is intended to define the impact of MSC exosomes—and inflammatory signaling of MSCs influencing exosomes—on the polarized MØ population and will correlate the M1/M2 phenotype with the relative abundance of osteoprogenitors in healing bone. 240 mice (5 time points×6 treatment conditions×n=8) are required (statistical plan provided in the general methods section).

Complementing the experiments described above, additional studies are conducted for 4 and 8 weeks to assess the impact of MSC exosomes on calvaria bone regeneration: 1) MSC_{cont} exosome or saline treated collagen is applied in defects of WT mice to demonstrate that MSC exosome enhance bone regeneration. 2) Additional studies in WT mice are conducted using MSC_{TNFα} exosomes to evaluate the possible inflammation-induced change in MSC exosomes affecting bone regeneration. 3) WT mice are treated with Dicer KO mouse MSC_{cont} and 4) with MSC_{TNFα} exosomes to demonstrate the role of exosomal miRNA. 5) To mechanistically explore the role of inflammation, 3 of the miRNAs identified within MSC_{TNFα} are applied using engineered exosomes. The 3 candidate miRNAs demonstrate marked M1 polarization or enhanced M2 polarization of MØ in vitro. 128 mice are needed to account for 2 time points, 8 exosome treatment groups, and n=8 mice (4 male+4 female)/group.

MØ and MØ exosome cargo varies with polarization to directly influence healing. Polarization is associated with unique miRNA cargo and M2-specific miRNA are implicated in osteogenesis (FIG. 17 and FIG. 18).

Treatment of mouse MSCs with MØ exosomes alters osteoinductive gene expression in a MØ polarity-specific manner (FIG. 29). As shown, MSC treatment with M1 exosomes reduced BMP2 and BMP9 expression and inhibited BMP2 induced transcription at the BMP2 responsive promoter (SBE12, FIG. 29 right). In contrast, M2 exosomes significantly potentiated BMP2-mediated signaling at the SBE12 promoter, despite no significant increase in BMP2 mRNA levels (FIG. 29 left).

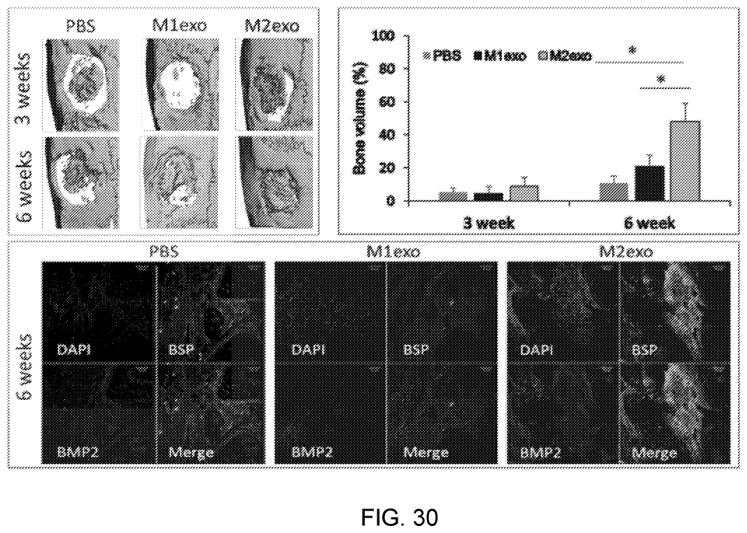

Further, when MØ exosomes in collagen scaffolds were engrafted in calvaria defects, the M2 MØ exosomes increased bone regeneration while M1 MØ impaired early regeneration (FIG. 30). These new data provide a basis for continued investigation of how MØ exosome miRNAs influence osteoinduction. While it is acknowledged that MØ exosomes may influence other resident cell types (“off target’), the congruence of in vitro osteoprogenitor responses and the in vivo result implicate these ‘on target” (BMP signaling responses to MØ exosome effects.

Recent analysis of miRNA among resting and LPS-treated MØ confirm that only a limited number of miRNAs differ among treated and untreated cells. This is consistent with the preliminary data (FIG. 25 and FIG. 28). Others have shown that highly expressed miRNAs are limited in number and comprise a high percentage of total miRNA reads.

Mouse primary bone marrow MØ is isolated and polarized to M1 and M2 phenotypes as described above. Their polarization is characterized at the level of gene expression (PCR) and surface marker phenotypes (flow cytometry) prior to their use. M1 and M2 polarizing MØ are cultured in 70 mL low adhesion flasks and media are collected for isolation of exosomes as described in general methods above. For these experiments, MØ isolated from three different donor mice (6 week old, 3 male/3 female) and the independent isolation of exosomes are achieved for subsequent miRNA-seq.

miRNA-seq QC and quantification: Adapters from raw reads are trimmed using trimmomatic to eliminate RNA sequences too long to be miRNA from the library. Trimmed reads are aligned directly to miRNA sequences obtained from MIRBASE using BWA ALN optimized for short read alignment. miRNA expression levels are quantified by counting the number of reads mapped to each miRNA sequence, and normalized to counts-per-million units for direct comparison between samples.

Differential expression: Differential expression statistics (fold-change and p-value) are computed using edgeR, on raw expression counts obtained from quantification. Importantly, edgeR allows multi-group analyses to prioritize which genes show the biggest effects overall, as well as pair-wise tests between sample conditions to specifically determine the context of the changes. In all cases, p-values are adjusted for multiple testing using the false discovery rate (FDR) correction of Benjamini and Hochberg. Significant genes will demonstrate an FDR threshold of 5% (0.05) in the multi-group comparison.

Clustering and visualization: Unsupervised clustering is used to determine predominant gene expression patterns that drive phenotype in an unbiased manner. Only miRNAs that show a statistically significant effect are first selected from the multi-group differential expression FDR. Hierarchical clustering of the gene expression levels is performed and plot the data in a heatmap. By visual inspection, gene sets with concordant expression patterns are determined, which putatively represent biological functions that are co-regulated during MØ polarization. After determination of the clusters of interest, self-similarity statistics within each cluster are computed to quantify the degree of separation.

Pathway analysis: The gene sets obtained from the hierarchical clustering and differential expression presumably represent cellular functions representing MØ polarization. A detailed perspective into different biological pathways enriched in each cluster is obtained using the Core Pathway Analysis database in Ingenuity Pathway Analysis. The statistical significance and enrichment of each pathway is compared between the miRNA clusters to compare how relevant osteoinductive functions are differentially regulated.

miRNA target analysis: A comprehensive miRNA target prediction using two tools, TargetScanMouse 7.2 (www.targetscan.org), and Diana Tools DIANA-microT (v5.0) (diana.imis.Athena-innovation.gr/Dianatools/index.php.) are used to anticipate the miRNAs impact on osteoinduction and osteogenesis. This is exemplified by the preliminary analysis conducted using the three specific miRNAs from MØ M2 exosomes (FIG. 18).

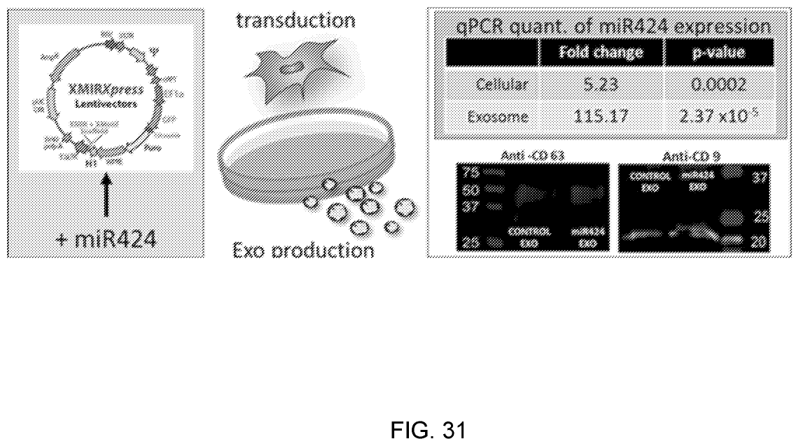

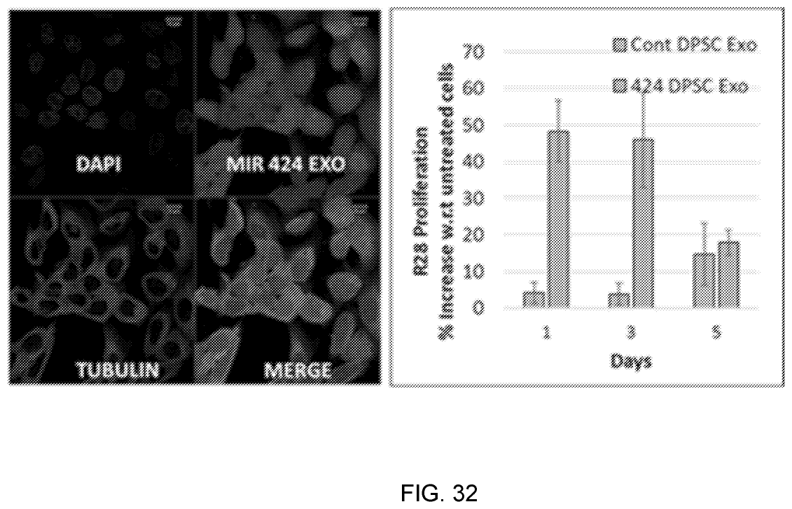

Overexpression and knockdown of miRNAs: miRNAs play a pivotal role in exosomal function. Therefore, specific manipulation of exosomal miRNAs may be used to control exosome functionality. Recent studies on miRNA sorting into exosomes have identified a target sequence in the 3′ end of miRNAs (GGAG; SEQ ID NO:8) that directs exosomal sorting and are available as expression systems (System Biosciences, XMIR expression system) directing miRNA into exosomes. MØ are genetically modified to express specific miRNAs selectively targeting into exosomes as demonstrated in FIG. 31 and FIG. 32.

Overexpression of M2 miRNAs is achieved by MØ transfection with lentiviral particles incorporating the select miRNA sequences preceded by the exosomal targeting sequence and subsequent selection for stable expression. Illustrating the current methodology, MSC exosomes were engineered for regenerative purposes by targeted expression of miR424, an anti-inflammatory miRNA of MSCs (FIG. 31 and FIG. 32). Using this approach, MØ is transduced with M2 MØ miRNA encoding XMIR (AXMIR for knockdown) plasmids and then selected for stable expression. Exosomes is isolated from these cell lines as described. The size distribution, presence of exosomal markers and endocytic properties of the modified exosomes is verified as per the standardized protocols (general methods). To assess the over expression or knockdown of miRNA in the engineered exosomes, total exosome RNA is isolated and qRT-PCR is used to evaluate the engineered exosome expression levels of the selected miRNAs with respect to control exosomes and vector-control exosomes. Increased miRNA levels with respect to control MØ exosomes (student's t-test, P<0.05) will denote success. The functionality of these engineered exosomes are explored as described below. Note the modification of parental cells does not affect exosome endocytosis in target cells (FIG. 31 and FIG. 32).

Gene expression, protein expression and osteoblastic differentiation in MØ exosome/miRNA—targeted MSCs: Osteoblastic differentiation of primary mMSCs are performed using standard procedures and assays. Briefly, primary mouse bone marrow MSCs is cultured with osteogenic media (OM) containing α-MEM supplemented with 15 FBS, 0.1 mM dexamethasone, 10 mM β-glycerophosphate, 50 mM ascorbate-2-phosphate, 100 U/mL penicillin, 100 mg/mL streptomycin, and 250 ng/mL amphotericin B. Cells grown in MSC medium (α-MEM containing 10% fetal calf serum, 100 U/ml of penicillin/streptomycin) are used as controls. Media is changed every 3 days and cultures are maintained for 28 days.

To define the possible impact of the selected M2 MØ miRNAs on mouse bone marrow MSC osteoblastic differentiation, P2 or P3 mouse MSC is cultured to 80% confluence (day 0) in 12 well culture plates and treated at day 0 with PBS, M2 exosomes or engineered MØ exosomes (9×10⁶/μL). OM+/−miRNA-engineered exosomes are changed every third day for 28 days. Assays for osteogenic differentiation include colorimetric assessment of alkaline phosphatase activity, calcium deposition using alizarin red staining, and qPCR analysis of osteogenic gene expression (RUNX2, OSX (SP7), BMP2, BMP2, BSP, DMP1 and OC). Differentiation assays are performed using n=5 wells/engineered exosome variable and per time point and analyzed by Student t-test (p<0.05). All assays are repeated using MSCs from three different mouse-derived MSC cultures. Results are compared to both OM only—and MØ exosome—treated MSC cultures.

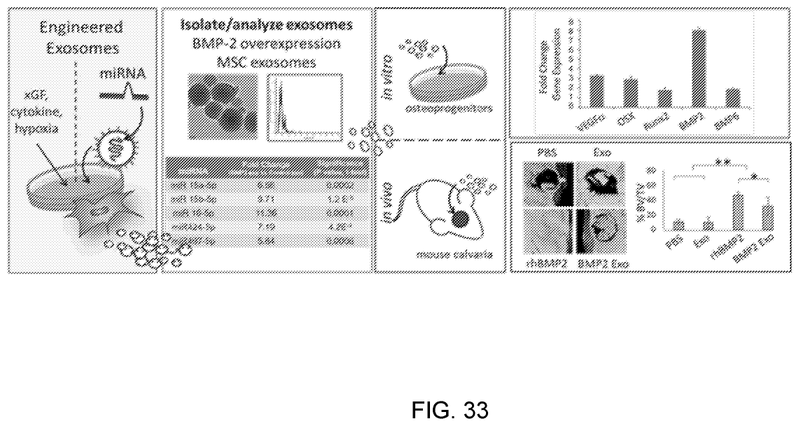

M2 MØ exosomes positively alter bone regeneration in the calvaria model and that MØ exosomes are effectively delivered to the calvaria defect using a simple expanded collagen scaffold (FIG. 30). Further, engineered exosomes enhance bone repair (FIG. 33).

Engineered MØ exosome engraftment: The miRNA₁, miRNA₂, and miRNA₃ are identified by the linear selection process involving miRNA seq, in silico targeting and validation, and cell culture osteogenesis assays detailed in sub aim 2b. These miRNAs are expressed in exosomes as described above. The engineered exosomes are isolated using ExoQuick-TC and quantified using NanoSight (general methods), and targeted miRNA expression is quantified by qRT-PCR. 3.5 mm calvaria defects are created by standard surgical techniques. The calvaria defects are grafted by placement of 3.5 mm collagen scaffolds, with or without MØ exosomes (4.0×10₈ exosomes/defect). Collagen scaffolds are hydrated with saline or saline with exosomes at 37° C. for 1 hour prior to surgical engraftment. 8 mice (4 male, 4 female) are treated per group and per time point using the following treatment groups: 1) collagen+saline, 2) collagen+M1 MØ exosomes (negative control, isolated from MØ treated with LPS+INFγ), 3) collagen+M2 specific miRNA₁ engineered exosomes, 4) collagen+M2 specific miRNA₂ engineered exosomes, and 5) collagen+M2 specific miRNA₃ engineered exosomes. Healing will occur 4 weeks to assess initial mineralized matrix formation and 8 weeks to assess the extent of bone repair. 80 mice (2 time points×5 groups×8 mice (4 male+4 female) are required for each of two repeated experiments (power analysis in general methods).

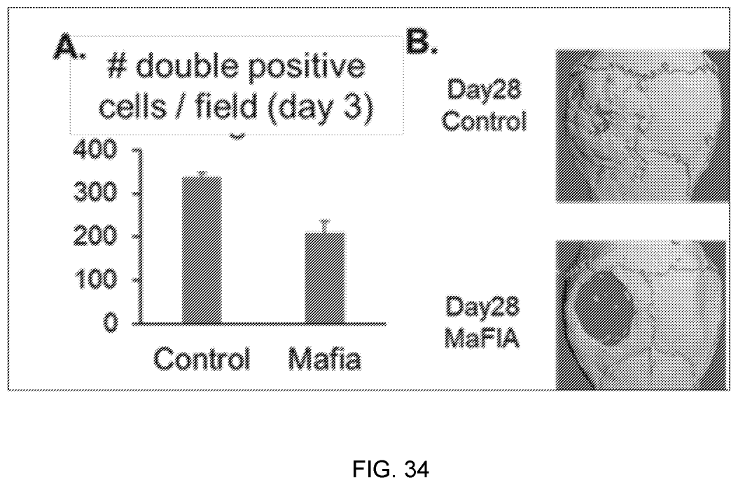

MØ exosome complementation in MØ depleted mice. Depletion of MØ in the MaFIA mouse reduces bone formation. The preliminary data shows that MØ reduction is associated with reduced bone healing in this model (FIG. 34). To directly implicate the M2 exosomes and the possible effects of specific M2 miRNAs in bone regeneration, a second series of calvaria regeneration studies are conducted treating calvaria defects using 4.0×10⁸ exosomes/defect in MØ depleted MaFIA mice. Quantification of bone regeneration is compared between groups as a function of the presence of M2 or engineered MØ exosomes in the presence or absence of AP20187 treatment. It is anticipated that M2 and engineered MØ exosomes will partially reverse the AP20187 mediated MØ ablation and related inhibition of bone regeneration by replacing key MØ exosomes and miRNA involved in signaling of osteoinduction and osteogenesis.

Osteogenesis within the calvaria defects is measured using standard methods for a) μ(CT-based morphometry, b) histology, c) immunohistochemistry using anti-Runx2 and anti BSP antibodies (the MSC marker Stro-1 does not identify mouse MSCs, and CD29 is expressed by MØ) and d) RT-PCR assessment of osteoblastic gene expression (see general methods).

Four- and 8-week time points are evaluated for bone regeneration. Studies are conducted in MaFIA mice treated with AP20187(MØ depleted) or saline (background control). Under each condition, the treatment groups are: 1) collagen scaffold (control), 2) collagen+M2 exosomes, 3) collagen+M2 specific miRNA₁ engineered exosomes, 4) collagen+M2 specific miRNA₂ engineered exosomes, and 5) collagen+M2 specific miRNA₃ engineered exosomes. Eight mice (4 male/4 female) are used/group. 160 mice are required [(n=8 mice (4 male+4 female)×2 time points×5 groups)+/−AP20187] for each of two repeated experiments.

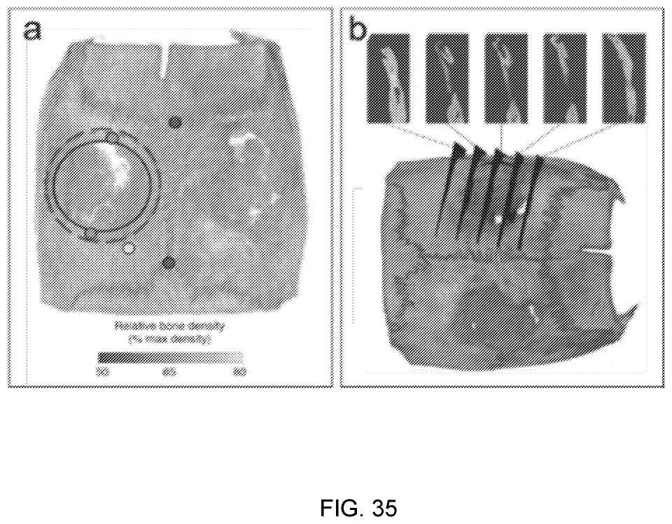

The μ(CT-based morphometry is based on a novel Matlab script that automatically calculates the volume of mineralized tissue within a fixed 3.5 mm diameter cylindrical volume of interest (FIG. 35). This reduces dramatically the labor of manual segmentation. The μ(CT data is imported into the Matlab software using custom scripts and stored as voxels of greyscale values. The data are then segmented on grey scale values and relative bone density calculated based on maximum density of intact calvarium. The defect boundaries and the center point are then set manually and the software was programmed to create an ROI of the defect diameter (˜3.5 mm) cutting across the z plane. For the experimental and control regions, the regenerated volume was determined by summation of the greyscale values within the ROI and percentage regeneration was calculated based on the total volume of the cylindrical ROI. To visualize the 3D distribution of bone density, a modified version of the Matlab function vol3d_v2 (version 1.2.2.0) was used to create 3D renderings using orthogonal plane 2D texture mapping techniques. The volume of newly formed bone within the implanted scaffolds is quantified and expressed as bone volume over total volume (BV/TV %). CT analysis will include bone volume (BV/TV %).

### Example 3: Neuronal Regenerative Exosomes

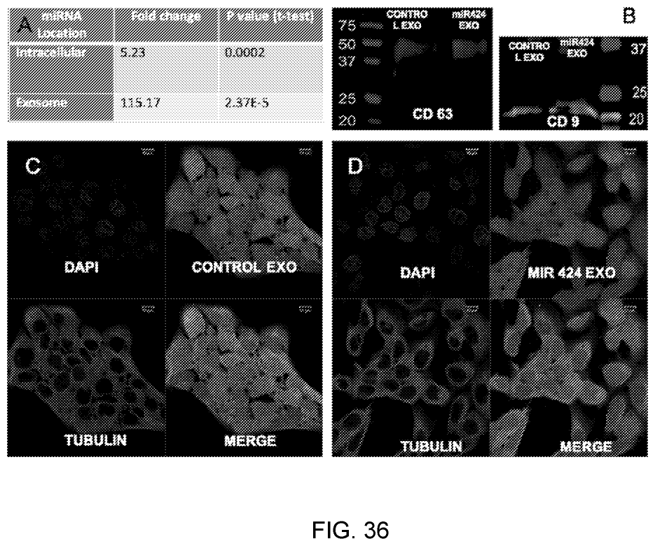

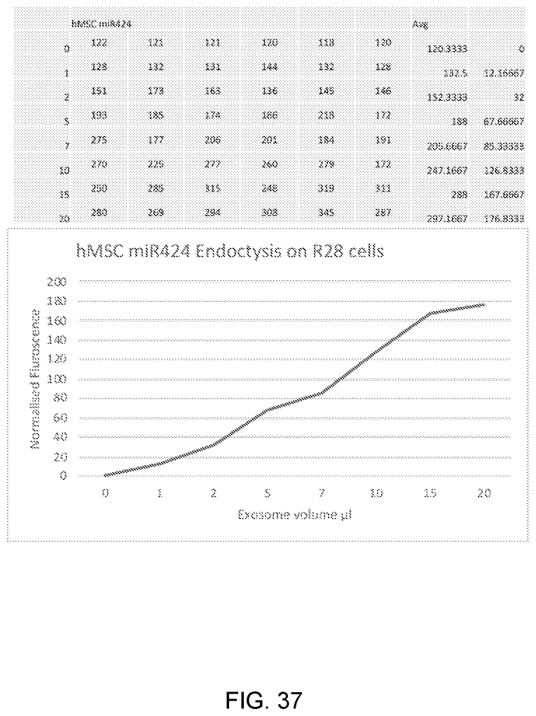

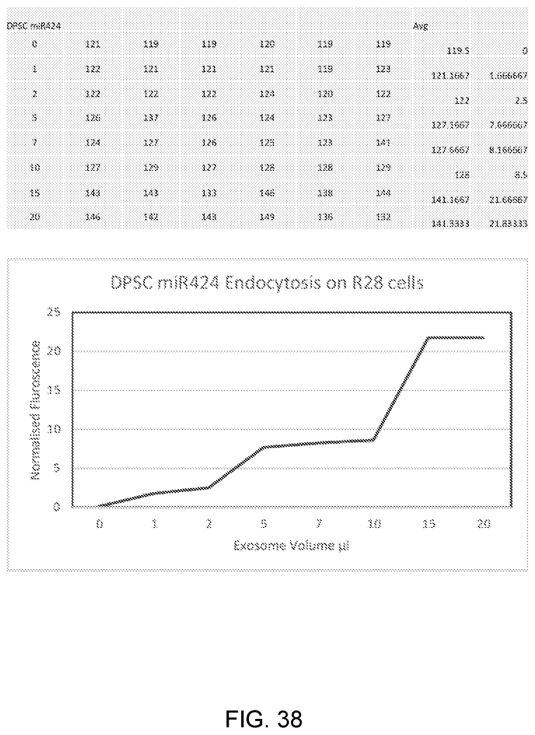

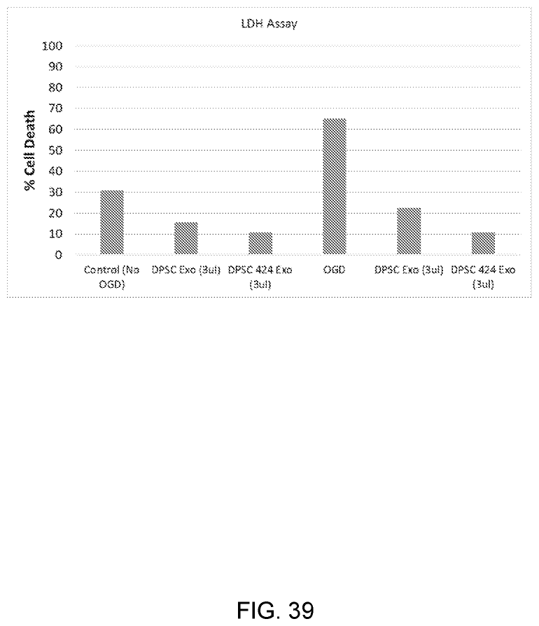

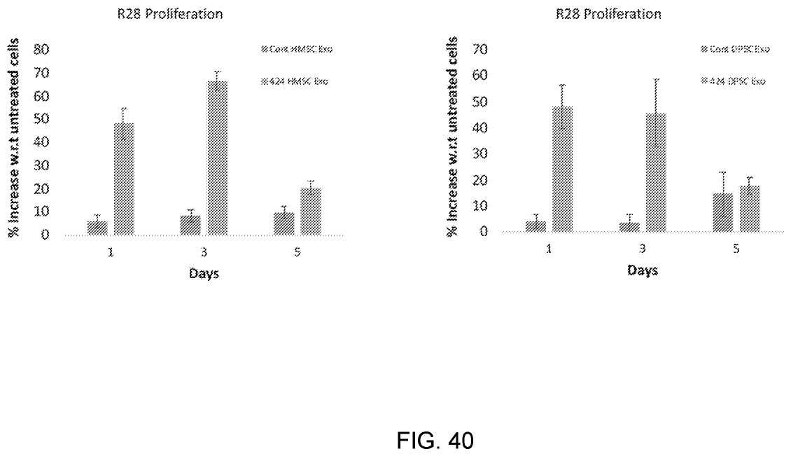

Exosome-specific exprssion of miR424 was achieved, and the exosomes were successfully endocytosed (FIG. 36). Human bone marrow derived MSC and DPSC (dental pulp stem cell) were genetically modified to overexpress miR 424 with an exosome targeting sequence. The resulting exosomes were evaluated for their ability to be endocytosed by retinal neuronal cell line R28 (FIGS. 37 and 38). To evaluate the function of these engineered exosomes under ischemic conditions, ischemic conditions were mimicked in R28 retinal cells by subjecting them to oxygen and glucose deprivation (OGD). To test the hypothesis if exosomes can rescue R28 cells from OGD-mediated cell death, the R28 cells were subjected to OGD conditions for 6 h and later were treated with exosomes for about 18 hours. The cytotoxicity was measure by release of LDH (LDH is an enzyme that is released when cells are dying) by the cells. As seen in FIG. 39, OGD conditions caused more than 50% of cell death. Conversely when same were treated with DPSC exosomes showed significant reduction in % cell death as compared to cells with absence of exosomes. The same experiment was performed using DPSC miR424 derived exosomes. Similar results were obtained. When compared, DPSC miR424 derived exosomes proved more effective than DPSC exosomes (FIG. 40). Also, conditioned media depleted of exosomes were tested and fewer protective effects were seen implying that the protective effects are due to the presence of exosomes (data not shown).

### Example 4: Mesenchymal Stem Cell-Derived Extracellular Vesicles and Retinal Ischemia-Reperfusion

Retinal ischemia is a major cause of vision loss and impairment and a common underlying mechanism associated with diseases such as glaucoma, diabetic retinopathy, and central retinal artery occlusion. The regenerative capacity of the diseased human retina is limited. Previous studies have shown the neuroprotective effects of intravitreal injection of mesenchymal stem cells (MSC) and MSC-conditioned medium in retinal ischemia in rats. Based upon the hypothesis that the neuroprotective effects of MSCs and conditioned medium are largely mediated by extracellular vesicles (EVs), MSC derived EVs were tested in an in-vitro oxygen-glucose deprivation (OGD) model of retinal ischemia. Treatment of R28 retinal cells with MSC-derived EVs significantly reduced cell death and attenuated loss of cell proliferation. Mechanistic studies on the mode of EV endocytosis by retinal cells were performed in vitro. EV endocytosis was dose- and temperature-dependent, saturable, and occurred via cell surface heparin sulfate proteoglycans mediated by the caveolar endocytic pathway. The administration of MSC-EVs into the vitreous humor 24 h after retinal ischemia in a rat model significantly enhanced functional recovery, and decreased neuro-inflammation and apoptosis. EVs were taken up by retinal neurons, retinal ganglion cells, and microglia. They were present in the vitreous humor for four weeks after intravitreal administration, with saturable binding to vitreous humor components. Overall, this study highlights the potential of MSC-EV as biomaterials for neuroprotective and regenerative therapy in retinal disorders.

Age related macular degeneration, diabetic retinopathy, and glaucoma are the leading causes of irreversible blindness in Western countries, predicted to affect approximately 200 million people by 2020. Retinal ischemia and cell death resulting from, among other mechanisms, apoptosis and inflammation, are the hallmark events in the pathogenesis of the resulting visual loss. Current therapy focuses upon arresting disease progression using intraocular injections (e.g., anti-VEGF), eye drops, or surgery. Limitations of these treatments motivate studies of alternatives with greater safety margin, and higher likelihood of reaching the retinal target cells.

Successful strategies for enabling repair and regeneration of injured or diseased tissues should overcome the limitations of using morphogens and growth factors and rely on biomimetic strategies that minimize immunological and oncogenic consequences. In this regard, stem cell therapy using mesenchymal stem cells (MSCs) serves as an attractive option. MSCs are multipotent cells with regenerative and immunomodulatory properties. It has been previously reported that MSCs exhibit a robust neuroprotective effect, as does their conditioned medium, in an in vivo rat model of retinal ischemia-reperfusion injury. In the eye, stem cell-based retinal cell replacement is a highly encouraging approach to trigger neuroprotection and/or regeneration. However, low cell integration and aberrant growth, among other factors, limit its promise.

On the other hand, mounting evidence suggests that most MSC effects are paracrine in nature and are mediated by MSC derived extracellular vesicles (EVs). Several groups have reported on the regenerative potential of MSC-EVs in soft and hard tissue regeneration. Therefore, it can be possible to avoid the limitations and complications of stem cell therapy in the eye by using MSC derived EVs as biomimetic agents to aid neuroprotection and regeneration. This approach is made feasible by the fact that apart from possessing neuroprotective and regenerative properties, MSCs are also prolific producers of EVs. Therefore, MSCs can prove to be an ideal source for therapeutic EVs that can be applied as naturally occurring biomaterials. Additionally, published studies show that EVs decrease neuronal cell death after hypoxia/ischemia in vitro and in vivo, stimulate axonal growth, and are anti-inflammatory and immunomodulatory, supporting a potential treatment role in retinal diseases. Therefore, an aim of this study was to test the hypothesis that MSC-EVs attenuate injury produced by hypoxia and ischemia in the retina.

EVs are integral to intercellular communication, interacting with recipient cells by three main mechanisms which resemble viral entry: 1) Binding surface receptors to trigger signal cascades, 2) internalization of surface-bound EVs via endocytosis, phagocytosis, or macro-pinocytosis, and 3) fusion with the cell to deliver material directly to the cytoplasmic membrane and cytosol. Presently, there is a foundational knowledge gap with respect to the endocytosis of MSC-EVs by retinal cells and their mechanisms of entry. Uptake can depend upon proteins on the EV surface and the target cell. A logical hypothesis is that cells use unique, and likely multiple, means to internalize EVs, e.g., integrins are necessary for EVs internalization in dendritic cells, macrophages, and heparin sulfate proteoglycans (HSPGs) for entrance into cancer cells. Moreover, clathrin- and caveolin-mediated pathways can be involved. Therefore, one of the aims of this study was to evaluate the endocytic mechanism of MSC-EVs by retinal cells. These mechanistic studies help in developing a foundational knowledge of MSC-EV functionality in neuronal cells that can be exploited to promote enhanced delivery for engineered EVs as well as to facilitate cell-type specific targeting.

Compared to studies of neuronal injury in vivo, retinal neurons and other cells in the retina such as glial cells are more readily accessible by injection directly into the vitreous humor. Thus the retina is ideal as a window into the brain for studies of EV mechanisms and therapeutics that targets neuroprotection and regeneration. This route is also commonly used in the treatment of retinal disease and EV therapeutics should be optimized to use the intravitreal injections advantageously. However, the principles governing EV transit within tissues under normal and pathological conditions are poorly understood and are necessary to be determined in order to reach the full potential of EVs as effective biomaterials for ocular therapy.

Most pre-clinical studies use systemic administration of EVs. This is a low efficiency method as much of the injected dose is distributed outside of the target organ. For the retina, EVs delivered into the vitreous humor are expected to gain direct access to the inner retina cells including the retinal ganglion cells (RGCs). The vitreous humor is predominantly comprised of collagen and hyaluronic acid along with a network of extended random coil molecules that fills in the meshes of the collagen fiber network. However, studies utilizing intravitreal injections of EVs have not focused on their interactions with the vitreous humor, their endocytic mechanisms and distribution within the eye. This knowledge is vital for understanding EV dynamics in the intraocular space and provides a foundational knowledge for nanoparticle-based biomaterials movement in this environment. Based on the earlier observation that MSC-EVs can bind to type I collagen, it was hypothesized that the vitreous humor proteins can bind to EVs and serve as a reservoir for EVs prolonging their availability to retinal cells.

Overall, this study aimed to evaluate the use of MSC-derived EVs as biomimetic agents for neuroprotection/regeneration following ischemic insult or injury using the eye as a model system and characterizing the fundamental aspects of EV behavior within the eye and the retina in particular.

### Materials and Methods

Isolation of human bone marrow mesenchymal cell derived EVs: Human MSCs (hMSCs) were purchased from American Type Culture Collection (ATCC, Manassas, Va.) and cultured in α-MEM supplemented with 20% FBS, 1% L-Glutamine, and 1% antibiotic-anti-mycotic solution (all from GIBCO, Thermo-Fisher). They were seeded to confluence cultured for 4 weeks. Subsequently, EVs were isolated from the culture medium. Briefly, cultures were washed with serum-free medium and cultured 48 h in the same medium under normoxic (21% O₂, 37° C.) conditions. Conditioned medium was collected and centrifuged to remove whole cells and debris. After filtration with a 0.22-μm pore filter, supernatant was transferred to a 100-kDa molecular weight cut-off ultra-filtration conical tube (Amicon Ultra-15, Millipore, Burlington, Mass.), and centrifuged (3,000×g) at 4° C. for 45 min. EVs were isolated from the concentrated conditioned medium using Exo Quick-TC EV Precipitation Solution (System Biosciences, Palo Alto, Calif.). Isolated EVs were suspended in PBS, the suspensions normalized to cell number from the tissue culture plate from which they were isolated, and diluted such that 100 μl of suspension contained EVs isolated from 1 million cells. Cross-verification was performed by measuring RNA and total protein from EV suspensions to ensure that RNA/protein concentration from the same volume of EV remained consistent.

Characterization of MSC-EVs using electron microscopy, nanoparticle-tracking analysis, and Western blotting: MSC-EVs isolated from the conditioned medium were characterized for size, morphology, and the specific exosome surface marker CD63 by transmission electron microscopy (TEM). CD63 and additional exosome surface markers were also examined using immunoblotting. Nanoparticle Tracking Analysis (NTA) by Nanosight (LM10-HS, Malvern, Westborough, MA) measured MSC-EV concentrations and particle size to confirm the composition and consistency of the MSC-EV preparations.

MSC-EVs were adsorbed onto carbon-Formvar film grids and fixed in 2% glutaraldehyde/PBS at pH 7.4. Morphology was observed by TEM (80 kV, JEM-1220 TEM, JEOL, Peabody, MA), following staining with 2% phosphor-tungstic acid. For immunogold labeling, the MSC-EVs bound to the grids were permeabilized in 0.5% Triton X-100/PBS, then blocked with 5% BSA/PBS. The MSC-EVs were incubated for 2 h at room temperature in mouse monoclonal anti-CD63 (Abcam, Cambridge, Mass., 1/100). Grids were washed three times and then incubated 1 h at room temperature in gold-labeled secondary antibody (1/2000, Abcam). The grids were then washed, dried and imaged using a JEOL JEM-3010.

For immunoblotting, the MSC-EV pellets were lysed in 1×RIPA buffer with protease and phosphatase inhibitor cocktail. Lysates were centrifuged at 4° C. and protein concentrations measured using a protein assay kit (Pierce, Rockford, Ill.) Equal amounts of protein per lane (10 μg) were diluted with SDS sample buffer and loaded onto gels (4%-20% or 16%; Invitrogen-Thermo Fisher). Proteins were electroblotted to polyvinylidene difluoride membranes (Immobilon-P; Millipore, Bedford, Mass.) with efficiency of transfer confirmed by Ponceau S Red (Sigma, St Louis, Mo.). Nonspecific binding was blocked with 5% nonfat dry milk in Tween-Tris-buffered saline. Membranes were incubated overnight at 4° C. with primary antibodies: anti-CD81 (rabbit polyclonal, Abcam, 1/250), anti-CD63 (rabbit polyclonal, Abcam, 1/250), anti-CD9 (mouse monoclonal, Abcam, 1/250), and anti-α-HSP70 (rabbit polyclonal, System Biosciences, 1/1000. Anti-rabbit horseradish peroxidase (HRP)-conjugated (goat IgG; Jackson Immuno Research, West Grove, Pa.), or anti-mouse HRP-conjugated (sheep IgG; Amersham, Buckinghamshire, UK) secondary antibodies were applied at 1:20,000. Chemiluminescence was developed with a kit (Super Signal West Pico; Pierce). Protein bands were digitally imaged with a LICOR Odyssey (Lincoln, Nebr.).

### Fluorescent Labeling of MSC-EVs:

To image MSC-EVs in vivo and in vitro, isolated EVs were labeled with green fluorescent-tagging reagent Exo-Glow Protein (System Biosciences), which labels intra-exosomal proteins fluorescently. Briefly, MSC-EVs were suspended in PBS and incubated with Exo-Green Protein for 10 min at 37° C. followed by 30 min incubation on ice. Labeled MSC-EVs were precipitated by adding Exo Quick-TC and centrifuged for 30 min at 14,000×g. The obtained pellet was re-suspended in PBS.

Retinal cell line R28 culture: Retinal cell line R28 was purchased from Kerafast (Boston, Mass.) and cultured according to the supplier's instructions. R28 is an adherent retinal precursor cell line derived from postnatal day 6 Sprague-Dawley rat retina immortalized with the 12S E1A gene, and has been used previously in studies on oxidative stress in retinal cells. The 12S E1A gene was introduced via an incompetent retroviral vector; therefore, the cells produce no infectious virus. The cells have been passaged 200 times thus far, and show no signs of senescence. The heterogeneity of this cell line provides a diversity of cell types simulating in vivo retina and offers differentiation potential as an additional test of viability. Cells were cultured in DMEM with 10% serum (420 ml DMEM incomplete, 15 ml 7.5% sodium bicarbonate, 50 ml calf serum, 5 ml MEM non-essential amino acids, 5 ml MEM vitamins, 5 ml L-glutamine (200 mM) and 0.625 ml Gentamicin (80 mg/ml), with pH adjusted to 7.4.

In vitro oxygen glucose deprivation model: As an in vitro model of retinal ischemia, an oxygen-glucose deprivation (OGD) in R28 cells was used. R28 cells were plated to reach 70% confluence in normal medium. For OGD, cells were cultured in glucose-free medium and subjected to hypoxia (1% O2, 5% CO2) for 24 h. Cells were then re-oxygenated (21% O2, 5% CO2) for another 18 h, then assayed for lactate dehydrogenase (LDH, Promega, Madison, Wis.), and cell proliferation (ethynyl-deoxyuridine (EdU) assay followed by flow cytometry). Cytotoxicity was assayed by using Sytox non-radioactive cytotoxicity assay kit (Promega). Briefly, culture supernatant samples from normoxic and OGD cells treated with MSC-EVs were transferred to a 96 well plate and equal volume of Sytox reagent was added, incubated 30 min at room temperature, and absorbance measured at 490 nm. Percentage cytotoxicity was calculated from LDH release into the supernatant.

We used Click-iT EdU kit from Thermo-Fischer for measuring cell proliferation. Cells were labeled with EdU at the end of OGD and subjected to click reaction. The fluorescent signal generated by Click-iT EdU was detected by logarithmic amplification and analyzed by flow cytometry with a CyAn 2 Bench-top Analyzer (Beckman-Coulter, Brea, Calif.).

### Endocytosis Experiments:

For imaging, R28 cells were seeded onto glass coverslips in 6-well tissue culture plates. At 24 h post-seeding, 50 μl of fluorescently labeled MSC-EVs (corresponding to EVs isolated from 500,000 hMSCs) or PBS was added to the culture medium and incubated for 1 h at 37° C. The PBS control was subjected to a similar labeling procedure as the EV suspension prior to being used in the experiment. After each experiment, coverslips were washed in PBS three times, fixed in 4% neutral buffered formalin, and immuno-labeled using anti-tubulin (1/5000, Sigma), anti-clathrin (1/500, Santa Cruz Biotechnology, Santa Cruz, Calif.), or anti-caveolin-1 (1/1000, Santa Cruz). Slides were imaged using a Zeiss (Thornwood, N.Y.) LSM 710 confocal microscope or Zoe fluorescent imager (BioRad, Hercules, Calif.).

Quantitation of endocytosis and dose-dependency experiments were performed in 96 well ELISA plates, with 10,000 R28 cells per well. At 24 h post seeding, increasing amounts of MSC-EVs were added and incubated for 1 h at 37° C. For blocking experiments, 20 μI of MSC-EVs were used per 20,000 cells (2× saturation). Cells were pre-treated with either heparin (0, 5 and 10 μM, Sigma), RGD (Arg-Gly-Asp peptide, 0, 0.5, 1, and 2 mM, Abcam), MBCD (Methyl-β-cyclodextrin, 0, 2.5, 5 mM, Sigma), or incubated at 4° C. for 1 h followed by incubation with the MSC-EVs. The experiments were conducted in quadruplicate. Wells were washed 3 times in PBS, fixed using 4% neutral buffered formalin, and the fluorescence measured using a BioTek (Winooski, Vt.) 96 well plate reader equipped with the appropriate band pass filter sets.

In vivo rat model of retinal ischemia: Procedures conformed to the Association for Research in Vision and Ophthalmology Resolution on the Use of Animals in Research. Male Wistar rats (200-250 gm, Harlan, Indianapolis, Ind.) were maintained on a 12 h on/12 h off light cycle. For retinal ischemia, rats were anesthetized with ketamine 100 mg/kg, and xylazine, 7 mg/kg intraperitoneally (i.p.). After sterile preparation, and working under an operating microscope, a 30-gauge, ⅝-inch metal needle (BD Precision Glide, Becton-Dickinson, Franklin Lakes, N.J.) was placed with its tip inside the anterior chamber of the eye. The needle was connected by plastic tubing via a three-way stopcock to a pressure transducer (Trans-pac, Hewlett-Packard) and an elevated bag of balanced salt solution (BSS; by sterile technique BSS was transferred from its bottle (Alcon, Ft Worth, Tex.) to an empty 1000 ml 0.9% saline plastic bag. Intraocular pressure (10P), continually displayed on an anesthesia monitor (Hewlett-Packard HP78534C), was increased to 130-135 mm Hg for 55 min by pressurizing the bag (Smiths Medical Clear Cuff, Minneapolis, Minn.). The eyes were treated with topical Vigamox (0.5%; Alcon), cyclomydril (Alcon) and proparacaine (0.5%; Bausch & Lomb, Bridgewater, N.J.). Temperature was maintained at 36-37° C. using a servo-controlled heating blanket (Harvard Apparatus, Holliston, Mass.). Oxygen saturation of the blood was measured with a pulse oximeter (Ohmeda-GE Healthcare, Madison, Wis.) on the tail. Supplemental oxygen, when necessary to maintain O₂ saturation>93%, was administered with a plastic cannula placed in front of the nares and mouth.

Electroretinography: For baseline and post-ischemic follow-up electroretinography (ERG), rats were dark adapted and were injected i.p. with ketamine (35 mg/kg) and xylazine (5 mg/kg) every ˜20 min to maintain anesthesia. Custom Ag/AgCl electrodes were fashioned from 0.01 inch Teflon-coated silver wire (Grass Technologies, West Warwick, R.I.). Approximately 10 mm was exposed and fashioned into a small loop to form the corneal/positive electrodes while ˜20 mm was exposed to form a hairpin loop, the sclera/negative electrodes looped around the eye. To maintain moistness of the cornea and electrical contact, eyes were treated intermittently with Goniosol (Alcon). Electrodes were referenced to a 12 mm×30-gauge stainless steel, needle electrode (Grass) inserted ⅔ down the length of the tail. Stimulus-intensity ERG recordings were obtained simultaneously from both eyes using a UTAS-E 4000 ERG system with a full-field Model 2503D Ganzfeld (LKC Technologies, Gaithersburg, Md.).

The ERG a- and b-waves were expressed as normalized intensity-response plots with stimulus intensity (log cd·s/m²) on the X-axis, and corresponding percent recovery of baseline on the Y-axis. Recorded amplitude, time course, and intensity were exported and analyzed in Matlab 2011a (MathWorks, Natick, Mass.). ERG waveform recovery after ischemia was corrected for day-to-day variation and reference to the non-ischemic eyes. In vivo administration of MSC-EVs, and MSC-EV depleted conditioned medium into the eyes:

MSC-EV-depleted conditioned medium was prepared by isolating MSC-EVs from the medium as described above and served as control in addition to PBS. The conditioned medium was centrifuged, filtered to remove cells and debris, and concentrated using 10-kDa molecular weight cut-off ultra-filtration conical tubes (Amicon Ultra-15) by centrifuging at 3,000×g at 4° C. for 45 min. MSC-EVs were isolated as described above. Supernatant without MSC-EVs was evaluated for pH, and for protein concentration using a protein assay kit (Pierce). Normoxic MSC-EV-depleted conditioned medium (10 μg protein/4 μl), MSC-EVs (4 μl of 1×10⁹ particles/ml), or PBS (4 μl) were injected into the vitreous humor of both the ischemic (right) and non-ischemic (left) eyes, 24 h after retinal ischemia (4 μl is the maximal safe volume for injection into the vitreous humor in rats). The normal/non-ischemic left eye served as the control eye. Rats were subjected to ERG recordings at baseline, prior to ischemia, and at seven days post injections, i.e., 8 days after ischemia.

### Evaluation of Apoptosis and Inflammatory Markers in MSC-EV Injected Retinae:

Retinal tissue was homogenized with a Bead-Bug Micro-tube Homogenizer (Midwest Scientific, Valley Park, MØ) in RIPA buffer (Cell Signaling Technology, Danvers, Mass.) containing protease and phosphatase inhibitors. Lysates were centrifuged at 4° C. and protein concentration measured using a BCA protein assay kit (Pierce). Equal amounts of protein (15 μg) were loaded onto 10% sodium dodecyl sulfate-polyacrylamide gel electrophoresis gels, transferred onto nitrocellulose membranes and Western blotting was performed. Membranes were probed with anti-IL-6 (Santa Cruz, mouse monoclonal, 1/500, anti-TNF-α Santa Cruz, mouse monoclonal, 1/500), and anti-cleaved caspase-3 (Cell Signaling, rabbit polyclonal, 1/1000) primary antibodies. IL-6 and TNF-α are markers of inflammation, and caspase-3 of apoptosis gene-related expression. Band density was calculated using densitometry with macros in ImageJ (https://imagej.nih.gov/ij/docs/guide/user-guide-USbooklet.pdf) where each protein was normalized to anti-β-actin.

Fundus imaging and in vivo tracking of MSC-EVs in the eye: To track MSC-EVs in vivo, the EVs were labeled with Exo-Glow Protein prior to intravitreal injection. The labeled MSC-EV pellet was suspended in PBS and injected (4 μl of 1×10⁶ particles/ml) 24 h post-ischemia into the mid-vitreous under direct vision using an operating microscope, in both normal and ischemic eyes. For in vivo real-time imaging, rats were injected i.p. with ketamine (35 mg/kg), and xylazine (5 mg/kg). Pupils were dilated with 0.5% tropicamide (Alcon), and cyclomydril. Fluorescent fundus images were obtained using a Micron IV Retinal Imaging Microscope (Phoenix Research Labs, Pleasanton, Calif.), at 1, 3, 7, 14, and 28 days after injections into the vitreous humor.

Fluorescent imaging and localization of labeled MSC-EVs in retinal flat mounts: Exo-green MSC-EV-injected ischemic and normal rats were anesthetized at different time points (1, 3, and 7 days) after intravitreal injections and subjected to whole animal perfusion-fixation with PBS and 4% paraformaldehyde. Following enucleation, the eye cups were prepared by removing the cornea, lens and vitreous. The eyecups were post-fixed in 4% PFA for 30 min, washed twice in PBS, and permeabilized with PBST (0.3% Triton X-100 in PBS, twice). The eye cups were blocked overnight in 2% Triton X-100, 10% normal serum and 1 mg/ml BSA. The primary antibodies anti-IBA-1 for retinal microglia (1:500, Novus Bio, Littleton, Colo.), and anti-Brn-3a for retinal ganglion cells (1:500, EMD Millipore), and anti-β-tubulin III for retinal neurons (1:500, Sigma), were incubated with the eyecups at 4° C. for 48 h followed by washing and incubation with the appropriate secondary antibodies (Alexa Fluor 555 and 647, Molecular Probes, Thermo-Fisher) for an additional 48 h at 4° C. The samples were washed again, and the retinal tissues carefully dissected from the choroid and placed on a glass slide and mounted with Pro-Long Diamond Antifade Mounting Solution with DAPI (Life Technologies, Thermo-Fisher). Slides were imaged using a Zeiss 710 confocal at 63 and 100× oil immersion magnification, and images deconvoluted using Zeiss Zen v2.4 software.

Fluorescent TUNEL: Fluorescent TUNEL (terminal deoxynucleotidyl transferase-mediated dUTP nick end labeling assay) was performed with Apop Tag Red In Situ Apoptosis Detection Kit (Millipore-Sigma) on 7 μm thick cryosections at 24 h post-MSC-EV injection (48 h after ischemia). This is consistent with the time course of apoptosis that was previously described in retinal ischemia, where peak TUNEL was present 48 h after ischemia. Briefly, cryosections were fixed and hydrated in 4% paraformaldehyde followed by ethanol: acetic acid (2:1) post fixation. Sections were then exposed to equilibration buffer and incubated in TdT enzyme for 1 h in a humidified chamber followed by application of anti-digoxigenin conjugate for 30 min at room temperature, with the slides covered to protect them from light exposure. Sections were mounted using Prolong Diamond Antifade Mounting Agent containing DAPI.

Imaging was performed at 20× magnification on a Zeiss Axiovert 100 inverted microscope using Metamorph 7.3. The images were processed and analyzed using ImageJ. In brief, the inner and outer nuclear retinal cell counts for DAPI (total cell nuclei), and the TUNEL stained nuclei were counted using an automated cell counting macro in ImageJ, utilizing the Cy3 channel. The TUNEL cells of the retinal ganglion cell (RGC), inner nuclear, and outer nuclear layers were counted blindly without knowledge of the group name.

### MSC-EV Vitreous Humor Binding Assay:

The vitreous humor was extracted from normal rat eyes. After measuring the protein concentration, dilution to 50 μg/100 μl was performed in coating buffer (0.2M sodium bicarbonate, pH 9.4) and 96 well plates were coated with the vitreous proteins overnight at 4° C. Plates were washed and incubated for 1 h at room temperature with increasing amounts of fluorescently labeled MSC-EVs. Fluorescence from the bound MSC-EVs after washing was measured using a BioTek ELISA plate reader with the appropriate band pass filter sets and the results were plotted against MSC-EV amount to obtain the binding curves.

### Statistical Analysis:

Data were expressed as mean±standard deviation (SD), and compared by ANOVA where appropriate, and by t-testing. Analyses were performed using Stata version 10.0 (College Station, Tex.).

### Results

### Characterization of MSC-EVs:

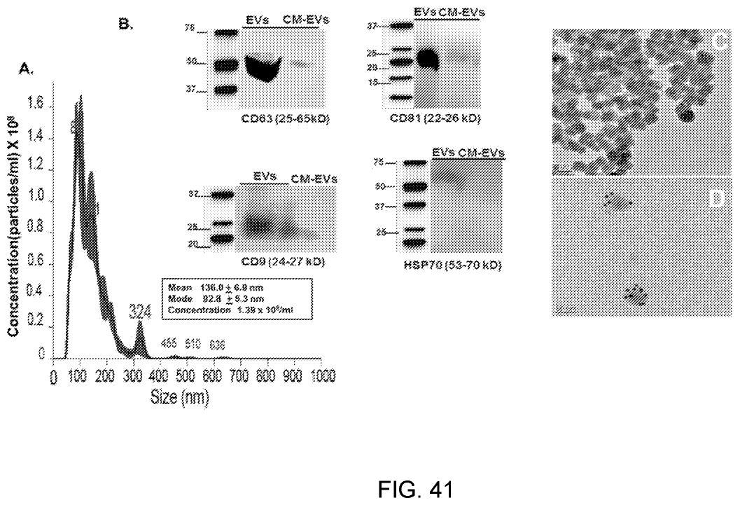

The purified MSC-EVs were characterized by NTA, immunoblotting, and EM. EVs are a complex mixture of membrane-bound vesicles released from most cells, and according to their size they have been classified as microvesicles (100-800 nm), exosomes (50-150 nm), and the much larger apoptotic bodies. MSC-EVs were found to be exosomal in their size and properties. Analysis of size and concentration of isolated EVs using NTA demonstrated a bell-shaped curve with the majority of the area under the curve falling within the characteristic exosomal size range of 50-150 nm, a peak at 89 nm, and a modal size of 93 nm. Another peak at 141 nm likely represents a mixture of exosomes and microvesicles, and the smaller peak at 324, the less abundant microvesicles (FIG. 41A). Western blot demonstrated exosome surface markers CD81, CD63, CD9, and HSP70α in the exosomal lysates, and not in exosome-depleted conditioned medium (FIG. 41B). The exosomal lysates were probed for tubulin as negative control for intracellular protein and no positive staining was observed (data not shown). TEM (FIG. 41C) showed particle shape and diameter of approximately 100 nm consistent with exosomes, and immuno-gold EM labeling for CD63 (FIG. 41D) showed the presence of CD63 on the exosome surface, confirming the immunoblotting results and that exosomes constitute most of the MSC-EVs in agreement with other studies.

MSC-EVs are Endocytosed by R28 Retinal Cells Via Specific Mechanisms:

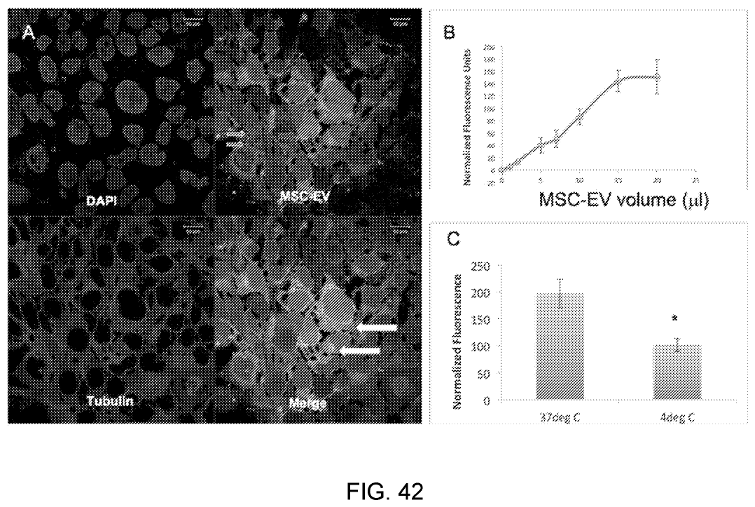

These experiments were performed to identify the basic mechanisms that control MSC-EV internalization by retinal cells. It was first confirmed that MSC-EVs are endocytosed by R28 retinal cells. FIG. 42A is a representative confocal image demonstrating that fluorescently labeled MSC-EVs were endocytosed by R28 cells in culture. Most of the R28 cells contained MSC-EVs indicating a high uptake efficiency. The MSC-EVs were visualized as punctate staining as well as agglomerates within the cells and across the nuclei. Yellow or orange staining in the composite image (lower right panel of FIG. 42A) indicated overlap with tubulin, showing that MSC-EVs were in the cytoplasm. FIG. 42B shows dose-dependent, saturable endocytosis of fluorescently labeled MSC-EVs. Furthermore, endocytosis was reduced significantly at 4° C., indicating temperature dependence (FIG. 42C). Taken together, these results indicate the presence of a controlled, energy-dependent endocytic mechanism for MSC-EVs in retinal cells.

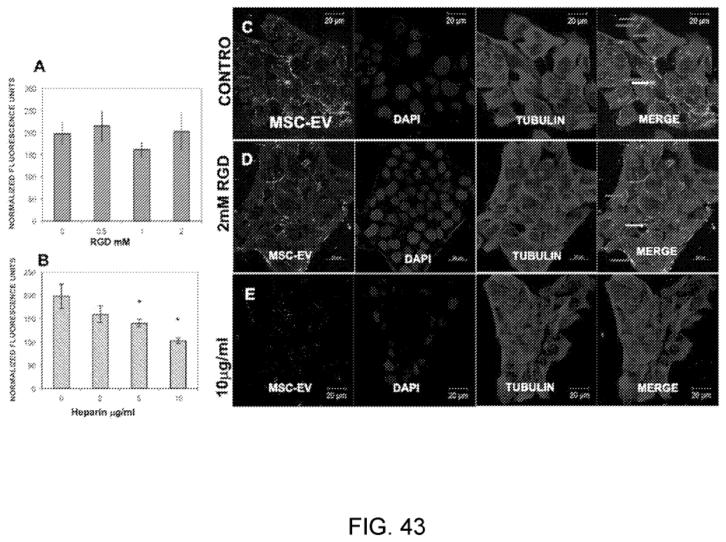

Next the study aimed to identify the endocytic receptors. Studies have shown involvement of integrins in EV endocytosis in some cell types. To analyze integrin involvement in the endocytosis of MSC-EVs by R28 retinal cells, integrins on the R28 cell membrane were blocked by pre-treatment with increasing concentrations of the integrin-binding Arginyl-glycyl-aspartic acid (RGD) peptide. No statistically significant impact upon MSC-EV endocytosis was observed (FIG. 43A). Conversely, when MSC-EVs were pre-treated with heparin to mimic the binding to HSPGs on the R28 plasma membrane, EV endocytosis was significantly and dose-dependently blocked (FIG. 43B). Confocal microscopy qualitatively confirmed these quantitative results (FIGS. 43C-E). The results ruled out integrin involvement in the endocytosis of MSC-EVs and indicated a role for cell surface HSPGs.

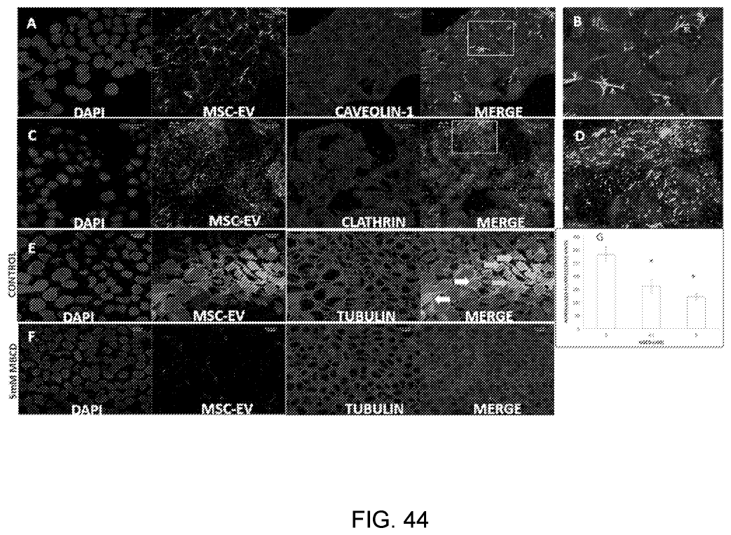

Depending on the receptors involved and the type of ligand, endocytosis can occur via a clathrin- or caveolin-mediated process. Endocytosed MSC-EVs were analyzed by confocal microscopy for co-localization with caveolin-1 (a marker for caveolae and lipid rafts) and clathrin (which forms clathrin-coated endocytic pits). Representative confocal images (FIGS. 44A-B) show co-localization of the endocytosed MSC-EVs with caveolin-1. No co-localization was observed with clathrin in FIGS. 44C-D. Blocking caveolar-mediated endocytosis by MBCD, to disrupt membrane cholesterol, dose-dependently inhibited MSC-EV endocytosis (FIGS. 44E-F, and FIG. 44G).

### MSC-EVs Attenuate Cell Death in R28 Cells Subjected to OGD In Vitro:

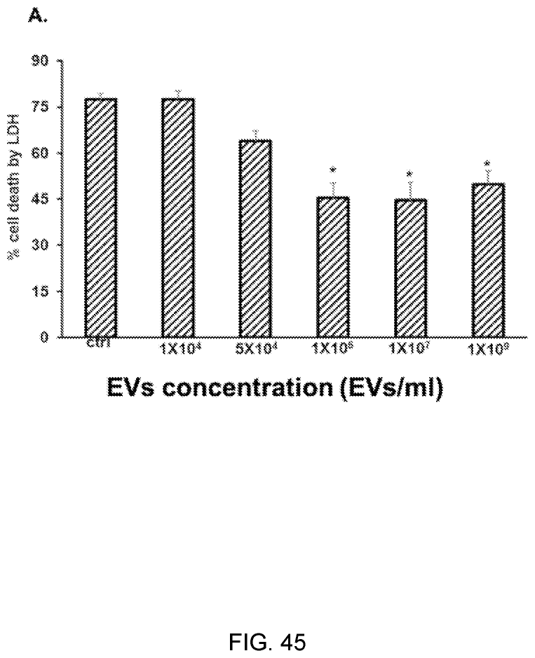

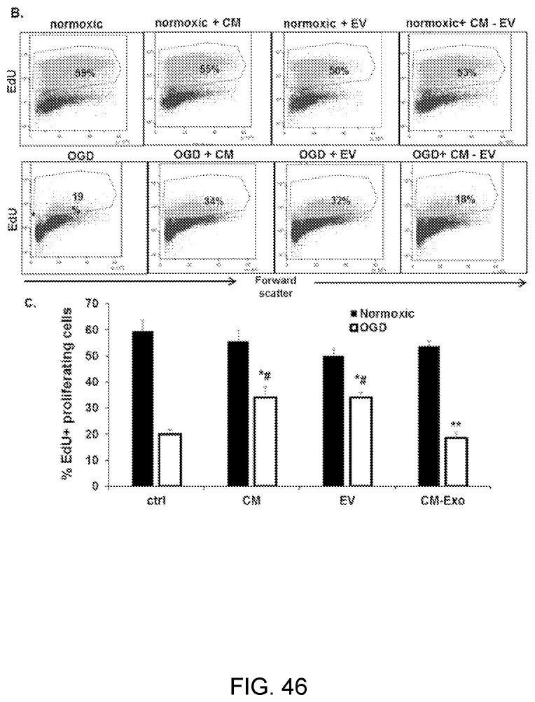

Oxygen glucose deprivation (OGD) results in cell death and mimics ischemic conditions in vitro. The hypothesis that MSC-EVs rescue R28 cells from OGD-mediated cell death was tested. R28 cells pre-treated for 24 h with or without varying doses of MSC-EVs were subjected to OGD. FIG. 45 shows that in the absence of MSC-EVs, OGD induced cytotoxicity was >75%. Cytotoxicity was significantly reduced in a dose-dependent and saturable fashion with MSC-EV pre-treatment. To evaluate the effect of MSC-EVs on the proliferative state of R28 cells, flow cytometry analysis was performed for EdU positive cells (FIG. 46) under both normoxic and OGD conditions. Under normoxic conditions, the percentage of EdU positive cells was no different between PBS control, MSC conditioned medium, EVs, or EV depleted conditioned medium. A slight decrease in the percentage of proliferating cells was observed with the EVs although this change was not significant. Conditioned medium as well as EVs significantly improved the number of proliferating cells under OGD conditions. When conditioned medium depleted of EVs was used, the protective effect was abrogated suggesting that the protective effect is likely due to EVs in the conditioned medium.

### MSC-EV Administration Following Retinal Ischemia In Vivo Attenuates Ischemic Damage:

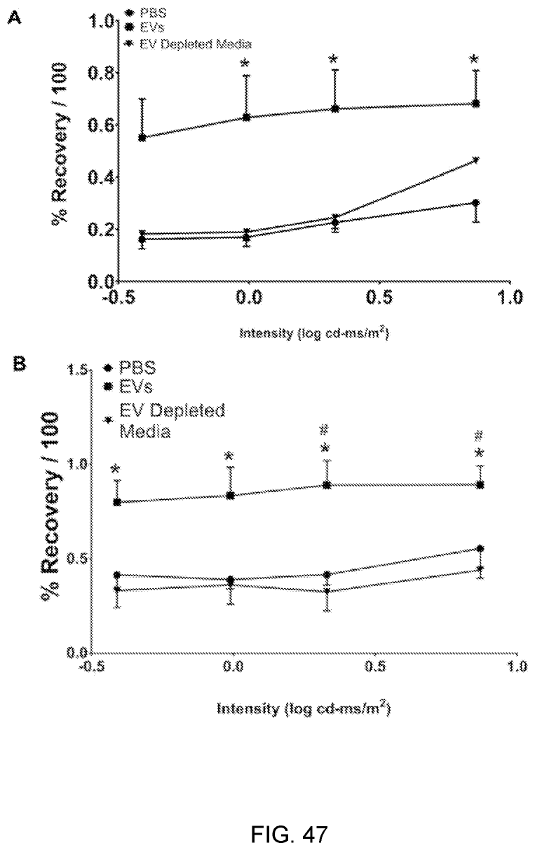

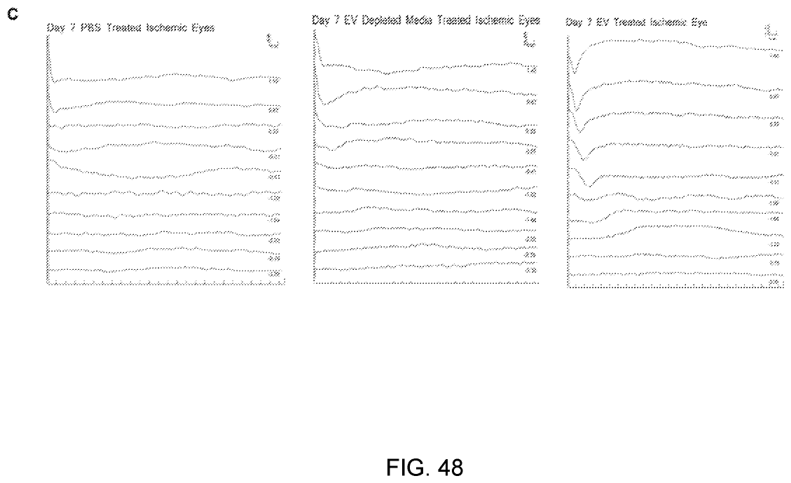

We tested the hypothesis that MSC-EVs reverse the effects of ischemic injury in vivo in a rodent model. MSC-EVs injected intra-vitreally 24 h after ischemia significantly improved the recovery of the a- and b-wave amplitudes of the ERG in comparison to both PBS vehicle and EV-depleted conditioned medium from MSCs (FIG. 47). Electroretinogram (ERG) results were normalized to control eyes and to the baseline prior to ischemia which accounts for day-to-day variation in the amplitudes of the non-ischemic eyes. Y axis is % recovery relative to baseline/100 and x-axis is stimulus intensity in log cd-ms/m². The amplitudes are shown as mean±SD. There was significant improvement of recovery of the a-wave amplitude with intravitreal MSC-EVs vs PBS control, and significant improvement of recovery of the b-wave amplitude with intravitreal MSC-EVs compared to PBS and MSC-EV-depleted medium controls.

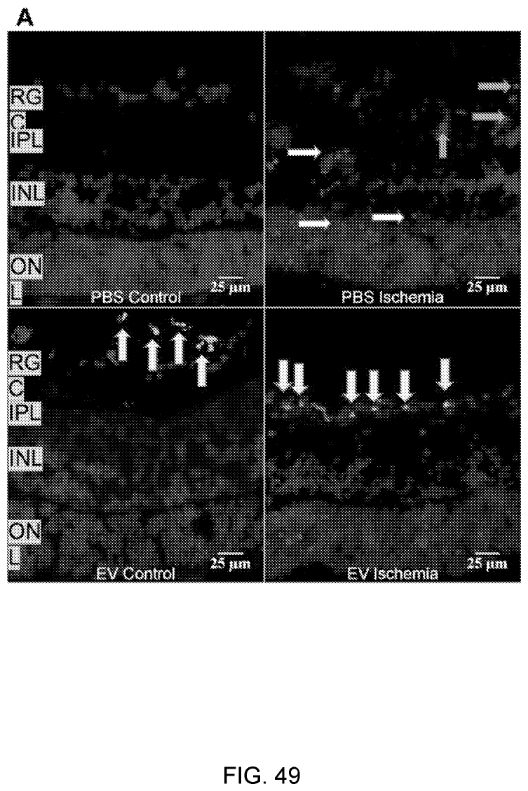

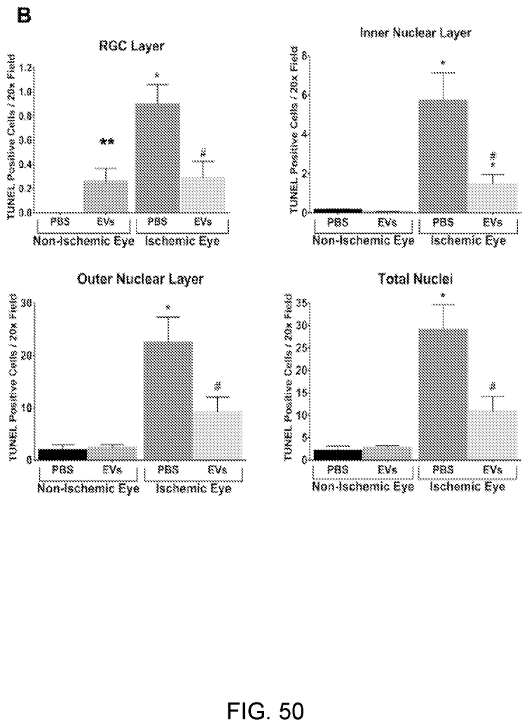

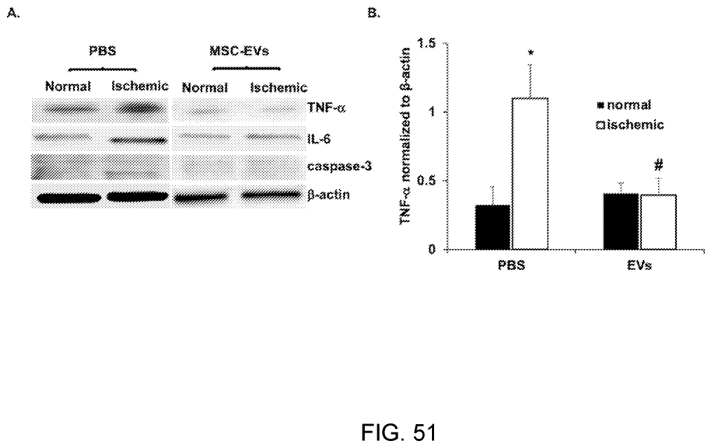

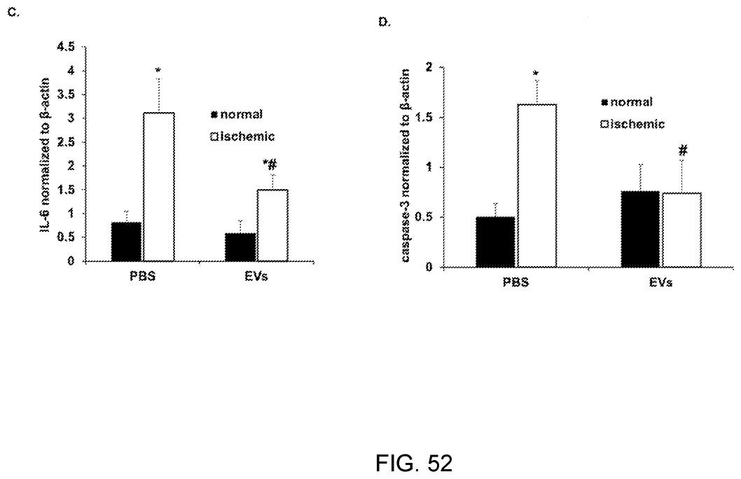

The significant improvement of the a- and b-waves is also evident in the representative ERG stimulus-intensity traces shown in FIG. 48. To evaluate if the MSC-EV functionality was related to its anti-apoptotic effects, fluorescent TUNEL was quantitated on retinal cryosections (FIGS. 49 and 50). MSC-EV injection 24 h after ischemia significantly reduced TUNEL in the inner and outer nuclear layers and in the retinal ganglion cell layers. There was an increase in TUNEL in the RGC, but not in other cell layers in MSC-EV-injected non-ischemic retinae. In whole retinal homogenates, levels of the inflammatory mediumtors TNF-α and IL-6 were significantly reduced upon MSC-EV treatment following ischemic injury vs vehicle controls (FIGS. 51A and 52C). There was also a significant reduction in cleaved caspase-3 levels in MSC-EV treated ischemic retina vs vehicle controls (FIGS. 51A and 52D). In non-ischemic MSC-EV injected retinae, there was no significant change in levels of any of the three proteins, although cleaved caspase 3 was observed with EV or PBS treatment under non-ischemic conditions, there was no statistically significant increase with respect to PBS control. Taken together, the results in FIGS. 47-52 indicate that MSC-EV treatment following retinal ischemia leads to functional improvement in the retina via reduction of apoptosis and neuro-inflammation. In addition, the results indicate, that with the exception of an increase in TUNEL in the RGC layer, no evidence of inflammation or apoptosis triggered by EVs in normal retina was detected by the measurement techniques.

### MSC-EV Uptake and Distribution in Retina In Vivo:

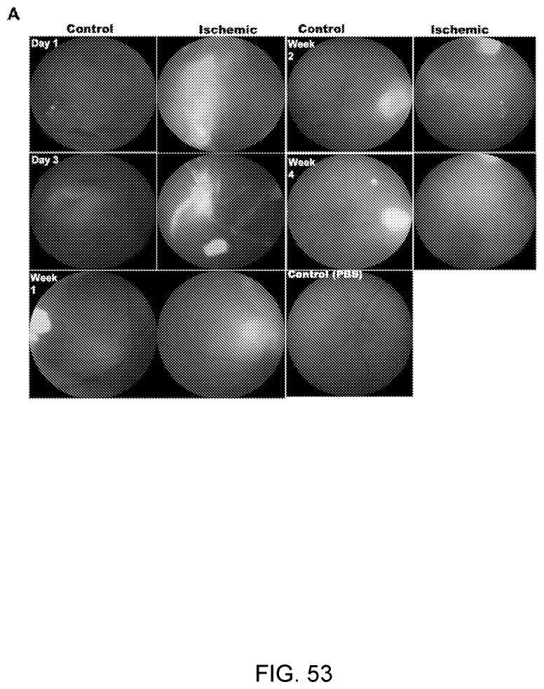

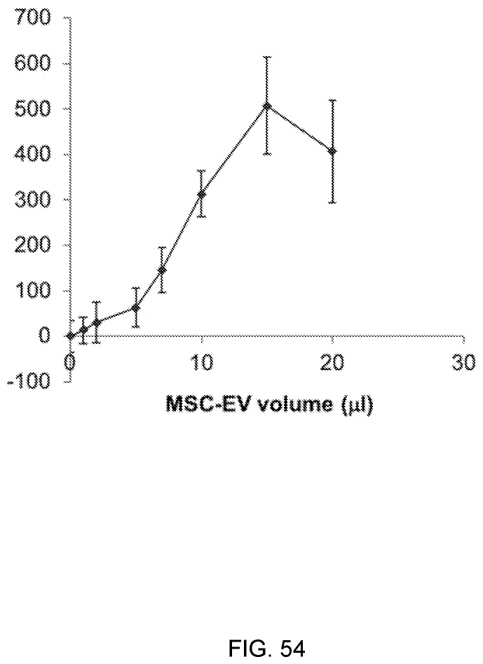

Having observed the functionally neuroprotective effects of MSC-EVs in the ischemic retina, the distribution of the EVs after injection into the vitreous humor was evaluated. FIG. 53 displays localization of labeled MSC-EVs in the vitreous humor and retina. There was persistent retention of MSC-EVs in vitreous humor up to 4 weeks after intra-vitreal injection. Ischemic retina demonstrated increased MSC-EV uptake vs control non-ischemic eyes. In addition, large deposits of accumulated MSC-EVs in the control and ischemic retina were observed. These results are entirely explainable as fluorescence from the MSC-EVs, as previously it has been shown that fluorescein injected into the vitreous humor is cleared within 48 h. To test the vitreous humor's capacity as a reservoir for MSC-EVs, quantitative binding experiments were performed on assay plates coated with protein isolate from the vitreous humor and increasing dose of fluorescently labeled MSC-EVs. Results presented in FIG. 54 illustrates dose-dependent and saturable binding of MSC-EVs to vitreous humor proteins. This result explained the presence of accumulated MSC-EVs in the vitreous humor.

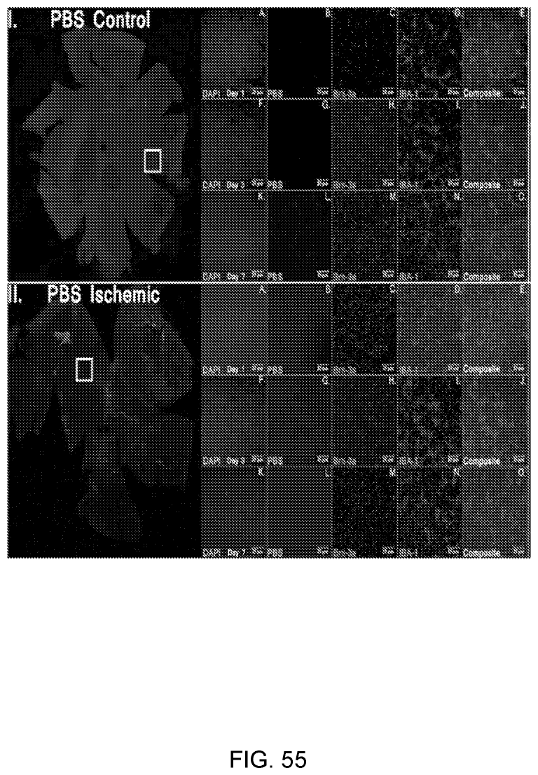

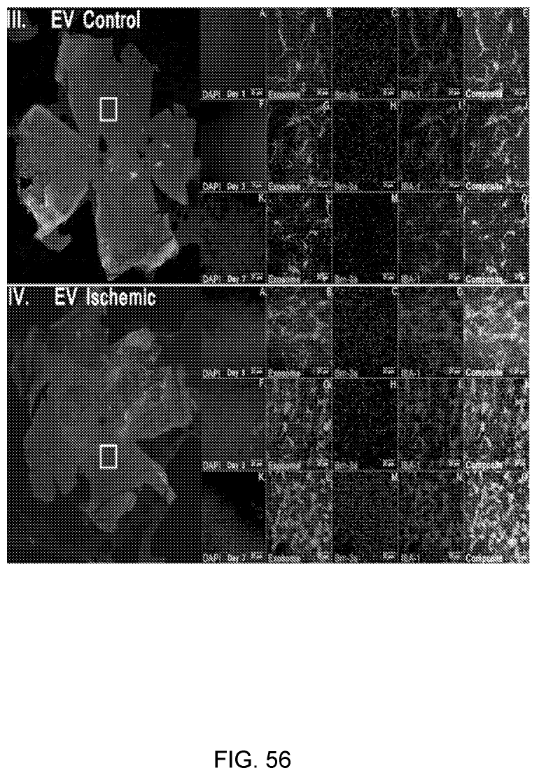

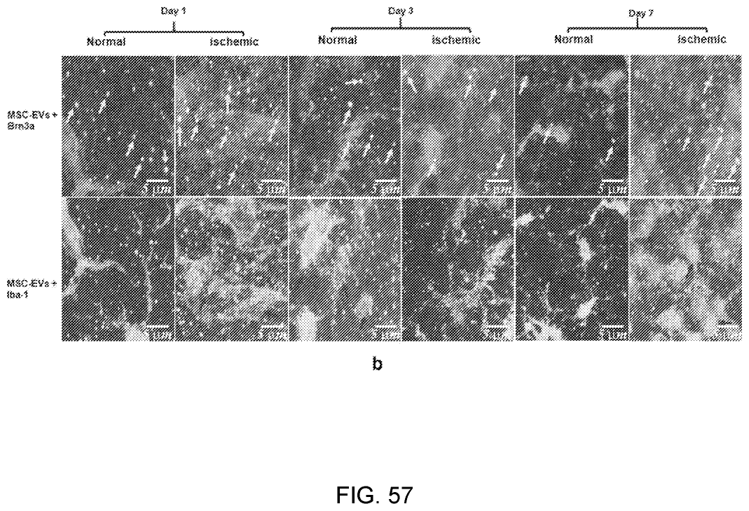

To evaluate if MSC-EVs are preferentially endocytosed by specific retinal cells in vivo, retinal flat mounts prepared at different time intervals after fluorescently labeled MSC-EV injections were immuno-stained with markers for different retinal cells. Brn3A was used as the marker for RGCs and IBA-1 for retinal microglial cells. Flat mounts (FIGS. 55 and 56) showed distribution throughout the retina and persistence of MSC-EVs at one week after injection (time points later were not evaluated). FIG. 57 shows that both RGCs and microglia take up MSC-EVs. Moreover, FIGS. 55-57 qualitatively show greater microglial amoeboid, or activated morphology in ischemic, non-MSC-EV treated retinae vs MSC-EV-treated retinae, suggesting reduced microglial activation in ischemia in the presence of MSC-EVs.

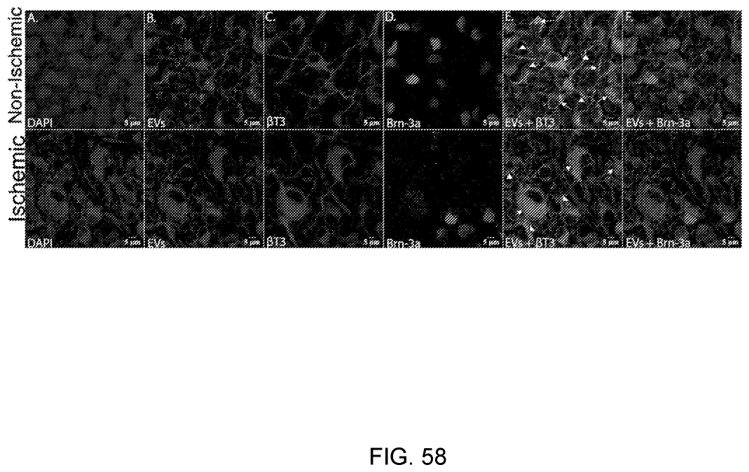

FIG. 58 contains 100× confocal microscopic images of retinal flat mounts from non-ischemic (upper panels) and ischemic retinae (lower panels) respectively from retinal tissues harvested 24 h after administration of MSC-EVs, that corresponds to 48 h after ischemia. MSC-EVs are present in retinal neurons, as indicated by presence of MSC-EVs in cells labeled with specific neuronal marker β-tubulin Ill (FIG. 58E) as well as in axonal or dendritic projections (arrows in FIG. 58E) and in Brn3a-positve cells (FIG. 58F) indicating that the MSC-EVs are endocytosed by the retinal neurons and by RGCs.

This study presents new data on the neuroprotective effects of MSC derived EVs in the retina that are relevant to treatment of ischemia-related retinal degeneration, as well as to the treatment of neuronal injuries in general. The vesicular populations were referred to as MSC-EVs. Although the definition of exosomes is evolving, a modal size of 93 nm along with the expression of exosome specific markers indicate that the population is predominantly exosomes as defined by Kowal et al. These studies using MSC-EVs depict a consistent progression from stem cell-based therapy to cell-free therapy for retinal tissue neuroprotection and regeneration and regenerative medicine in general. As cell-free therapy, MSC-EVs offer a safe, biomimetic alternative with lower oncogenic and immunological risks and greater target specificity. To date, only a small number of studies have examined EV therapy in the retina, one demonstrating therapeutic effect in an optic crush model and the other in a glaucoma model, both in rats. However, no prior studies have examined the mechanisms of uptake of EVs in retina, their vitreous humor and cellular distribution, nor effects upon ischemic insult.

These results indicate that EVs are endocytosed by retinal R28 cells in a dose-dependent, saturable, and temperature-dependent manner, suggesting the involvement of a receptor-mediated endocytic mechanism. Published studies show that EVs from different sources undergo endocytosis via different mechanisms owing to a change in the composition of the EV membrane. The clathrin and caveolar pathways, phagocytosis, and macro-pinocytosis have all been implicated in endocytosis of EVs. These results indicated that MSC-EVs are endocytosed by R28 retinal cells via the caveolar endocytic pathway mediated by cell surface HSPG receptors. Quantitative studies showed that MSC-EV endocytosis by R28 cells was dose-dependently blocked by disrupting the cell membrane cholesterol or by competitively blocking HSPG binding sites on the EVs with heparin. Furthermore, confocal microscopy revealed that membrane bound and endocytosed EVs co-localize with caveolin-1, further confirming the role of the caveolar endocytic process. Considering that the caveolar pathway routes its cargo away from lysosomal degradation and considering the functional activity of the endocytosed EVs, it is possible that for the EVs to be functionally active, this mode of endocytosis is ideal.

These mechanistic studies highlight the potential of MSC-EVs as biomimetic agents for treatment of neurodegenerative diseases and nerve injuries in general. From a therapeutic perspective, the effectiveness of MSC-EVs is dependent on the efficiency of endocytosis by target cells. Improved endocytic efficiency can promote greater target-specificity at the site and reduce ectopic effects. Therefore, the results outlining the endocytic mechanism open up avenues for future studies that can be aimed at engineering EVs for enhanced delivery by targeting these endocytic pathways. In addition, they can also serve as quality control points for function-specific engineered exosomes to ensure that intrinsic endocytic processes are not altered upon generation of engineered EVs. However, the R28 cell line is an immortalized retinal cell line that displays both neuronal and glial cell properties. While the ability of these cells to proliferate enables measurement of a critical cell function, further studies using primary retinal cells can be required to confirm the endocytic mechanism identified here.

In the in vitro OGD model, results indicated a dose response effect of MSC-EVs and saturation that corroborates well with the endocytosis data. Furthermore, cytotoxicity studies using quantitation of proliferative cells via flow cytometry revealed that MSC-EVs rescue the R28 cells from OGD insult. Taken together, these studies showed that MSC-EVs have the potential to promote the survival and proliferation of retinal neurons that have been subjected to ischemia-type stress in vitro. These results encouraged the evaluation of MSC-EVs post ischemic insult in vivo in a rodent model.

The onset of retinal ischemic injury in vivo is manifested as neuronal cell death, apoptosis, and neuroinflammation resulting in RGC loss, blood-retinal barrier permeability, and neurodegeneration. Therapeutic effects of MSC derived EVs are reported in a wide range of inflammatory diseases including, but not limited to ischemia-reperfusion injury in brain, heart, kidney as well as in neurodegenerative diseases. The results demonstrate that MSC-EVs render their neuro-protective effect by decreasing neuroinflammation and neuronal apoptosis. These results provide an insight into the mechanism behind MSC-EV action in the retina under ischemic injury and serve as a foundational knowledge that can be used to generate engineered EVs with function-specific miRNA cargo with anti-inflammatory and anti-apoptotic properties.

Prior to the current study, the uptake, retention, turnover and prolonged effect of EVs in the retina have been addressed in only a limited manner. The role of the vitreous humor in EV retention and the subsequent endocytosis by different cells of the retina has remained unexplored. A few recent studies injected EVs in single dose, weekly, and monthly in a model of glaucoma, and reported enhanced protection with multiple administration, while another group reported high dose, single injection EV (15×10⁹ particles/ml) induced protection in experimental autoimmune uveitis. Dose dependence and toxicity studies using MSC EVs with retinal or neuronal cells under normal and ischemic conditions have not been performed. The results using concentrated EV injections in an in vivo model did not cause any deleterious effect in the ERG functional studies, nor increased inflammatory mediators. Additionally, the in vitro and in vivo results indicate a mild level of toxicity of MSC EVs under normoxic conditions albeit being statistically insignificant. However, there was no corresponding increase in the inflammatory markers or increase in cleaved caspase 3 in retinal homogenates in the normal non-ischemic retinae. Further studies will be required to evaluate if there is any functional effect of EVs specifically on the retinal ganglion and amacrine cells in the RGC layer.

Multiple dosing or higher doses are not required if EVs can traverse the vitreous and reach target cells in the inner retina after administration. Greater quantities of MSC-EVs were observed in ischemic compared to non-ischemic retinae. Additionally, they were more concentrated in RGCs and in microglial cells. This increase in the uptake of EVs by ischemic cells is potentially advantageous, but the mechanism of this effect requires further investigation. Preferential uptake by cells in ischemic neuronal and glial has potentially important implications in therapeutic development of EVs as biomimetic agents for treatment of nerve injuries and neuro degenerative diseases. Likewise, a surprising result was that while EVs robustly attenuated TUNEL throughout the retina and decreased cleaved caspase 3 presence indicating a decrease in apoptosis, the labeled EVs were not found deeper than the retinal ganglion cell layer at the time of peak apoptosis (48 h after ischemia). This suggests that the EV effects on apoptosis are either due to altered retinal cell-to-cell signaling, e.g., via Muller glial cells that traverse most of the retina, or are due to release of EV induced anti-apoptotic factors from the cells that have endocytosed them. It is also possible that with more time, the MSC-EVs penetrate more deeply into the retina, and further studies will be required to test this hypothesis.

We showed specific uptake of MSC-EVs in vivo by RGCs and microglia, as well as by retinal neurons. The targeting of RGCs by the EVs supports development of EVs and engineered EVs for the treatment of glaucoma and other diseases of optic nerve that result in degeneration of RGCs. These studies can also serve as a prelude to neuro-targeted EV therapy for treatment of specific nerve cells. It is interesting that MSC-EVs were present in axonal or dendritic projections from RGCs and retinal neurons. Further studies are necessary to determine if the MSC-EVs are transported along the axons, as this can enable novel access to the optic nerve via retrograde transport. Microglial activation was not quantitated in this study but decreased amoeboid formation after ischemia in MSC-EV-injected eyes suggests another potential target for MSC-EV-therapy. Microglial activation in the retina can be a pathogenic factor in various diseases including diabetic retinopathy, glaucoma, and age-related macular degeneration, thus MSC-EVs targeting microglia could be a novel treatment modality.

This study indicates that intravitreal injection produced uptake of MSC-EVs uniformly in the retina (as seen in the distribution on flat mounting). The MSC-EVs remained in the vitreous humor for up to 4 weeks after injection and quantitative binding experiments to vitreous humor-derived proteins suggest that the effect is due to binding to vitreous humor proteins in a dose-dependent and saturable manner. As a result, the vitreous humor serves as a reservoir for release of EVs into the retina and this property could be used advantageously to prolong EVs effects and minimize the number of injections necessary to produce long-term effects.

One of the significant challenges associated with the use of EVs for therapeutic purposes is the ability to deliver them site specifically to relevant tissues. From this perspective, the identification of EV binding kinetics to vitreous proteins is valuable data for the biomaterials community. Future studies aimed at identifying peptide sequences present in vitreous collagens can be used to generate engineered biomimetic matrices used to deliver EVs and possibly promote controlled release of EVs to aid repair and regeneration of not just neuronal tissues, but other tissues as well.

MSC-EVs are endocytosed by retinal cells in a receptor-mediated, dose-dependent and saturable manner. The endocytosed EVs can protect retinal cells from cell death in simulated ischemic conditions in vitro and in retinal ischemia in vivo. The findings on the involvement of HSPGs on the target cell surface in EV endocytosis and the binding of EVs to the vitreous serve as a basis for development of engineered EVs targeting these mechanisms for enhanced delivery and/or functionality. Furthermore, if these results can be extrapolated to other neuronal systems a common modality and a pathway for biomaterial based site-directed EV therapy can be established.

### Example 5: miRNA Reading in Various Engineered Exosome Populations

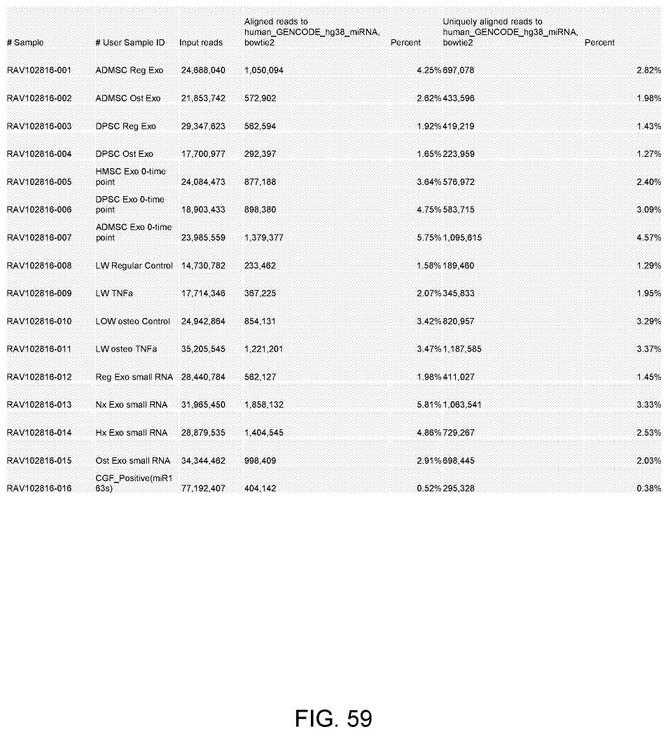

The raw reads of RNA-seq were mapped to miRNA reference genome GENCODE hg38 (only containing the miRNA sequences from GENCODE hg38). Several mapping software and different parameter settings were compared: the bowtie2 was determined to have provided the best mapping result. The identified miRNAs were differentially represented in exosomes, depending on how their parent cells were engineered (FIG. 59). In each sample, the number of reads for each miRNA were normalized by the library size (number of the total reads in the library). The top candidates, i.e. those with normalized reads above 100 in at least one exosome population, are shown in FIG. 60.

Because there were reads that could not be mapped to the miRNA reference genome, the conclusion was reached that the sample contains a large amount of non-miRNA sequences, which could be pi RNA or other long non-coding RNA.

### Example 6: Preparation of Hydrogel Exosome Composition

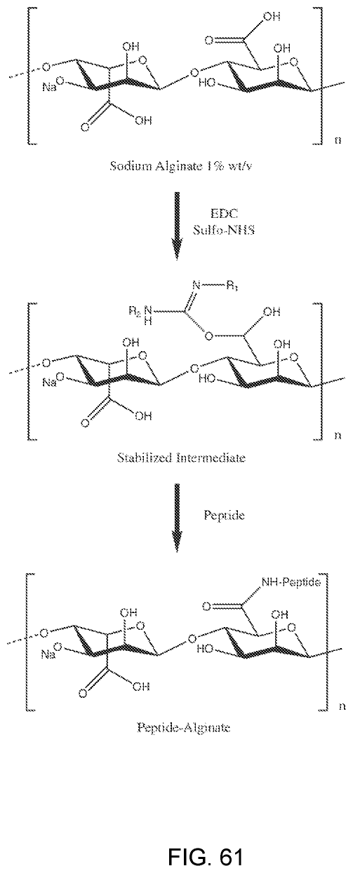

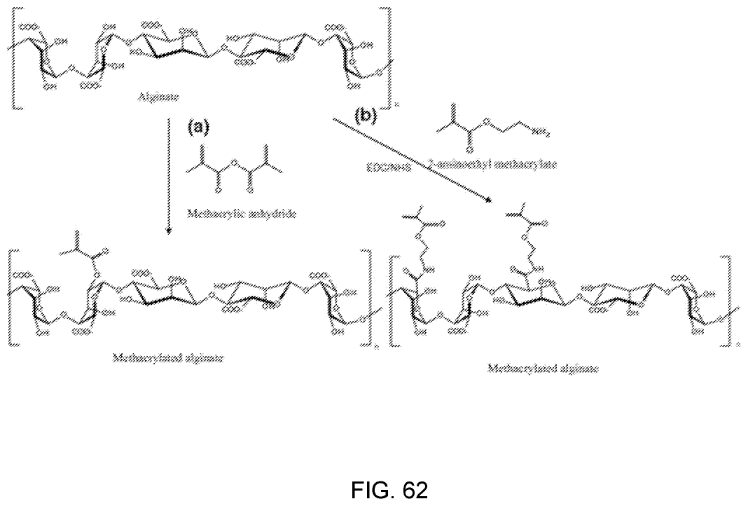

Methacrylic alginate with RGD/DGEA/GFPGER peptide modification was prepared as illustrated in FIGS. 61 and 62. In short, first native alginate powder (3 g) was dissolved in 300 mL of MES buffer (0.1 M MES, 0.3 M NaCl, pH 6.5) at 1% w/v. The solution was stirred until alginate was fully dissolved. Then, 1-ethyl-3-(3-dimethylaminopropyl)carbodiimide (150 mg; EDC) and N-hydroxysulfosuccinimide (84 mg; sulfo-NHS) were added into the solution, and the solution was stirred for 15 minutes. Then, the peptide powder (22.14 mg of GGGGRGDY (SEQ ID NO:9) or 23.46 mg GGGGDGEAY (SEQ ID NO:10) or GFPGER (SEQ ID NO:11)) to yield a 10 μmole/g alginate concentration and the solution was gently stirred for another 24 hours at room temperature. The solution was dialyzed against distilled H₂O for 7 days and lyophilized until dried. FIGURE

Next, the lyophilized peptide-conjugated alginate was dissolved in water at 2.5% w/v, and this solution was treated with 120 mL of methacrylic anhydride. The solution was adjusted and maintained at pH at 7 to 8 for 72 hours using 10 N NaOH solution. The solution was stirred for an additional 1-2 days until it solidified, and then water and 6 N HCl were added to dissolve the solid. The dissolved solution was poured into 600 mL of 100% alcohol, and the alginate precipitated. The precipitate was then dissolved in 120 mL of water, centrifuged, and washed again as needed. The methacrylate- and peptide-conjugated alginate was left to air dry.

Finally, 1-8 wt % of methacrylate- and peptide-conjugated alginate was mixed with exosome suspension (1×10⁶-1×10¹² exosomes of the disclosure) and polymerized by exposure to UV light to obtain hydrogel comprising the exosomes of the disclosure.

Schematics of this process are shown in FIGS. 61 and 62.

### Example 7: Evaluation of Hydrogel Exosome Composition

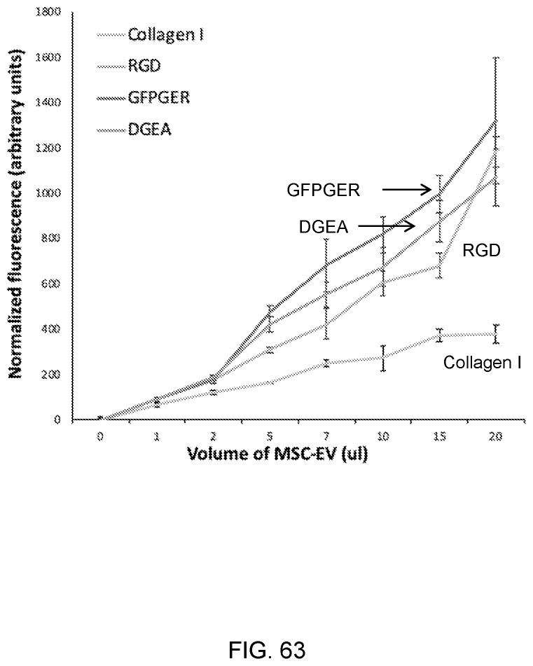

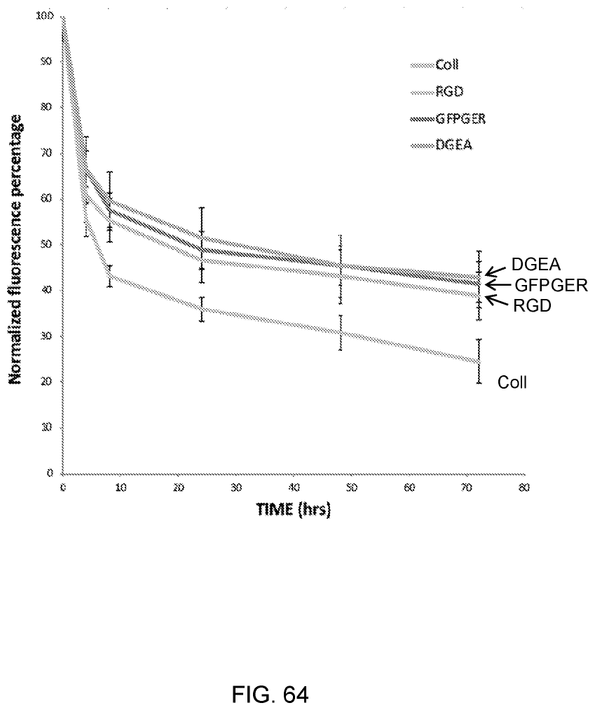

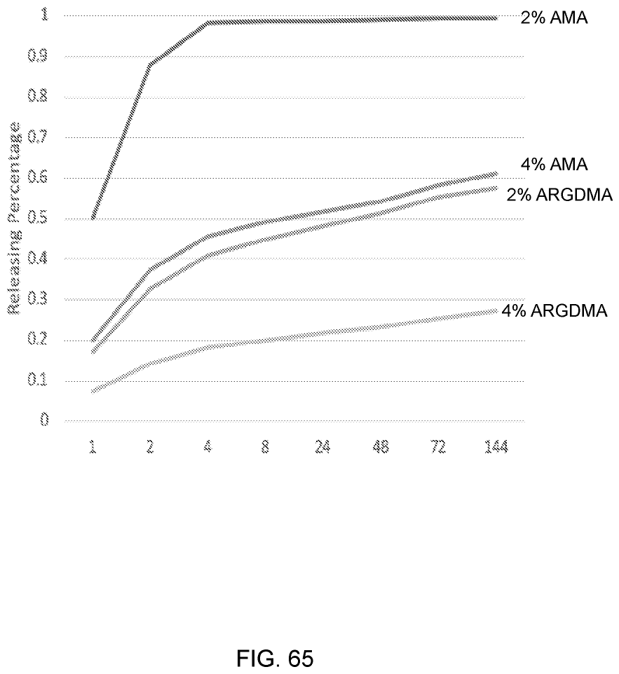

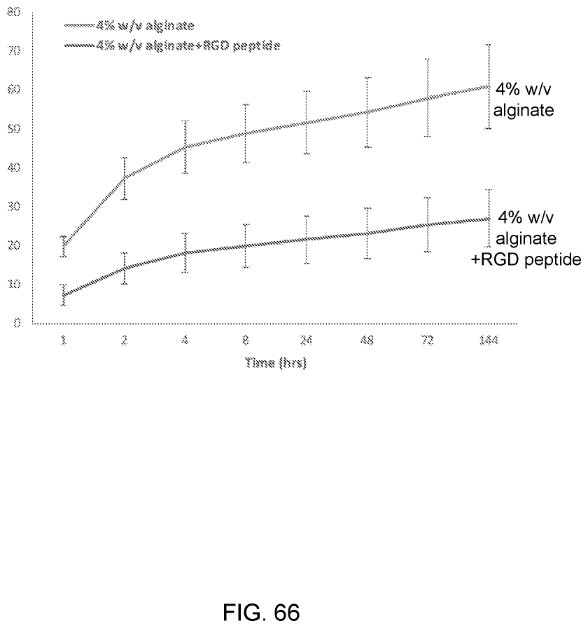

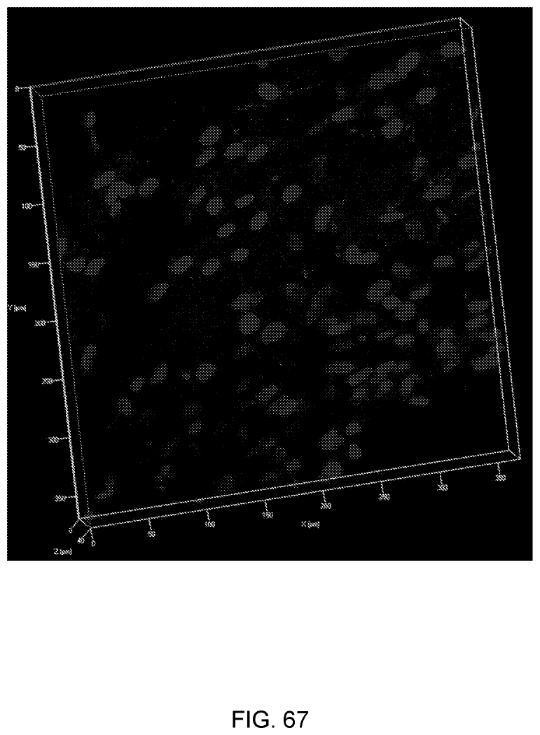

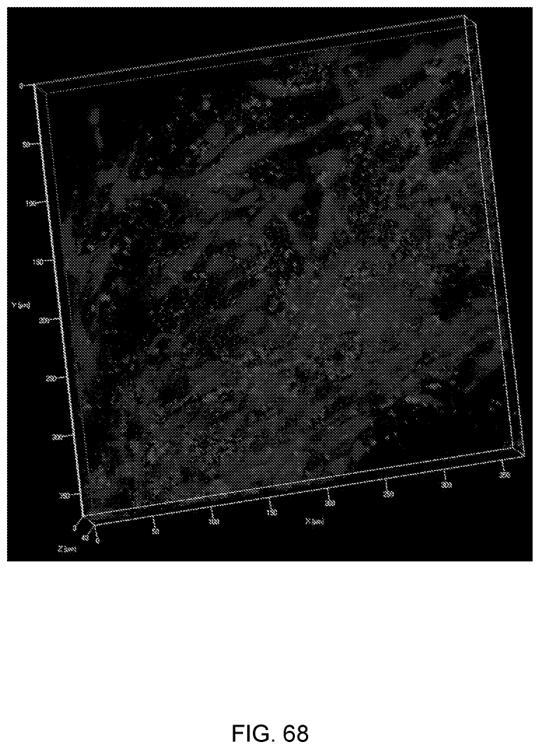

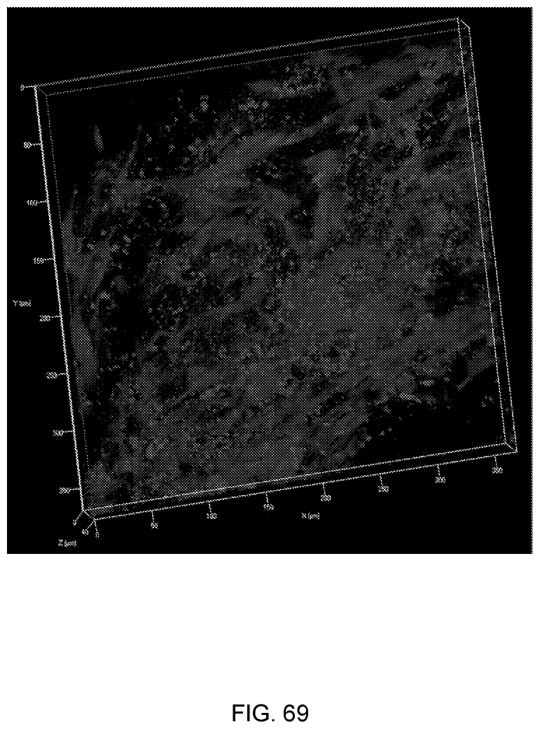

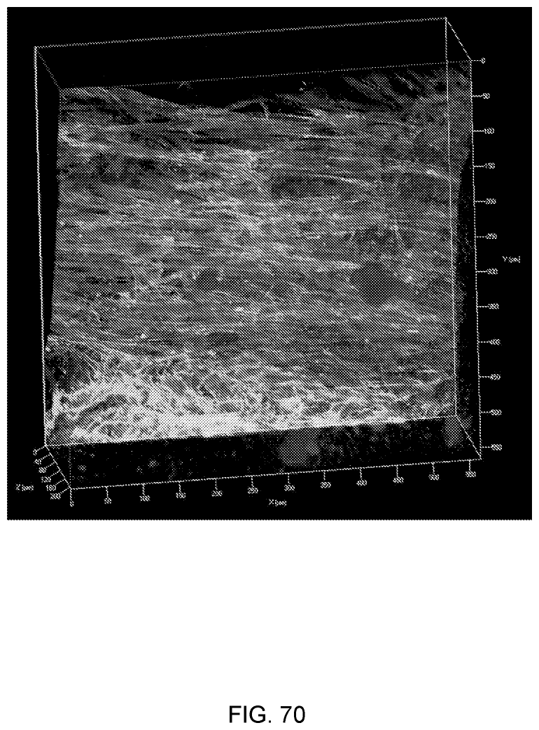

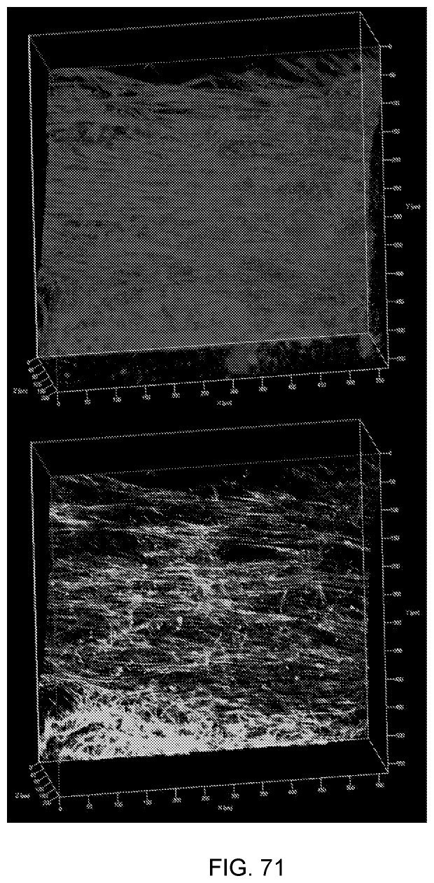

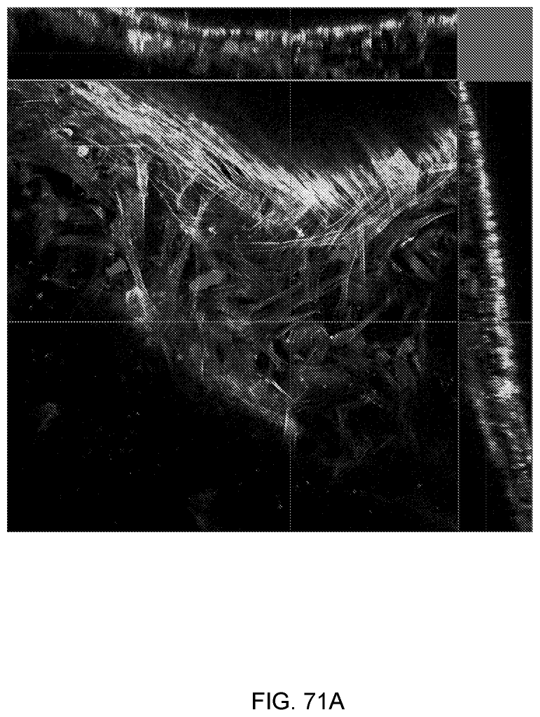

Exosome binding peptides that are representative derivatives of type I collagen and fibronectin were coated onto 96 well plates and the dose-depending binding of fluorescently labeled MSC exosomes to these peptides was evaluated (FIG. 63). Results indicate that MSC exosomes can bind to these peptides and that such ECM binding derivative peptides may be used as carriers for exosomes. Consequently, the release profile of the bound exosomes was also evaluated over (FIG. 64). The ability of MSC exosomes to be bound to hydrogels containing these binding peptides and delivered was evaluated in vitro by encapsulating the exosomes in alginate hydrogels with and without the binding peptides at 2 and 4% w/v alginate concentrations. Here RGD peptide was used as a proof-of-principle example. The results of these studies are presented in FIGS. 65 and 66. Overall, the results indicate that MSC exosomes are released from the hydrogels within a few hours if there is no tethering peptide (RGD here). On the other hand, in the presence of the tethering peptide, the release was slower. Note the increased retention of exosomes with RGD. To test if the encapsulated exosomes were endocytosed by colonizing cells, naïve MSCs were seeded on to alginate RGD hydrogels loaded with exosomes (stained). Nuclei (blue) and exosomes (green) were stained and imaged (FIGS. 67, 68, 69). Similar loading was also tested 3 days after post cell seeding. Exosomes (green) and actin (red) were stained and imaged (FIGS. 70 and 71). Results indicated that the bound and encapsulated exosomes were endocytosed by the colonizing cells indicating that the exosomes maintained their ability to be taken up by cells even after 3D encapsulation using the tethering peptides.

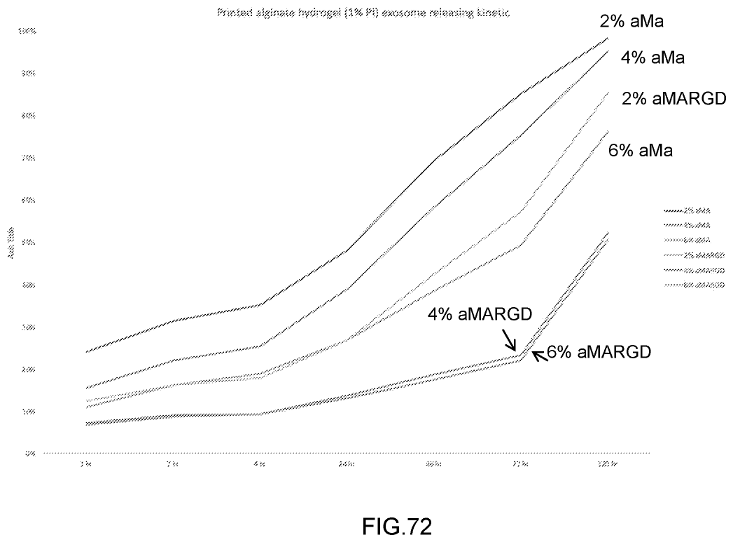

In one example, the hydrogels comprising the exosomes of the disclosure were formed using a 3D printing technique. These printed compositions were evaluated for exosome release kinetics, and the results are shown in FIG. 72.

### Example 8: In Vitro Experiments

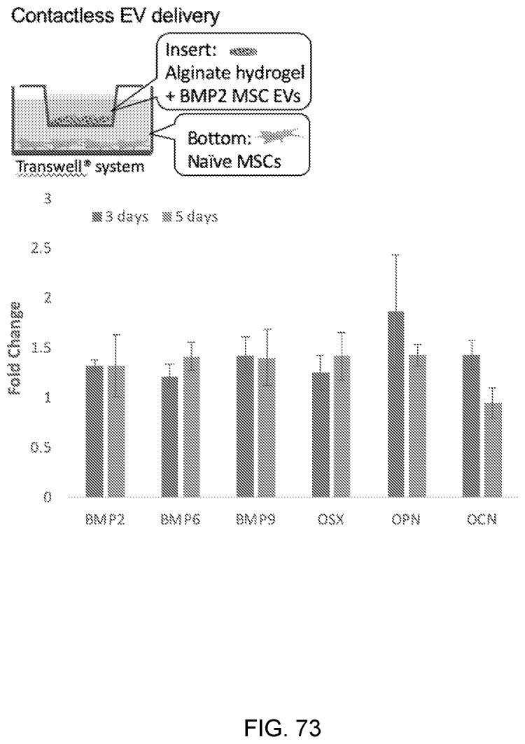

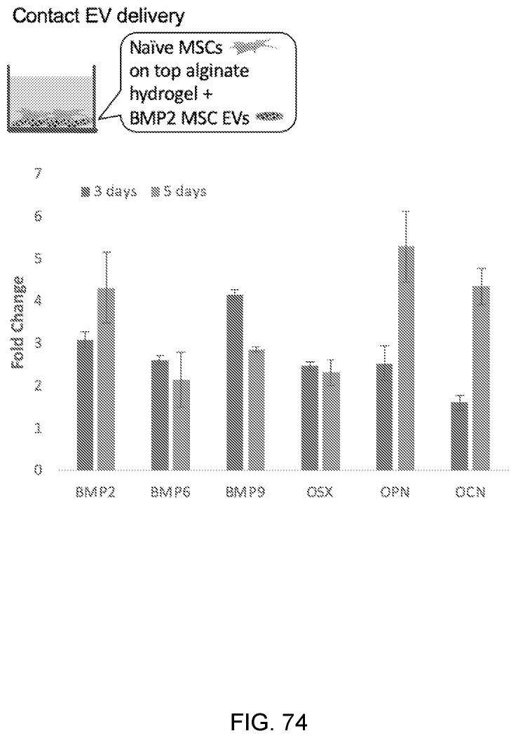

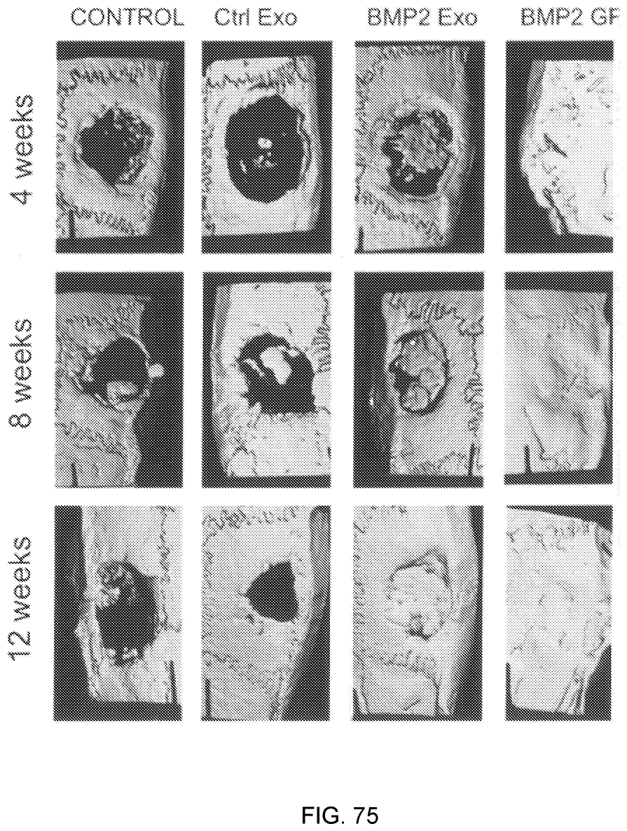

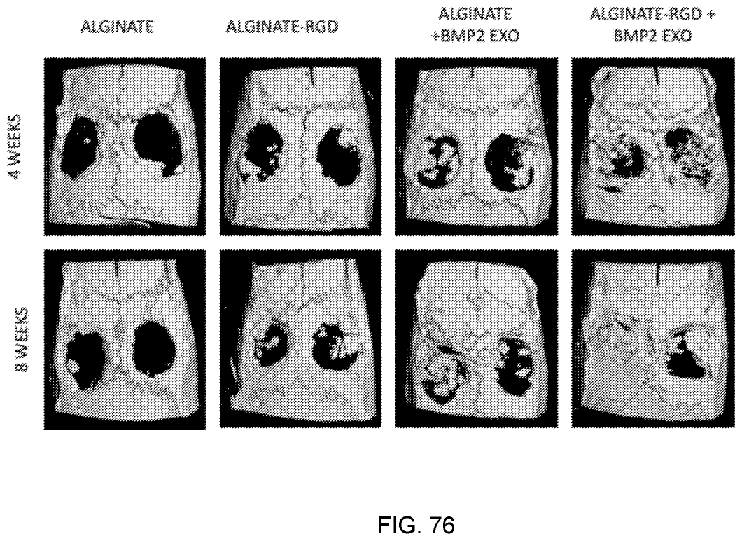

To test if the functionality of the exosomes are retained after encapsulation and release, Hydrogels containing BMP2 exosomes were tested via both a contactless (FIG. 73) and contact (FIG. 74) model. BMP2 exosomes were used here as there is extensive data on their ability to induce osteogenic differentiation in naïve stem cells in vitro and in vivo (FIGS. 75 and 76). Expression of various osteogenesis factors was tested. The experiment was designed to test the functional efficiency of the encapsulated and the released exosomes from the hydrogels. The results show that both the encapsulated and the released exosomes were endocytosed by cells and that the exosomes caused the intended change in the cells that took them up.

### Example 9: In Vivo Experiments

To evaluate the ability of engineered exosomes to regenerate tissues, BMP2 exosomes were tested on rats with calvarial defects: one on the right, and one on the left. Controls with no treatment and with non-engineered exosomes were used. Here, collagen sponges were used as exosome carriers. Bone regeneration was evaluated by μCT 4 weeks, 8 weeks, and 12 weeks. The most significant regeneration results were seen with the BMP2 exosomes. For a positive control, BMP2 growth factor was used. Results are shown in FIG. 75.

To evaluate if binding peptide carrying hydrogels can be used to deliver exosomes, Alginate RGD hydrogels and control alginate hydrogels containing BMP2 exosomes were tested on rats with calvarial defects similar to the experiment described above. Hydrogels with and without RGD were tested, with hydrogels without exosomes used as controls. Results were assayed at 4 weeks and at 8 weeks post implantation by μCT. Results are shown in FIG. 76. The most significant regeneration results were seen in the alginate-RGD samples containing the BMP2 exosome indicating that engineered exosomes can be delivered using hydrogels that incorporate ECM derivative peptides as exosome carriers.

### Example 10: Engineering Functionally Enhanced MSC EVs for Regenerative Medicine

Mesenchymal stem cells (MSCs) are multipotent cells with regenerative and immunomodulatory properties. Several aspects of MSC function have been attributed to the paracrine effects of MSC derived extracellular vesicles (EVs). Recent studies suggest that the composition of MSC EVs is altered by the differentiation state of MSCs. However, the ability to control MSC EV functionality for tissue-specific regeneration has been elusive. The primary goal of this study is to evaluate the applicability of functionally enhanced MSC EVs for regenerative medicine. To achieve this, bone regeneration has been utilized as a proof-of-concept approach. This study elucidates that altering the MSC state by inducing differentiation into multiple lineages does not affect the endocytic property of the resulting EVs, but upon endocytosis, the EVs trigger the expression of lineage-specific genes and proteins in naïve MSCs in vitro and in vivo. Therefore, lineage-specific MSC EVs induced cell-type specific changes in target MSCs. To exploit this property for the generation of MSC EVs with consistent properties, genetically modified MSCs were generated by constitutively expressing BMP2 to generate EVs with osteoinductive properties. These EVs maintained the size distribution and endocytic characteristics of MSC EVs and showed enhanced bone regenerative potential compared to controls. Mechanistic studies revealed that the functionally enhanced EVs potentiate the BMP2 signaling cascade by delivering miRNA that suppress the negative regulators of BMP2 signaling. The results presented here collectively indicate that EVs may be engineered by genetic modification of the parental MSCs to induce lineage-specific differentiation and tissue regeneration in vivo. These effects seem to be primarily mediated via targeted pathway-specific changes to their miRNA cargo.

Mesenchymal stem cells (MSCs) are multipotent somatic stem cells that can be isolated from a variety of tissues such as the bone marrow, adipose tissue and dental pulp. The regenerative, protective and anti-inflammatory properties of MSCs especially bone marrow derived MSCs are well documented and make MSCs attractive cells for regenerative therapies. As of 2016, about 493 clinical trials that used MSCs were reported in the NIH clinical trials database. However, issues such as donor dependent variability, cellular viability, poor attachment and aberrant differentiation have posed significant hurdles for the use of MSCs in clinical treatment.

Many existing tissue-engineering approaches focus on delivery of selected proteins (growth factors, transcription factors etc.) or nucleic acids to host or implanted stem cells to achieve lineage specific differentiation. A variety of techniques ranging from exogenous addition of growth factors and controlled release devices to utilization of engineered biological and synthetic nano vesicles such as liposomes and polymeric vesicle have been investigated to deliver morphogens. Although the single morphogen system shows initial promise, when applied clinically, issues such as dosage, specificity, ectopic effects, toxicity, and immunological complications have posed significant restrictions to clinical efficiency as well as translational potential. Therefore, a sophisticated system that is biomimetic in nature provides necessary cues in physiologically relevant amounts and avoids the limitations of the single morphogen system is required. EVs/exosomes can satisfy these criteria.

EVs are nano vesicles (40-150 nm) secreted by cells to facilitate intracellular communication. As these vesicles pinch off the plasma membrane of cells, their lipid bilayer membrane is representative of the parental cell's plasma membrane. Within the EV, RNA (both mRNA and miRNA), cytosolic proteins as well as transmembrane proteins are present. These nano packets of information are endocytosed by effector cells to trigger a cellular response designated by the parental cell to the target cell. Although originally believed to be mediators of cellular homeostasis by secreting cellular waste, the past decade study of EVs demonstrate their specific roles in modulating cellular function in immunology, cancer biology and regenerative medicine.

Recent evidence suggests that several of the beneficial effects of MSC therapy can be attributed to paracrine effects of the MSC secretome. More specifically, MSC derived EVs have been implicated as the principal active agents of the MSC secretome. A recent study highlighted that MSC-derived EVs possess better anti-inflammatory properties compared to MSC derived microparticles. Recent studies have shown that bone marrow and dental pulp MSC derived EVs can be used to induce osteogenic and odontogenic differentiation of naïve MSCs respectively. Additionally, a recent study indicates that MSC EV function supersedes the extracellular matrix (ECM) derived signals indicating the potent nature of EV signaling. These and many other studies implicate MSC derived EVs as effective tools in clinical efforts to control inflammation and regenerative therapy and in the treatment of disease.

The paracrine aspect of MSC function involves the directed uptake of MSC derived EVs by target cells. Further, the multilineage differentiation potential of MSCs suggests that lineage specific function could be reflected as lineage specific exosomal effects on naïve target cells. Harnessing the fundamental mechanistic features of EV-mediated signaling can be turned into an application-specific tool to direct lineage specific tissue repair/regeneration and disease treatment. With these goals in mind, this study characterized basic mechanistic aspects of MSC EV function and applies it to generate engineered lineage-specific MSC EVs that are able to modulate tissue repair and regeneration using bone as a model system.

### Materials and Methods

Cell Culture:Human bone marrow derived primary MSCs (HMSCs) were purchased from ATCC and Lonza. These cells were cultured in aMEM (Gibco) containing 20% fetal bovine serum (FBS, Gibco), 1% L-Glutamine (Gibco) and 1% antibiotic-antimycotic solution (Gibco). For induction of differentiation of HMSCs into osteogenic, chondrogenic and adipogenic lineages, the growth medium was supplemented with growth factors and differentiating agents. Osteogenic differentiation was induced by culturing the cells in αMEM growth medium containing 100 μg/ml ascorbic acid (Sigma), 10 mM β-glycerophosphate (Sigma), and 10 mM dexamethasone (Sigma) for 4 weeks. Chondrogenic differentiation was induced by culturing the cells in αMEM basal medium containing 1 μM dexamethasone, 50 μg/ml ascorbate-2-phosphate (Sigma), 1% ITS premix (BD Biosciences), 1% F6S and 10 ng/ml TGFβ1 growth factor (Sigma) for 4 weeks. Adipogenic differentiation was induced by culturing the cells in growth medium containing 10 μg/ml insulin (Sigma), 500 μM isobutyl-I-methylxanthine (Sigma), 100 μM indomethacin (Sigma) and 1 μM dexamethasone for 4 weeks.

EV isolation and characterization: EVs were isolated from the culture medium as per standardized protocols. HMSCs were washed in serum free medium and cultured under serum free condition for 24 hours. If they were subjected to supplementation for altering cell state, the supplementation was maintained with only FBS being removed. The culture medium was harvested, cleaned of cell debris by centrifugation (1000×g) and EVs were isolated using the ExoQuick TC isolation reagent (System Biosciences) as per the manufacturer's recommended protocols. To maintain consistency, the isolated EVs were resuspended in PBS such that each 100 μl of EV suspension contained EVs from approximately 1×10⁶ HMSCs. This equated to a stock concentration of 10,000 particles/μl as determined by nanoparticle tracking analysis (NTA).

The isolated EVs were characterized for number and size distribution and presence of membrane markers by NTA, immunoblotting and transmission electron microscopy (TEM) as per established standards. For NTA, a 1/100 dilution of the EV suspension was analyzed in the Nanosight NS-300 instrument to obtain the size distribution plot. For quantitative experiments, the EV concentration (particles/nil) was also measured by NTA and equal number of EVs were used for each experiment.

For immunoblotting, exosomal proteins were isolated in RIPA buffer and 10-20 μg of EV protein isolate was resolved by SDS-PAGE, transferred onto nitrocellulose membranes and probed with primary rabbit anti-CD63 (1/500, Abcam) and mouse anti-CD9 (1/500, Abcam), mouse anti BMP2 (1/500, Abcam) antibodies and near infrared dye conjugated secondary antibodies (1/10,000 Licor). The blots were then dried and imaged using a Licor Odyssey imager. For immunoblotting of the conditioned medium, the medium from which EVs were isolated was dialyzed against deionized water, lyophilized and reconstituted in 1× lameli buffer. SDS PAGE and immunoblotting were performed.

For transmission electron microscopy (TEM), 10 μl of 1/10 dilutions of the EV suspensions were placed on to carbon fomvar coated nickel TEM grids and incubated for 1 hour followed by fixing with 10% formalin, washing with double deionized water and air drying. For immunogold labeling of CD63, the EV containing grids were blocked in PBS with 5% BSA, incubated with CD63 antibody (1/100, Abcam) followed by washing and incubation with 10 nm gold tagged secondary antibody (1/1000, Abcam). The grids were then washed and air-dried. All the grids were imaged using a Joel JEM3010 TEM.

### Quantitative and Qualitative Endocytosis of MSC EVs:

For endocytosis experiments, MSC EVs were fluorescently labeled using the ExoGlow green labeling kit (System Biosciences) that labels the exosomal proteins fluorescently. The EVs were resuspended in PBS with 100 μl corresponding to EVs from 1 million MSCs.

For quantitative experiments HMSC cells were plated on to 96 well tissue culture plates at a concentration of 10,000 cells per well and incubated for 18 hours to facilitate cell attachment. The cells were then incubated with increasing amounts of fluorescently labeled HMSC EVs for 2 hours at 37° C. The cells were washed with PBS and fixed in neutral buffered 4% paraformaldehyde. The fluorescence from the endocytosed EVs was measured using a BioTek Synergy2 96 well plate reader equipped with the appropriate filter sets to measure green fluorescence. The results were plotted as mean (+/−SD) normalized fluorescence intensities (normalized to background and no EV fluorescence) as a function of dosage (n=6 per group).

For quantitative endocytosis blocking experiments, the cells were plated in 96 well plates or in 12 well culture plates (50,000 cells/well) and prior to EV treatment, were pre-treated with the blocking agents for 1 hour. Cell surface integrins were blocked with 2 mM RGD polypeptide (Sigma). Membrane cholesterol was depleted using methyl β cyclodextrin (MBCD, Sigma) in a dose dependent manner (0-10 mM). In addition to this treatment, the labeled EVs were pretreated for 1 hour with indicated concentrations of heparin (0-10 μg/ml, Sigma) to block the heparin sulfate proteoglycan binding sites on the exosomal membrane. For the qualitative and quantitative experiments, the fluorescently labelled exosomal volume was maintained at 2× saturation volume (determined from the saturation curve. The stock concentration of EV was 10,000 particles/μl) to ensure that saturable levels of HMSC EVs are used in the assay. Treatment with the EV suspension was carried out and the fluorescence measurement and quantitation and statistical analysis was performed as per published protocols.

For qualitative endocytosis experiments, 50,000 cells (HMSCs) were plated on coverslips placed in 12 well tissue culture dishes. Fluorescently labeled EVs at 2× saturation volume were then added with/without inhibitors as described above and incubated for 2 hours in the presence/absence of blocking agents as described above. The cells were then washed, fixed in 4% neutral buffered paraformaldehyde, permeablized and counter stained using mouse monoclonal anti tubulin antibody (1/2000, Sigma), rabbit polyclonal anti caveolin1 antibody (1/100, Santacruz Biotechnology) or rabbit polyclonal anti clathrin antibody (1/100, Santacruz Biotechnology) followed by treatment with TRITC labeled anti mouse/rabbit secondary antibody. The coverslips were then mounted using mounting medium containing DAPI (Vector Labs) to label the nuclei and imaged using a Zeiss LSM 710 Meta confocal microscope.

EV mediated HMSC differentiation: HMSCs were differentiated as described under the cell culture methods section and EVs from the differentiated HMSCs were isolated as described under the isolation section. The isolated EVs were characterized for size and the presence of exosomal markers as described under the characterization section. For in vitro differentiation experiments, naïve HMSCs (250,000 cells per 1 cm×1 cm hydrogel) were embedded in type I collagen hydrogels in quadruplicates. Clinical grade collagen sponges (Zimmer collagen tape) were used as the hydrogel of choice. 2× saturation volume of the different EVs (osteogenic, chondrogenic and adipogenic) were then added to the cells and incubated for 72 hours. The saturation volume was determined by the quantitative dose dependence endocytosis experiment described in the previous section. The saturation was reached at 20 μl of standardized EV suspension per 10,000 HMSCs. NTA was used to measure the amount of EVs and this amounted an average of 10,000 EV particles/μl of standardized EV suspension from HMSCs. 1×10⁸ EV particles were used per group in this experiment. Untreated cells received PBS treatment of equal volume. Post 72 hours, RNA was isolated from the embedded HMSCs followed by cDNA synthesis and qPCR for selected marker genes for osteogenic, chondrogenic and adipogenic differentiation as published protocols and primer sequences.

Generation of BMP2 Overexpressing HMSCs and their EVs:

Lentiviral particles containing a mammalian dual promoter vector that encodes the BMP2 gene under the control of EF1α promoter and a GFP marker under the control of SV40 promoter or control vector without the BMP2 gene was obtained from Applied Biological Materials (ABM). HMSCs were transfected with the lentiviral particles as per the manufacturer's instructions and stably selected using puromycin. EVs were isolated and characterized from these overexpressing and control cells and the ability of these EVs to induce HMSC differentiation was evaluated.

SMAD 1/5 specific reporter assay: 30,000 HMSCs cultured in 24 well tissue culture plates were transfected in quadruplicates with control or SMAD 1/5 specific luciferase reporter plasmid (SBE12(31)) using lipofectamine transfection reagent. 48 hours post transfection, the cells were treated with the control or experimental reagents in quadruplicates. The EVs were added at 2× saturation dosage. This amounted to 6×10⁶ EVs for every 30,000 HMSCs. 48 hours post transfection, total protein was extracted from the cells, concentration determined and the luciferase activity from equal amounts of protein for each sample from each group was measured (reporter kit Promega) and normalized to control activity. The data is represented as mean % increase in luciferase activity (+/−SD, n=4) w.r.t untreated cells expressing the SMAD1/5 reporter and statistical significance was calculated using student's t-test.

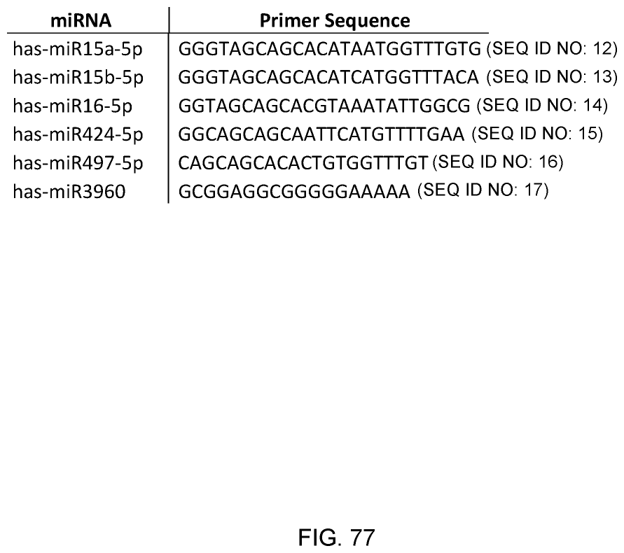

Quantitative miRNA expression in EVs: qRT PCR was used to evaluate the expression level of miRNAs in the exosomes. The miRNA was isolated from equal numbers of control and BMP2 EVs using the Qiagen miRNA isolation kit as per the manufacturer's protocol. cDNA synthesis was performed using the miScript II kit (Qiagen) and qRT PCR was performed using the SYBR greet PCR kit (Qiagen) using custom primers for the selected miRNA (FIG. 77). As there is no defined housekeeping miRNA for EVs, direct quantitation was performed by utilizing exact amounts of small RNA from equal numbers of EVs for all groups for cDNA synthesis followed by quantitation of the cDNA amounts and double standardization to obtain the fold change in expression levels. The data is represented as mean fold change (n=4). Statistical significance was calculated between the control and BMP2 EV samples using student's t-test.

Mouse subcutaneous implantation experiments: All in vivo experimentation was performed in either immunocompromised mice (Charles River Labs, 1-month old mice) or Sprague Dawley rats (250-300 g, Charles River Labs) as per protocols and procedures approved by the University of Illinois animal care committee (ACC). All animals were housed in appropriate cages in temperature and humidity-controlled facilities. Food and water were made available at libitum.

The ability of EVs from differentiated HMSCs to induce lineage specific differentiation of naïve HMSCs was evaluated in vivo in an immunocompromised mouse subcutaneous implantation model. 1×10⁶ HMSCs were seeded on to a 1 cm×1 cm square of clinical grade collagen tape (Zimmer) with 2× saturation volume (approximately 4×10⁸ EVs) of respective control (naïve HMSC EV) or experimental EV (osteogenic, chondrogenic or adipogenic) suspension and implanted within the subcutaneous pocket bilaterally on the back of immunocompromised mice. The mice were anesthetized by intraperitoneal injection of Ketamine (80-100 mg/kg)/Xylazine (10 mg/kg). A 1.5 cm incision was made on the back along the midline and the control or experimental scaffolds were placed bilaterally within the subcutaneous pocket. All experiments were performed in quadruplicate. 4 weeks post implantation, the animals were sacrificed by carbon dioxide asphyxiation followed by cervical dislocation. The scaffolds were extracted, fixed in neutral buffered 4% paraformaldehyde, embedded in paraffin and sectioned in to 5 μm sections. The sections were then immunostained fluorescently for marker proteins, mounted and imaged using a Zeiss LSM 710 laser scanning confocal microscope. All primary antibodies were purchased from Abcam and were used at a dilution of 1/100 of the stock solution. The secondary anti-mouse FITC and anti-Rabbit TRITC were obtained from Sigma and were used at a dilution of 1/200.

Rat calvarial bone defect model: To evaluate the ability of HMSC derived EVs to regenerate bone, a rat calvarial defect model was used. All groups and time points contained 6 repeats. Briefly, the rats were anesthetized intraperitoneally using Ketamine (80-100 mg/kg)/Xylazine (10 mg/kg). Using aseptic technique, a vertical incision was made in the head at the midline to expose the calvarial bone. The connective tissue was removed and two 5 mm calvarial defects were created bilaterally in the calvarium without dura perforation using a trephine burr. A clinical grade collagen tape (Zimmer) was placed on the wound with or without control or experimental EVs. The amount of EVs used was 5×10⁸ EVs per defect. Collagen tape alone served as control and rhBMP2 (50 μg/wound, Medtronic) containing scaffolds served as positive control. Four, 8- and 12-weeks post-surgery, the rats were sacrificed by carbon dioxide asphyxiation followed by cervical dislocation. The calvaria were harvested, fixed in neutral buffered 4% paraformaldehyde and subjected to 3D μCT analysis using a Scanco40 μCT scanner. The data obtained from the μCT scanner was quantitatively analyzed using a custom built Matlab Program. The samples were then decalcified in 10% EDTA solution, embedded in paraffin, sectioned into 10 μm sections and subjected to histology.

### Results

### Characterization of EVs:

EVs isolated from HMSCs were characterized for size, shape and presence of exosomal marker proteins. NTA analysis indicated that the isolated vesicles show a particle size distribution consistent for EVs (FIG. 78A). On average, after the standardized EV dilution (100 μl suspension containing EVs from 1×10⁶ cells), the EV concentration for HMSCs used was determined to be approximately 1×10⁸ particles/ml of the EV suspension. Electron microscopy analysis revealed spherical vesicles between 100-150 nm in size. Osteogenic, chondrogenic and adipogenic differentiation of HMSCs yielded EVs that shared similar vesicle size distribution (FIG. 78A). The TEM morphology and size also remained consistent between undifferentiated and differentiated HMSC derived EVs (FIG. 78B). Immunoblot analysis indicated the presence of exosomal marker proteins CD63 (FIG. 78C) and CD9 (FIG. 78D) in both naïve and differentiated HMSC EVs. Taken together, these results indicate that the extracellular vesicles isolated from HMSCs here, conform to accepted properties of EVs and that these physical characteristics remain unchanged irrespective of the differentiation state of the source HMSCs.

### Endocytosis of HMSC Derived EVs:

### a) Different Cell Types Show Similar Endocytosis of HMSC EVs:

EVs from different cell types have been shown to be endocytosed by a variety of mechanisms. The endocytic mechanism of HMSC EVs by target HMSCs was evaluated. HMSC EV endocytosis by MSCs was a dose dependent and saturable process (FIG. 79A). Pretreatment of the EVs with heparin significantly reduced the endocytosis (FIG. 79B, 79F) suggesting the involvement of membrane surface heparin sulfate proteoglycan receptors (HSPGs) in the process of EV endocytosis. Pre-treatment of the target cells with 2 mM RGD peptide to block the cell surface integrins did not completely block EV endocytosis (FIG. 79G), indicating that integrins are not primary receptors involved in HMSC EV endocytosis. When endocytosis experiments were performed after pre-treatment with MBCD to disrupt the membrane cholesterol, EV endocytosis was significantly reduced, indicating the involvement of the lipid raft/caveolar pathway (FIG. 79C). Further, colocalization experiments with caveolin1 (a marker protein for caveolae) and clathrin (marker protein for clathrin coated pits) indicated that the fluorescently labeled EVs co-localized with caveolin1 (FIG. 79H) and not clathrin (FIG. 79I). Finally, when endocytosis experiments were performed at 4° C., EV endocytosis was blocked indicating the temperature and thereby, the energy dependency of the process (FIG. 79E). Overall, these results indicate that MSC EV endocytosis was a dose dependent and energy dependent process and occurs in a heparin-sensitive manner that is mediated via the caveolar endocytic pathway.

b) EVs from Differentiated HMSCs are Endocytosed by Naïve HMSCs:

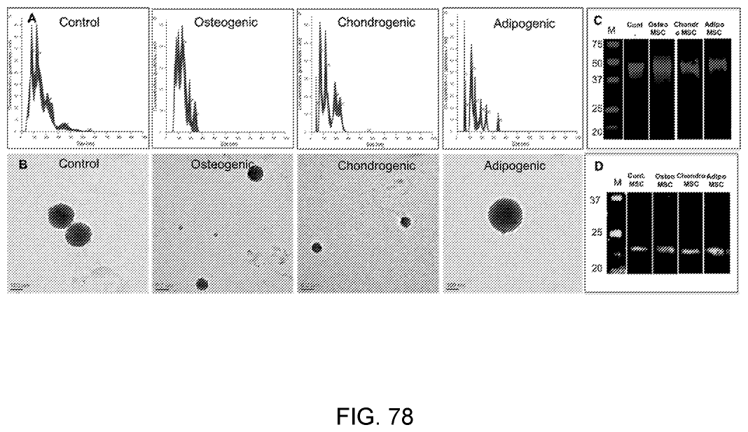

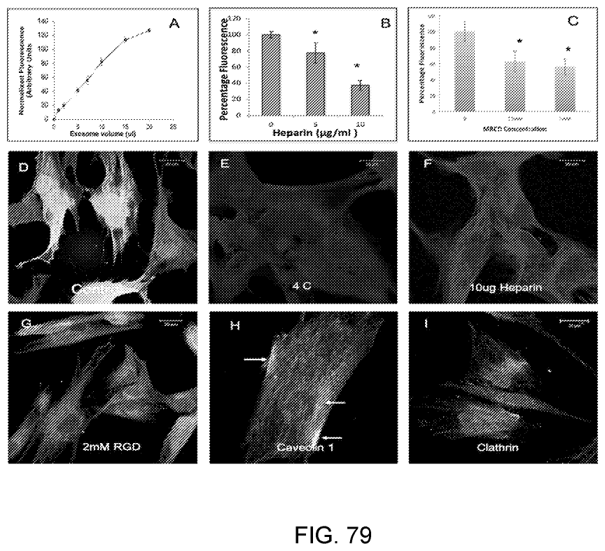

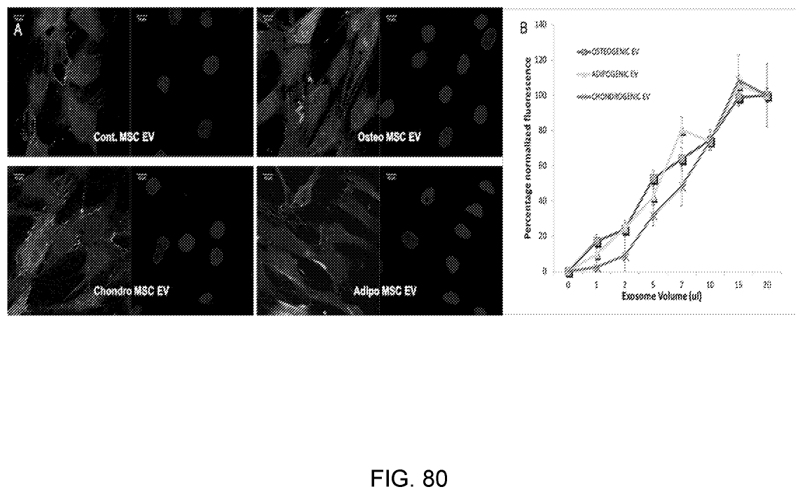

Because a common mode of endocytosis occurs across multiple cell types, a change in cell state was tested to determine if this variable would affect the endocytosis of lineage-specified, HMSC derived EVs. HMSCs were first differentiated along the osteogenic, chondrogenic and adipogenic lineages. EVs isolated from these cells were harvested and evaluated for dose dependent and saturable endocytosis. FIG. 80A shows representative confocal images of the different fluorescently labeled EVs by naïve HMSCs. Further, the dose-dependent endocytosis of the multi-lineage EVs by naïve HMSCs was similar without any statistically significant difference irrespective of the HMSC lineage from which EVs were isolated (FIG. 80B).

EVs from Differentiated HMSCs Induce Lineage Specific Differentiation of Naïve HMSCs in Vitro and In Vivo:

Undifferentiated HMSCs in 3d cultures were incubated with EVs isolated from differentiated HMSCs for 72 hours. Osteogenic, chondrogenic and adipogenic EVs induced a significant increase in the expression levels of respective lineage specific marker genes with respect to untreated controls (FIG. 81). These genes included a mixture of growth factors, transcription factors and ECM proteins representative of the individual lineages. The genes represented in FIG. 81 for each of the three lineages were unique to that specific lineage such that they were not significantly affected by other MSC EVs. For example, the osteogenic genes represented here did not show a statistically significant change when naïve HMSCs were treated with chondrogenic or adipogenic MSC EVs. This result indicates the specificity of action.

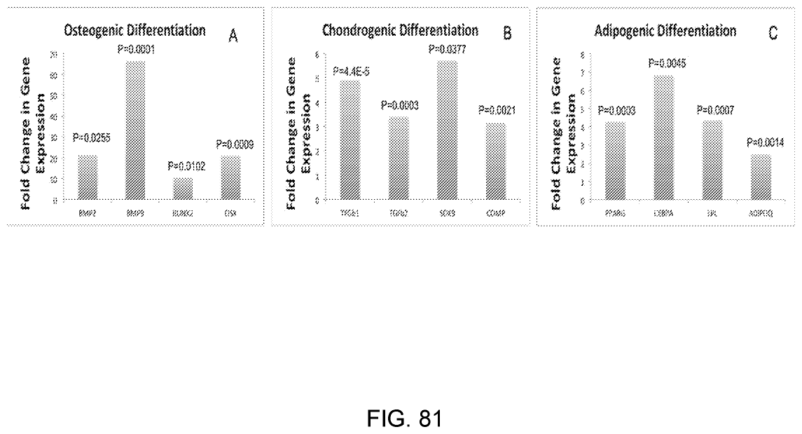

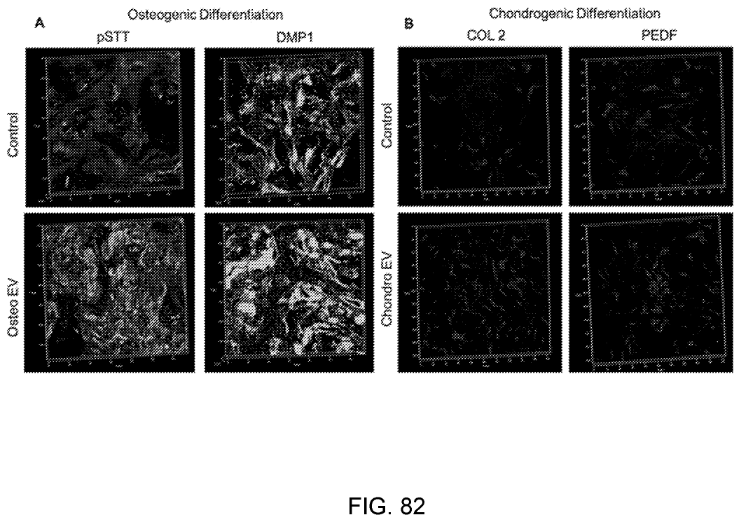

To verify these effects in vivo, collagen sponges loaded with undifferentiated HMSCs with or without EVs were implanted subcutaneously in the back of immunocompromised mice. After 4 weeks, the forming tissue were excised, fixed, embedded and the sections were analyzed by fluorescence immunohistochemistry for the expression of lineage-specific marker proteins. For all three different EVs, lineage-specific protein expression was observed. FIG. 82 shows representative confocal images of the sections.

For osteogenic differentiation the expression levels of phosphorylated proteins and dentin matrix protein 1 (DMP1) were analyzed. Phosphorylated proteins were analyzed by staining the sections with an antibody that recognizes phosphorylated serine, threonine and tyrosine residues. Phosphorylated proteins serve as a source for organic phosphorus in osteogenic environments aiding the nucleation of calcium phosphate by serving as substrates for phosphatases. DMP1 is an osteogenic marker protein that is involved in osteoblast differentiation and hydroxyapatite nucleation. Results presented in FIG. 82 show that HMSCs from the group treated with osteogenic EVs showed an increased presence of phosphorylated proteins and increased expression of DMP1 compared to the control adding evidence to the in vitro results presented in FIG. 81.

Similarly, chondrogenic differentiation was evaluated by looking at the expression levels of type II collagen, a major component of the cartilaginous matrix as well as the expression level of pigment epithelium derived factor (PEDF). PEDF is a potent anti-angiogenic factor that is expressed in developing cartilage tissue to actively prevent vascularization. Results presented in FIG. 82 show that type II collagen and PEDF expression was elevated in HMSCs subjected to chondrogenic EV treatment with respect to control HMSCs.

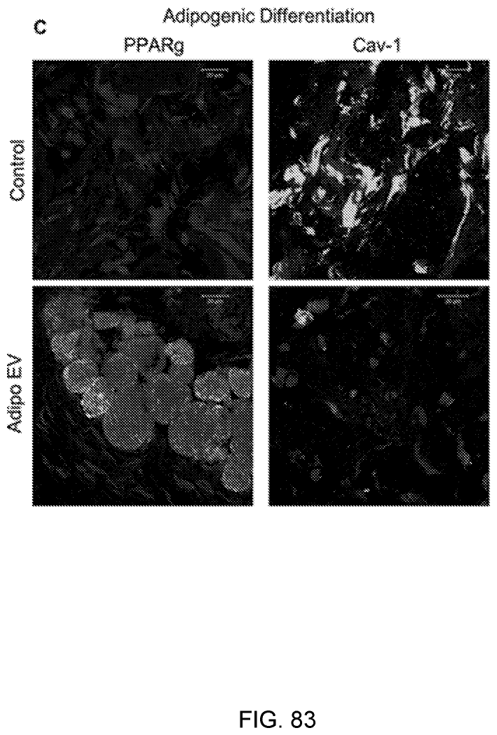

Finally, adipogenic differentiation of HMSCs from the subcutaneous implants was evaluated by evaluating the expression levels of peroxisome proliferator activator receptor-gamma (PPAR-γ) and caveolin 1. PPAR-γ is a nuclear receptor that controls adipogenesis and adipogenic differentiation of MSCs. On the other hand, caveolin 1 expression is reduced upon induction of adipogenic differentiation of MSCs. Results presented in FIG. 83 show an increased expression of PPAR-γ and reduced expression of caveolin 1 in HMSCs treated with adipogenic EVs compared to controls indicating an induction of adipogenic differentiation. Additionally, these cells demonstrate the presence of fat-like deposits with positive PPAR-γ staining.

Collectively, these results indicate that EVs isolated from differentiating HMSCs can induce lineage-specific phenotypic changes in naïve HMSCs in vitro and in vivo. EVs from BMP2 overexpressing HMSCs can enhance differentiation in vitro and bone regeneration in vivo:

Based onobservations that lineage-specificity is imparted to HMSC EVs with a functional impact upon target cells, it was speculated that genetic manipulation of HMSCs serving as an EV source could generate EVs with enhanced functionality for targeted differentiation of stem cells. To explore this possibility and to investigate the potential of generating standardized EVs from a stabilized parental cell line, a stable HMSC line that constitutively overexpresses BMP2 (BMP2 OE HMSCs) was generated. This cell line demonstrated increased mRNA expression of BMP2 compared to control (untreated) and vector control cell lines (FIG. 84A). The BMP2 expression was further associated with functional differentiation; FIG. 84B shows a representative image of the control, vector control and BMP2 OE HMSCs subjected to cell culture in 6 well dishes in the presence of osteogenic differentiation media (7 days) and stained for alizarin red to identify calcium deposits. The BMP2 OE HMSCs generated higher amounts of calcium deposits compared to the controls indicating their greater osteogenic differentiation potential.

EVs were isolated from these BMP2 OE HMSCs (BMP2 EV) and evaluated for the presence of marker protein CD63 by immunoelectron microscopy (FIG. 84C), size distribution by NTA (FIG. 84D) and for endocytosis by naïve HMSCs quantitatively (FIG. 84E). Results presented in FIGS. 84C, 84D and 84E indicate that the isolated EVs possess a similar size distribution as the control and differentiated HMSC EVs (compare to FIG. 79) and are endocytosed by naïve HMSCs in a dose dependent manner similar to the control HMSC derived EVs (compare to FIG. 79A).

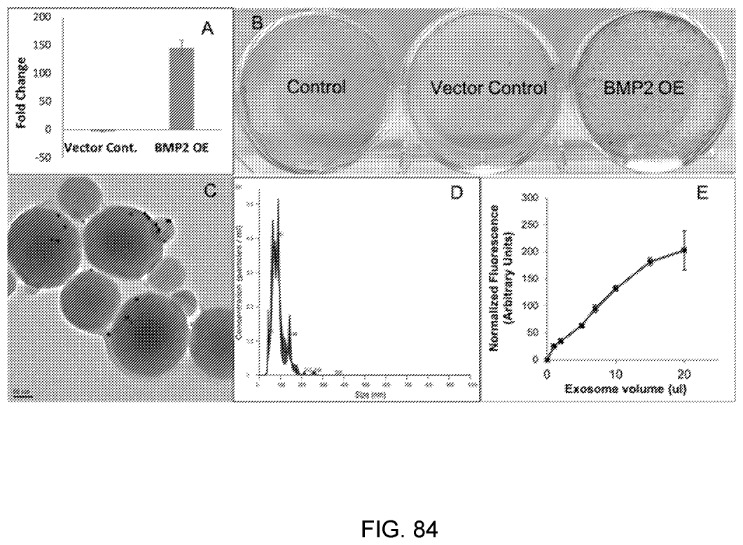

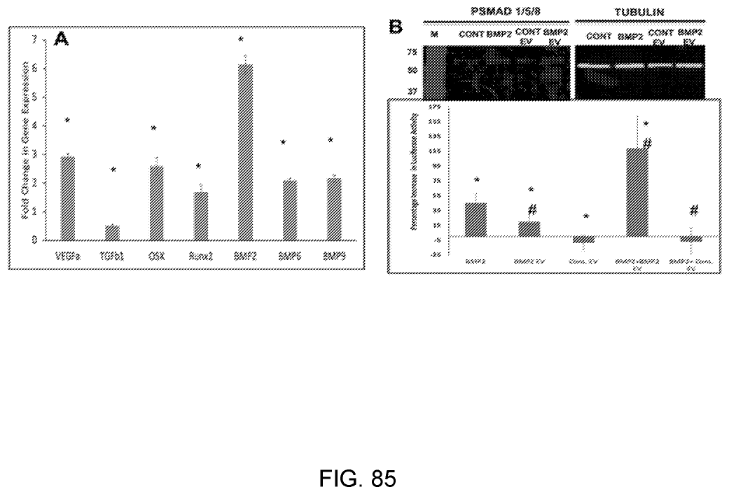

To explore whether the induced lineage-specification of HMSCs altered the function of these EVs, their potential to induce osteogenic differentiation of naïve HMSCs in vitro was evaluated. Results presented in FIG. 85A show that BMP2 EV treated HMSCs showed a significant increase in the expression of osteogenic marker genes. As the EVs were isolated from BMP2 overexpressing cells, the study sought to evaluate if the EVs themselves trigger the BMP2 signaling cascade. To test this, HMSCs were subjected to a 4 hr incubation with control EVs, BMP2 EVs and with rhBMP2 (positive control) and evaluated for phosphorylation of SMAD1/5/8. Untreated HMSCs remained as baseline. Results presented in FIG. 85B indicate that treatment with either rhBMP2 and BMP2EVs triggered SMAD 1/5/8 phosphorylation and treatment with control HMSC EVs did not indicating that the BMP2 EVs were triggering the BMP2 signaling cascade. To further confirm this effect, HMSCs were transfected with a reporter luciferase construct that is specific to SMAD 1/5 activity and evaluated for response. Results presented in FIG. 85B indicate that the luciferase activity was increased upon treatment with positive control BMP2 and to a lesser extent with BMP2 EV. Interestingly, when the EVs were used in combination with rhBMP2, a robust increase in luciferase activity was observed with the BMP2 EVs but not with control EVs indicating that the BMP2 EVs were potentiating the BMP2 signaling cascade. Additionally, the presence of control EVs actively negated the effect of rhBMP2.

To provide assurance that the BMP2 EV effects was not the result of BMP2 protein expression from the parental cell, both the EVs and EV depleted conditioned media were examined for BMP2 and EV marker CD63 expression. FIG. 86C shows the result from this experiment. BMP2 was not present in detectable levels in the conditioned medium from control cells and in the EV protein extracts from both control and BMP2 groups. However, BMP2 was detected in the EV depleted conditioned medium from the BMP2 OE HMSCs (visible band in lane 2 of FIG. 86C). On the other hand, CD63 (labelled) was present only in the EV protein extracts from both groups. Overall, this result indicated that BMP2 protein was not packaged with in the EVs of the BMP2 OE HMSCs.

The next experiment sought an exosomal miRNA-based mechanism that enables BMP2 EVs to potentiate the BMP2 signaling pathway. To identify possible miRNA targets, TargetScan (targetscan.org) was used to identify miRNA targets that might bind to the negative regulators of the BMP2 pathway namely SMURF1 and SMAD7. Interestingly, a cluster of five miRNAs that bind to the 3′ untranslated region (UTR) of both SMURF1 and SMAD7 was identified. These miRNAs are broadly conserved among vertebrates, indicating their importance in the control of the BMP2 pathway. To demonstrate that these miRNAs were differentially expressed among control and BMP2 EVs, the miRNA levels in control and BMP2 EVs were analyzed by qRT PCR. Results presented in FIG. 86D indicate a statistically significant increase in the levels of these miRNA in the BMP2 EVs compared to control HMSC EVs. On the other hand, there was no significant change in the expression level of miR 3960, an miRNA that has been implicated in osteogenic differentiation and bone regeneration via regulation of RUNX2 gene. Taken together, these results indicate a pathway-specific mechanism active in these lineage-specific, functionally enhanced exosomes.

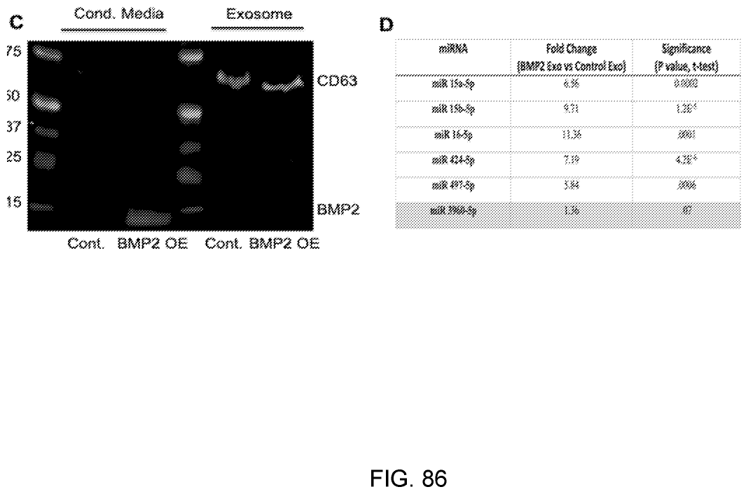

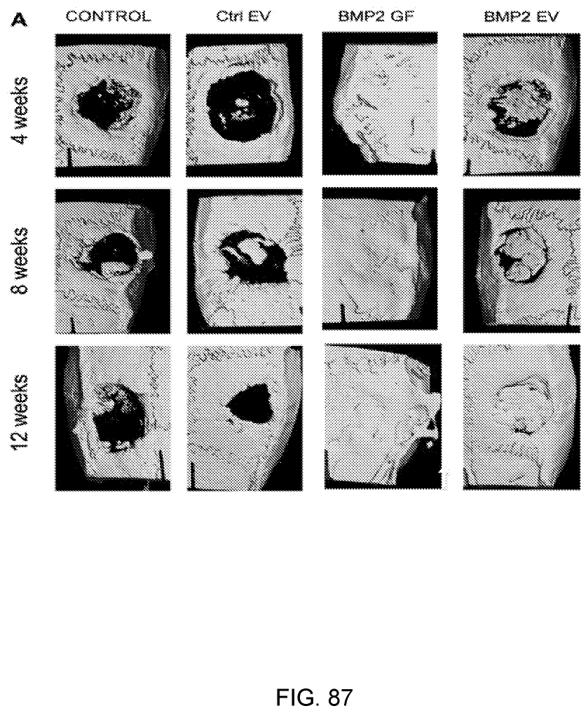

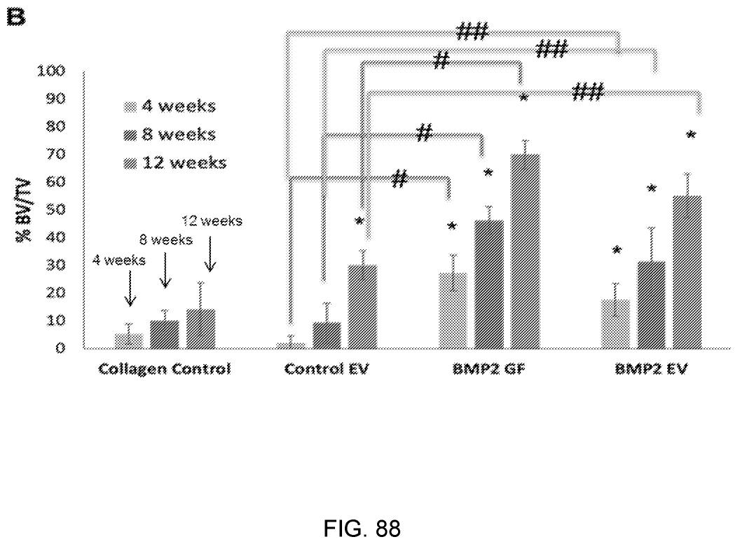

Finally, the functionality and translational relevance of BMP2 EVs was evaluated in vivo in a rat calvarial defect model. FIG. 87 shows representative 3D reconstructed μCT images of rat calvaria after 4, 8- and 12-weeks post wounding. For these experiments, rhBMP2 was used as a positive control. rhBMP2 induced a rapid and robust bone growth over 12 weeks compared to the other groups. At this high, effective dose, bone formation obliterated the calvarial sutures and areas of ectopic bone formed (12-week group white arrow). In contrast, the group of rats treated with EVs from BMP2 OE cells (BMP2 EV) showed a gradual increase over time in bone formation followed by robust wound coverage by 12 weeks. Mineralized bone formation appeared to be exclusively confined to the treated defect region. The control groups (No EV and naïve HMSC EV (Control EV)) showed minimal healing over the study period. The μCt data was quantified using a custom designed Matlab program that evaluates BV/TV ratios as percentage of defect volume filled with mineralized tissue at the different time points. The results of this quantification are presented in FIG. 88. These results show that the healing of cranial defects in the BMP2 EV group was significantly greater than either control group indicating that the application of the BMP2 EVs enhanced osseous regenerative function. Thus, this demonstrates the potential for EVs from an engineered lineage-specific cell line to provide instruction for lineage-specific regeneration.

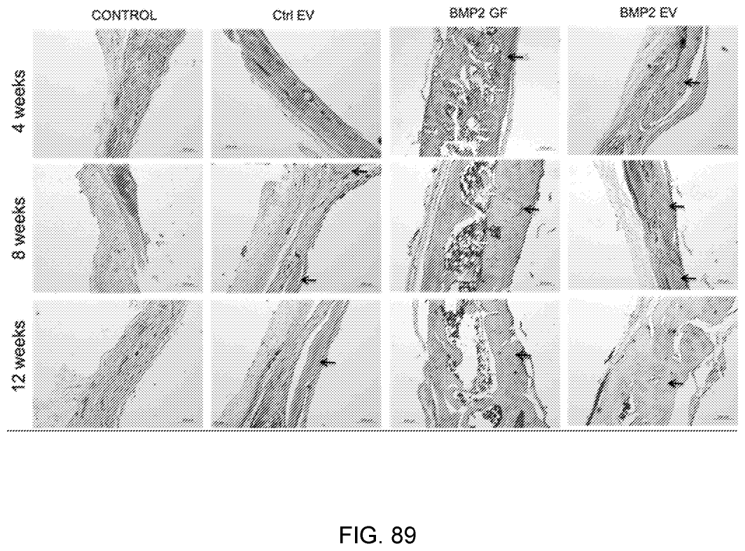

Histological evaluation was performed on paraffin embedded sections of demineralized tissues across all groups and time points. Results presented in FIG. 89 validate the incomplete and poor healing observed in the control groups over the different time points evidenced by the increased presence of connective tissue and minimal bone matrix. In contrast, both the BMP2 EV and the rhBMP2 groups showed greater regeneration of bone tissue. The histological sections corroborate the μCT data indicating the comprehensive regeneration of bone tissue in the rhBMP2 group. Notably, the BMP2 EV group histology revealed ongoing woven bone formation across the defects, indicating a dedicated intramembranous bone regeneration process was induced.

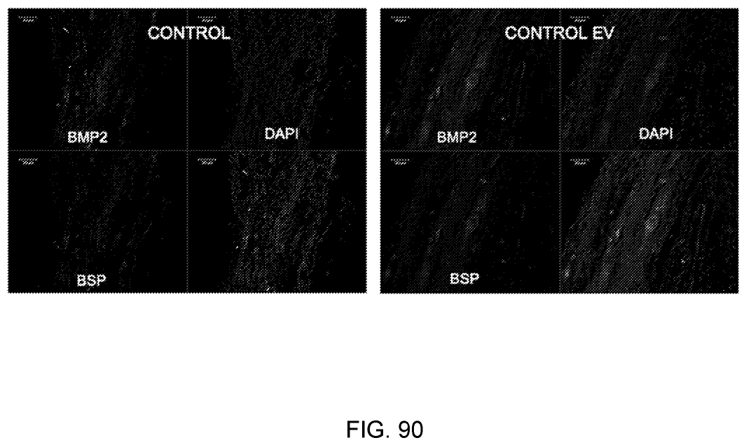

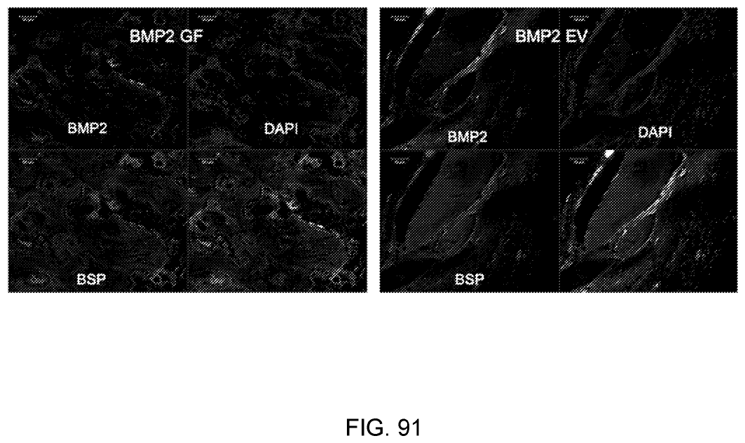

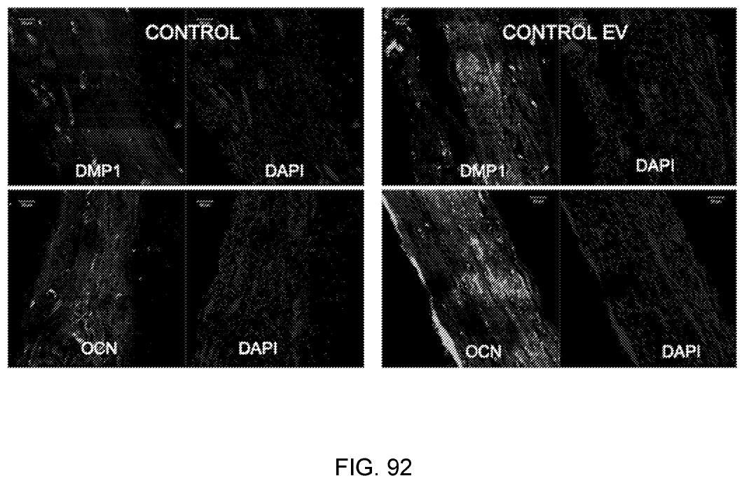

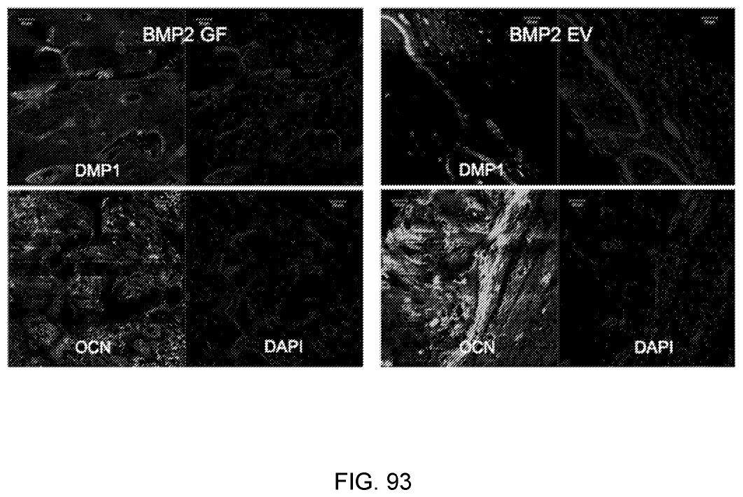

Further, immunofluorescence staining was performed on the 4 week sections from the different groups to evaluate the expression levels of proteins important for bone formation. Results presented in FIGS. 90-93 indicate that both rhBMP2 and the BMP2 EV groups induced early expression of BMP2, bone sialoprotein (BSP), dentin matrix protein 1 (DMP1) and osteocalcin (OCN). Taken together, the μCT and histological results indicate that EVs from a lineage-specified HMSC cell line (BMP2 OE HMSCs) are able to inform and target endogenous cells to differentiate along a parallel lineage to achieve tissue regeneration by a mechanism that enhances osteoinduction.

### DISCUSSION

Regenerative strategies require the recruiting and instructing of cells to form new tissues. MSC EVs are of current interest because they demonstrate immunomodulatory and regenerative potential that may rival the use of MSCs or growth factors in regenerative medicine. Furthermore, studies are currently underway to engineer MSCs to improve their ability to produce EVs by altering several secretory pathways. The immunomodulatory, angiogenic and regenerative potential of MSC EVs is well documented. The potential of bone marrow derived MSC EVs in bone regenerative applications has been demonstrated.

This study provided insights into some of the basic properties of MSC derived EVs and how they may be utilized and exploited for improving tissue engineering strategies. The inquiry began by investigating MSC EV endocytosis, a first requisite step in the process of EV-mediated paracrine signaling. Identification of the endocytic mechanism can provide valuable information to target EVs for therapeutic delivery. As the exosomal membrane is the subset of the plasma membrane of the source cell, EVs from different cell types undergo endocytosis via different mechanisms. The clathrin pathway, caveolar pathway, phagocytosis and even macropinocytosis have all been implicated in endocytosis of EVs. Energy dependence and dose dependence were observed, as well as dependence on membrane cholesterol, indicating the involvement of the lipid raft/caveolar endocytic pathway. Furthermore, the data shows that the MSC EVs are endocytosed in a manner that involves the target cell surface HSPGs. Based on observations made with dental pulp MSC derived EVs, this appears to be a common endocytic mechanism for MSC derived EVs. Further studies using different MSC sources are required to conclusively determine if this mechanism is applicable to MSCs in general.

Next, the study sought to explore an important question regarding the use of EVs for therapeutic purposes: Does the state of the parental cell influence a) exosomal properties and endocytic mechanism and b) exosomal cargo and function?” The results presented here show that the characteristics of MSC derived EVs are not altered by changes to cell state. When HMSCs were differentiated into osteogenic, chondrogenic and adipogenic lineages, the secreted EVs from these cells maintained their morphology, size distribution and expression of exosomal surface markers. From a therapeutic perspective, this result shows that modifications to MSC state may not adversely affect the properties of the secreted EVs.

Next, the study tested if lineage-specification of parental MSCs would inform the differentiation potential of EVs. Results indicated that the endocytic efficiency of MSC EVs is not altered by changes to cell state. EVs isolated from osteogenic, chondrogenic and adipogenic MSCs did not show any significant difference in their dose-dependent ability to be endocytosed by naïve MSCs. However, they were able to effect lineage specific changes within the target MSCs in vitro and in vivo. This effect can be due to the alterations to the exosomal cargo of miRNA, mRNA and proteins. The characterization of lineage specification by EVs from lineage differentiated MSCs underscores the unique character of cell-type specific EVs. This novel finding that directing tissue-specific regeneration using EVs from differentiated MSCs has wide-ranging applications in regenerative medicine.

Importantly, this work sought to create a stable cellular source for generating function-specific EVs for tissue engineering applications. Using bone regeneration as a model system, it was hypothesized that the stable transduction of HMSCs with an osteoinductive factor can generate a stable cell line to consistently produce lineage specifying EVs. To test this hypothesis, an HMSC cell line that overexpresses BMP2 was generated. BMP2 is a clinically used morphogen for bone regenerative procedures in orthopedic and dental surgeries that is not without identified complications or side effects. However, it is reproducibly efficient in the generation of bone in preclinical models including the model used here. EVs from the BMP2 OE HMSCs showed a similar size distribution, morphological and endocytic profile to that of naïve and differentiated MSC derived EVs indicating that genetic modification of the MSCs did not affect the basic properties of the secreted EVs. This is an important observation of this study that shows that genetic modification of source MSCs do not alter the properties of their derivative EVs. It is to be noted here that endocytic efficiency refers to the saturation amount of EVs and not the absolute value of fluorescence as this value is arbitrary and is subject to change with experimental conditions.

When analyzed for their osteoinductive potential in vitro, BMP2 EVs triggered osteogenic gene expression in naïve HMSCs. Pathway studies indicated that the BMP2 EVs potentiated the BMP2 signaling cascade. However, this activity was not due to BMP2 protein presence within the EVs. The results indicate that the increased osteoinductive potential of the BMP2 EVs is due to the increased levels of pathway specific miRNA within the EVs that negatively regulate the negative regulators of the BMP2 pathway in SMURF1 and SMAD7. Further refinement can enable changes to targeted pathways and enhance therapeutic specificity.

In a rat calvarial defect model, the BMP2 EVs performed significantly better than control groups that included calvarial wounds covered with just collagen sponge and collagen sponge containing EVs from control MSCs. Apart from highlighting the enhanced potential of the engineered MSC EVs, the data also revealed that EVs from undifferentiated MSCs possess limited bone regenerative potential. The bone formed in the BMP2 OE EV group is representative of intramembranous woven bone. The cell-rich mineralized matrix deposition at 4-12 weeks indicates that the EVs may be functioning by direct targeting of osteoprogenitors. Unlike rhBMP2 regenerated tissues, there is no ectopic and exaggerated bone formation nor an excessive vascular or adipogenic response to BMP2 EV stimulated bone regeneration. The involvement of various cells and the targeting of individual cell types by EV treatment in this model remains to be elucidated. In terms of the percentage volume of defect covered by mineralized tissue, the BMP2 Exo group performed admirably albeit not as robust as the rhBMP2 group. Collectively, the results from the bone regenerative experiments indicate that engineered EVs from genetically transformed MSCs can be used as mediators of host response to injury to improve regenerative outcomes.

Overall, the data presented in this study indicates that altering the MSC cell state generates EVs with function-specific properties without altering EV characteristics, size distribution or endocytic ability. EVs from genetically modified MSCs (BMP2) displayed unaltered size and endocytic properties compared to naïve MSC EVs but showed enhanced regenerative potential in vitro and in vivo in line with the targeted genetic modification. Furthermore, overexpression of BMP2 growth factor in MSCs altered the EV cargo to contain miRNA that potentiates the BMP2 signaling cascade. These results show how properties of MSC derived EVs may be manipulated for various applications in disease treatment and regenerative medicine.

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

1. A composition comprising isolated engineered exosomes from mesenchymal stem cells (MSCs), each exosome comprising at least one factor that is: an osteoinductive factor, a neuronal regeneration factor, an immunomodulatory factor, an extracellular matrix binding factor, or a combination thereof, wherein the at least one factor is present at a higher amount in the engineered exosome than the amount present in a naturally occurring cell-derived exosome.

2. The composition of claim 1, wherein the engineered exosomes comprise at least one osteoinductive factor, wherein the at least one osteoinductive factor is present in the engineered exosome at a higher amount than the amount present in a naturally occurring cell-derived exosome.

3. The composition of claim 2, wherein the at least one osteoinductive factor comprises let 7a, miR 218, miR 9-5p, miR 19a-3p, mir 30a-5p, miR 212-5p, and miR 323-5p.

4. The composition of claim 3, wherein the at least one osteoinductive factor comprises let 7a.

5. The composition of claim 4, wherein the amount of let 7a in the engineered exosomes is at least 10-fold higher than the amount of let 7a in the naturally occurring cell-derived exosomes.

6. The composition of claim 4, wherein the amount of let 7a in the engineered exosomes is at least 35-fold higher than the amount of let 7a in the naturally occurring cell-derived exosomes.

7. The composition of claim 3, wherein the at least one osteoinductive factor comprises miR 218.

8. The composition of claim 7, wherein the amount of miR 218 in the engineered exosomes is at least 10-fold higher than the amount of miR 218 in the naturally occurring cell-derived exosomes.

9. The composition of claim 7, wherein the amount of miR 218 in the engineered exosomes is at least 45-fold higher than the amount of miR 218 in the naturally occurring cell-derived exosomes.

10. The composition of claim 3, wherein the at least one osteoinductive factor comprises one or more of miR-9-5p, miR-19a-3p, miR-30a-5p, miR-212-5p, miR-323-5p, miR 15a, miR 15b, miR 16, miR 424, and miR 497.

11. The composition of claim 3, wherein the at least one osteoinductive factor is an miRNA that positively regulates at least one RUNX2 and/or OSX pathway member.

12. The composition of claim 10 or 11, wherein the amount of the one or more osteoinductive factors in the engineered exosomes is at least 3-fold higher than the amount of any of the one or more osteoinductive factors in the naturally-occurring cell-derived exosomes.

13. The composition of any of claims 1-12, wherein the engineered exosomes comprise at least one immunomodulatory factor, wherein the composition decreases the ratio of pro-inflammatory M1 macrophages to anti-inflammatory M2 macrophages relative to the ratio demonstrated by the activity of naturally occurring cell-derived exosome.

14. The composition of claim 13, wherein the at least one immunomodulatory factor comprises miRNAs that downregulate at least one NF [symbol] B, SOCS3, and/or IRF-5 pathway member.

15. The composition of claim 13, wherein the at least one immunomodulatory factor comprises miRNAs that upregulate at least one LXR-alpha, STATE, and/or P13/Akt pathway member.

16. The composition of claim 13, wherein the ratio of pro-inflammatory M1 macrophages to anti-inflammatory M2 macrophages is less than the ratio present in non-healing wound of bone or neuronal tissues.

17. The composition of any of claims 1-16, wherein the engineered exosomes comprise at least one neuronal regeneration factor, wherein the at least one neuronal regeneration factor is present at a higher amount than the amount present in a naturally occurring cell-derived exosome.

18. The composition of claim 17, wherein the at least one neuronal regeneration factor comprises miR 424.

19. The composition of claim 17, wherein the amount of miR 424 in the engineered exosomes is at least 10-fold higher than the amount of miR 424 in the naturally occurring cell-derived exosome.

20. The composition of claim 17, wherein the amount of miR 424 in the engineered exosome is at least 100-fold higher than the amount of miR 424 in the naturally occurring cell-derived exosomes.

21. The composition of any of claims 1-20, wherein the engineered exosomes comprise at least one extracellular matrix binding factor, wherein the at least one extracellular matrix binding factor is present in the engineered exosome at a higher amount than the amount present in a naturally occurring cell-derived exosome.

22. The composition of claim 21, wherein the at least one extracellular matrix binding factor comprises integrin α5.

23. The composition of claim 22, wherein the amount of integrin α5 in the engineered exosome is at least 1.5-fold higher than the amount of integrin α5 present in a naturally occurring cell-derived exosome.

24. The composition of any of claims 21-23, wherein the at least one extracellular matrix binding factor increases the binding affinity or rate to one or more components of the extracellular matrix and/or extracellular matrix-derivative peptides in a dose-dependent manner.

25. The composition of claim 24, wherein the components of the extracellular matrix comprise one or more of proteins, glycoproteins, proteoglycans, and polysaccharides.

26. The composition of claim 25, wherein the one or more components of extracellular matrix comprises one or more of COL1 and FN1.

27. The composition of claim 1, wherein the engineered exosomes comprise an osteoinductive factor and integrin α5 present at a higher amount than the amount present in a naturally occurring cell-derived exosome.

28. The composition of claim 1, wherein the at least one factors comprises one or more of let 7a, miR 218, miR 9-5p, miR 19a-3p, mir 30a-5p, miR 212-5p, miR 323-5p, miR 15a, miR 15b, miR 16, miR 424, miR 497, miR 424-, or integrin α5.

29. The composition of claim 1, wherein the at least one factor comprises one or more microRNAs listed in FIG. 60.

30. The composition of any of claims 1-29, wherein the amount of the at least one factor in the exosomes is at least about 1.5-fold higher, about 3-fold higher, about 10-fold higher, about 11-fold higher, about 20-fold higher, about 50-fold higher, about 100-fold higher, about 115-fold higher, or about 200-fold higher than the amount present in the naturally occurring cell-derived exosome.

31. The composition of any one of claims 1-30, further comprising a polymer carrier.

32. The composition of claim 31, wherein the carrier comprises biocompatible polymers or oligomers that are one or more of: alginate, agarose, hyaluronic acid/hyaluronan, polyethylene glycol, poly(lactic acid), poly(vinyl alcohol), polyanhydrides, poly(glycolic acid), collagen, gelatin, heparin, glycosaminoglycans, saccharides, and self-assembling peptides.

33. The composition of claim 31 or 32, wherein the carrier is a hydrogel comprising a plurality of biocompatible polymers or oligomers cross-linked with a hydrolyzable linker.

34. The composition of claim 33, wherein the linker comprises an acrylate or a methacrylate, and optionally an ester, amide, or a combination thereof.

35. The composition of any of claims 33-34, wherein one or more of the biocompatible polymers or oligomers comprises a cell surface-binding factor.

36. The composition of claim 35, wherein the cell surface-binding factor is a component of extracellular matrix.

37. The composition of claim 35 or 36, wherein the cell surface binding factor comprises a fibronectin-derived peptide, a type I collagen-derived peptide, a peptide containing an MMP and/or enzymatic cleavage domain, or a combination thereof.

38. The composition of claim 37, wherein the fibronectin-derived peptide is RGD.

39. The composition of claim 37 or 38, wherein the collagen-derived peptide is DGEA or GFPGER.

40. The composition of any of claims 31-39, wherein the exosomes are bound to the carrier.

41. The composition of any of claims 35-39, wherein the exosomes are bound to the cell surface binding factor on the carrier.

42. The composition of any of claims 31-41, wherein the amount of the carrier is 1-15% by weight and the exosome number ranges from 1×10⁶ to 1×10¹².

43. A method of preparing a composition of any one of claims 1-42, comprising:
engineering stem cells to contain at least one factor that is: an osteoinductive factor, a neuronal regeneration factor, an immunomodulatory factor, and an extracellular matrix binding factor at a higher amount than stem cells that are not engineered; and isolating the exosome from the cells.

44. The method of claim 43, wherein engineering comprises genetic modification of the stem cells and/or and exposure of stem cells to a stimulus.

45. The method of claim 44, wherein the genetic modification of the stem cells comprises overexpression of BMP2 and/or RUNX2.

46. The method of claim 44, wherein the genetic modification of the stem cells comprises overexpression of one or more of the following factors: let 7a, miR 218, miR 9-5p, miR 19a-3p, mir 30a-5p, miR 212-5p, miR 323-5p, miR 15a, miR 15b, miR 16, miR 424, miR 497, miR 424, and integrin α5.

47. The method of claim 44, wherein the genetic modification of the stem cells comprises overexpression of at least one of BMP2, RUNX2, OSX, LXRalpha, STAT6 and/or P13/Akt pathway members.

48. The method of claim 44, wherein the genetic modification of the stem cells comprises overexpression in an exosome-specific manner.

49. The method of claim 44, wherein the exposure of stem cells to stimuli comprises culturing cells in the presence of one or more of ascorbic acid, β-glycerophosphate, and dexamethasone.

50. The method of claim 44, wherein the exposure of stem cells to stimuli comprises treating cells with TNFα.

51. The method of claim 44, wherein the exposure of stem cells to stimuli comprises exposing the stem cells to hypoxic conditions.

52. The method of any of claims 43-51, wherein the stem cells are mesenchymal stem cells.

53. The method of any of claims 43-51, wherein the stem cells are dental pulp stem cells.

54. The method of any of claims 43-53, further comprising lyophilizing the isolated exosome to obtain a lyophilized isolated exosome.

55. A method for treating a disease or disorder in an individual, comprising administering a therapeutically effective amount of the composition of any of claims 1-42 to the individual in need thereof.

56. The method of claim 55, wherein the disease or disorder is a bone disorder.

57. The method of claim 56, wherein the disease or disorder is bone defect, fracture, or a dentoalveolar disorder.

58. The method of claim 55, wherein the disease or disorder is a neurological disorder.

59. The method of claim 58, wherein the disease or disorder is ischemia, loss of neuronal function, neuronal cell death, or severed nerves.

60. The method of any of claims 55-59, wherein the composition is administered by injection.

61. The method of any of claims 55-59, wherein the composition is administered by implantation.

62. The method of any of claims 55-59, wherein the composition is administered by 3D-printed material.

63. The method of any of claims 55-62, wherein the dosage is 1×10⁶ to 1×10¹² exosomes per unit mm³ of graft, tissue, patch or injection volume or ointment.

64. A method for treating an eye disorder in an individual comprising delivering a composition of isolated exosomes to vitreous humor of the individual,
wherein the exosomes are enriched in regenerative factors endogenous to stem cells.
